SEQUENCE LISTING (21 sequences)

SEQ ID NO: 1 - protein, 147 aa, Homo sapiens
SDSEEEMKALEADFLTNMHTSKISKAHVPSWKMTLLNVCSLVNNLNSPAEETGEVHEEELVARRKLPTALDGFSLEAMLTIYQLHKICHSRAFQHWELIQEDILDTGNDKNGKEEVIKRKIPYILKRQLYENKPRRPYILKRDSYYY

SEQ ID NO: 2 - protein, 125 aa, Homo sapiens
SDSEEEMKALEADFLTNMHTSKISKAHVPSWKMTLLNVCSLVNNLNSPAEETGEVHEEELVARRKLPTALDGFSLEAMLTIYQLHKICHSRAFQHWELIQEDILDTGNDKNGKEEVIKRKIPYIL

SEQ ID NO: 3 - protein, 6 aa, Homo sapiens
KIPYIL

SEQ ID NO: 4 - protein, 13 aa, Homo sapiens
QLYENKPRRPYIL

SEQ ID NO: 5 - protein, 117 aa, Homo sapiens
SDSEEEMKALEADFLTNMHTSKISKAHVPSWKMTLLNVCSLVNNLNSPAEETGEVHEEELVARRKLPTALDGFSLEAMLTIYQLHKICHSRAFQHWELIQEDILDTGNDKNGKEEVI

SEQ ID NO: 6 - protein, 132 aa, Homo sapiens
SDSEEEMKALEADFLTNMHTSKISKAHVPSWKMTLLNVCSLVNNLNSPAEETGEVHEEELVARRKLPTALDGFSLEAMLTIYQLHKICHSRAFQHWELIQEDILDTGNDKNGKEEVIKRKIPYILKRQLYEN

SEQ ID NO: 7 - protein, 21 aa, Homo sapiens
KIPYILKRQLYENKPRRPYIL

SEQ ID NO: 8 - protein, 28 aa, Homo sapiens
KIPYILKRQLYENKPRRPYILKRDSYYY

SEQ ID NO: 9 - protein, 20 aa, Homo sapiens
QLYENKPRRPYILKRDSYYY

SEQ ID NO: 10 - protein, 243 aa, Homo sapiens
ECSQDCATCSYRLVRPADINFLACVMECEGKLPSLKIWETCKELLQLSKPELPQDGTSTLRENSKPEESHLLAKRYGGFMKRYGGFMKKMDELYPMEPEEEANGSEILAKRYGGFMKKDAEEDDSLANSSDLLKELLETGDNRERSHHQDGSDNEEEVSKRYGGFMRGLKRSPQLEDEAKELQKRYGGFMRRVGRPEWWMDYQKRYGGFLKRFAEALPSDEEGESYSKEVPEMEKRYGGFMRF

SEQ ID NO: 11 - protein, 60 aa, Homo sapiens
ECSQDCATCSYRLVRPADINFLACVMECEGKLPSLKIWETCKELLQLSKPELPQDGTSTL

SEQ ID NO: 12 - protein, 5 aa, Homo sapiens
YGGFM

SEQ ID NO: 13 - protein, 5 aa, Homo sapiens
YGGFL

SEQ ID NO: 14 - protein, 20 aa, Homo sapiens
MDELYPMEPEEEANGSEILA

SEQ ID NO: 15 - protein, 41 aa, Homo sapiens
DAEEDDSLANSSDLLKELLETGDNRERSHHQDGSDNEEEVS

SEQ ID NO: 16 - protein, 8 aa, Homo sapiens
YGGFMRGL

SEQ ID NO: 17 - protein, 12 aa, Homo sapiens
SPQLEDEAKELQ

SEQ ID NO: 18 - protein, 11 aa, Homo sapiens
VGRPEWWMDYQ

SEQ ID NO: 19 - protein, 22 aa, Homo sapiens
FAEALPSDEEGESYSKEVPEME

SEQ ID NO: 20 - protein, 29 aa, Homo sapiens
FAEALPSDEEGESYSKEVPEMEKRYGGFM

SEQ ID NO: 21 - protein, 7 aa, Homo sapiens
YGGFMRF

(19) United States
(12) Patent Application Publication
(10) Pub. No.: US 2016/0324881 A1
(43) Pub. Date: Nov. 10, 2016

(54) Neurokinin-1 Receptor Antagonists For Use In A Method Of Prevention Of Cancer

(71) Applicant: ONCOPREVENT GMBH, Hennigsdorf (DE)

(72) Inventor: Andreas BERGMANN, Berlin (DE)

(73) Assignee: ONCOPREVENT GMBH, Hennigsdorf (DE)

(21) Appl. No.: 15/108,904

(22) PCT Filed: Dec. 29, 2014
(86) PCT No.: PCT/EP2014/079365

§ 371 Date: Jun. 29, 2016

(30) Foreign Application Priority Data
Dec. 30, 2013 (EP) 13199852.8

(51) Int. Cl. A61K 31/675; A61K 9/00; A61K 45/06; A61K 31/5377

(52) U.S. Cl. CPC A61K 31/675; A61K 31/5377; A61K 9/0053; A61K 9/0019; A61K 45/06

(57) ABSTRACT

Subject matter of the present invention is a neurokinin-1 receptor antagonist for use in a method of prevention of cancer.

Particularly, subject matter of the present invention is a neurokinin-1 receptor antagonist according to the compounds of Formula (I) or a tautomer thereof or a pharmaceutically acceptable salt thereof or a pharmaceutically acceptable salt of a tautomer thereof for use in a method of preventing cancer. In a very specific embodiment the non-peptide antagonist fosaprepitant sir aprepitant is said neurokinin-1 receptor antagonist. In another specific embodiment, subject matter of the present invention is a neurokinin-1 receptor antagonist for use in a method of preventing cancer as monotherapeutic agent.

### FIELD OF THE INVENTION

The field of the invention is cancer prevention via neurokinin-1 receptor antagonists. Subject matter of the present invention is a neurokinin-1 receptor antagonist for use in a method of prevention of cancer as a cancer preventing agent. In one embodiment said neurokinin-1 receptor antagonist is selected from the compounds of Formula (I) or a tautomer thereof or a pharmaceutically acceptable salt thereof or a pharmaceutically acceptable salt of a tautomer thereof. In a specific embodiment of the invention fosaprepitant is said neurokinin-1 receptor antagonist. In another specific embodiment the herein disclosed neurokinin-1 receptor antagonists for use in a method of prevention of cancer are intended for mono application.

### BACKGROUND

Neurokinin-1 (hereinafter abbreviated NK-1) receptor antagonists represent an own class of pharmaceutical medications that are known for its antidepressant, anxiolytic, and antiemetic properties. Typically, said NK-1 receptor antagonists are given under second line treatment to address vomiting (emesis) that is commonly associated with conventional cancer chemotherapy.

A prominent example of a NK-1 receptor antagonist is the drug aprepitant. Emesis appears to consist of acute and delayed phases. So far, the discovery and development of the NK-1 receptor antagonist aprepitant have elicited its antiemetic effect in both, acute and particularly in the delayed phases of emesis.

It is further known that after binding of a ligand to the NK-1 receptor, substance P (hereinafter abbreviated SP) induces tumor cell proliferation, the migration of tumor cells (invasion and metastasis) and angiogenesis. By contrast, NK-1 receptor antagonists inhibit tumor cell proliferation as tumor cells die by apoptosis, they further block the migratory activity of tumor cells and exert antiangiogenic properties [Munoz M, Martinez-Armesto J, Covenas R: NK-1 receptor antagonists as antitumor drugs: a survey of the literature from 2000 to 2011. Expert opinion on therapeutic patents 2012, 22(7): 735-746]Chemically diverse NK-1 receptor antagonists have been identified so far. In the host they display further beneficial effects, namely anxiolytic, neuroprotective, nephroprotective, hepatoprotective, antiinflammatory and analgesic effects besides the antiemetic activity. Further, these antagonists are broad-spectrum antitumor drugs. The antitumor activities of these compounds are independent of their chemical structures and its activity is associated with their affinity for the NK-1 receptor and with the dose of the antagonist administered [Munoz M, Covenas R: NK-1 receptor antagonists: a new generation of anticancer drugs. Mini reviews in medicinal chemistry 2012, 12(7):593-599].

Accordingly, the NK-1 receptor can be considered as a target in cancer treatment and NK-1 receptor antagonists could be considered as novel antitumor drugs. As of yet, the NK-1 receptor antagonist aprepitant is used in clinical practice to address the chemotherapeutic side effects of vomiting. Aprepitant is known to exert an antitumor activity against tumor cells in vitro. Such antitumor activity, however, has not been tested in vivo, so far.

Surprisingly, according to the present invention it was shown that NK-1 receptor antagonists have a preventive anti-cancer effect.

In more detail:

Aprepitant (Emend (US, EU)) is an antiemetic chemical compound, having a molecular weight of 534.427 g/mol (http://www.drugbank.ca/drugs/DB00673). It mediates its effect by blocking the NK-1 receptor. Aprepitant is virtually water-insoluble, having a predicted water solubility of 1.94⁻² g/l (http://www.drugbank.ca/drugs/DB00673). Specifically, aprepitant is blocking signals given off by NK-1 receptors. This, therefore, decreases the likelihood of vomiting in patients. Aprepitant is usually taken as a preventative for chemotherapy-induced nausea and vomiting (CINV), which is a serious side-effect experienced by over 80% of patients who undergo chemotherapy. Aprepitant is taken orally in the form of capsules. Its average oral bioavailability is found to be around 60-65%.

One of the fundamental features of aprepitant, and a major advantage it has over other chemotherapy-induced side-effect treatments is that—while it successfully antagonizes the NK1 receptors—it has very little affinity over other receptors such as serotonin, dopamine, and corticosteroid. It is estimated that aprepitant is at least 3000 times more selective of NK1 receptors compared to these other enzyme transporter, ion channels.

Fosaprepitant (Emend for Injection (US), Ivemend (EU)) is the intravenously injectable prodrug of aprepitant and also an antiemetic drug, but having a molecular weight of 614.406 g/mol (http://www.drugbank.ca/drugs/DB06717). Fosaprepitant is water-soluble having a predicted water solubility of 6.32⁻³ g/1 (http://www.drugbank.ca/drugs/DB06717) [Munoz M, Covenas R: NK-1 receptor antagonists: a new generation of anticancer drugs. Mini reviews in medicinal chemistry 2012, 12(7):593-599].

Other known and clinically tested NK-1 receptor antagonists include casopitant, maropitant, vestipitant, L-733,060, and netupitant.

Antagonizing substance P by aprepitant as active NK-1 receptor antagonist has been recently shown to reduce brain tumor growth and also to cause cell death in the tumor cells. Dr. Harford-Wright of the University of Adelaide conducted the respective studies and demonstrated that growth of brain tumors can be halted by administration of aprepitant, noting that it is blocking substance P from binding to the NK-1 receptor, which resulted in a reduction in brain tumor growth—and it also caused cell death in the tumor cells.

### SUMMARY OF THE INVENTION

Subject matter of the present invention are NK-1 receptor antagonists for use in methods of prevention of cancer as a cancer preventing agent.

In one embodiment subject matter of the present invention are NK-1 receptor antagonists according to the compounds of Formula (I) or a tautomer thereof or a pharmaceutically acceptable salt thereof or a pharmaceutically acceptable salt of a tautomer thereof for use in methods of prevention of cancer as an cancer preventing agent.

In another embodiment said NK-1 receptor antagonist is selected from the group comprising compounds of formula I (including fosaprepitant and aprepitant), and further, casopitant, maropitant, vestipitant, L-733,060, and netupitant.

As use as a cancer preventing agent means that said compounds are administrated in order to prevent tumor formation (tumorigenesis). According to the present invention said compounds are administered as cancer preventing drugs.

It is another object of the invention that the herein disclose NK-1 receptor antagonists may be administered as a mono-agent in a method of preventing cancer, meaning not in combination with any other anticancer drugs. Even more specifically, the NK-1 receptor antagonist in accordance with the invention is a compound according to formula I, more specifically fosaprepitant or aprepitant.

### DESCRIPTION OF THE INVENTION

In one aspect subject of the invention are NK-1 receptor antagonists for use in methods of prevention of cancer as cancer preventing agent.

In a major aspect of the invention the compounds of Formula (I) or a tautomer thereof or a pharmaceutically acceptable salt thereof or a pharmaceutically acceptable salt of a tautomer thereof are intended for use in methods of prevention of cancer as cancer preventing agent. In a specific embodiment of the invention fosaprepitant or aprepitant is said neurokinin-1 receptor antagonist for use in a method of prevention of cancer as cancer preventing agent. In another specific embodiment the NK-1 receptor antagonists as disclosed herein are intended for use in methods of preventing cancer as monotherapeutics or in combination with other anticancer agents.

In a major aspect the subject matter of the invention is a compound of the general Formula (I) for use in a method of preventing cancer as a cancer preventing agent:

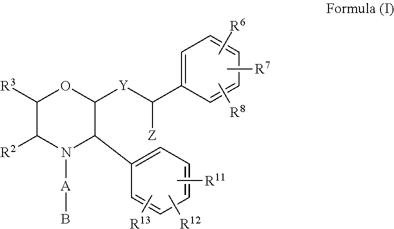

or a tautomer thereof,
or a pharmaceutically acceptable salt thereof or a pharmaceutically acceptable salt of a tautomer thereof, for use in a method of prevention of cancer, wherein

R² and R³ are independently selected from the group consisting of:
(1) hydrogen,
(2) C₁₋₆ alkyl, unsubstituted or substituted with one or more of the substituents selected from:
   (a) hydroxy,
   (b) oxo,
   (c) C₁₋₆ alkoxy,
   (d) phenyl-C₁₋₃ alkoxy,
   (e) phenyl,
   (f) —CN,
   (g) halo,
   (h) —NR⁹R¹⁰, wherein R⁹ and R¹⁰ are independently selected from:
      i. hydrogen,
      ii. C₁₋₆ alkyl,
      iii. hydroxy-C₁₋₆ alkyl, and
      iv. phenyl,
      (i) —NR⁹COR¹⁰, wherein R⁹ and R¹⁰ are as defined above,
   (j) —NR⁹CO₂R¹⁰, wherein R⁹ and R¹⁰ are as defined above,
   (k) —CONR⁹R¹⁰, wherein R⁹ and R¹⁰ are as defined above,
   (l) —COR⁹, wherein R⁹ is as defined above, and
   (m) —CO₂R⁹, wherein R⁹ is as defined above;
(3) C₂₋₆ alkenyl, unsubstituted or substituted with one or more of the substituent(s) selected from:
   (a) hydroxy,
   (b) oxo,
   (c) C₁₋₆ alkoxy,
   (d) phenyl-C₁₋₃ alkoxy,
   (e) phenyl,
   (f) —CN,
   (g) halo,
   (h) —CONR⁹R¹⁰ wherein R⁹ and R¹⁰ are as defined above,
   (i) —COR⁹ wherein R⁹ is as defined above,
   (j) —CO₂R⁹, wherein R⁹ is as defined above;
(4) C₂₋₆ alkynyl;
(5) phenyl, unsubstituted or substituted with one or more of the substituent(s) selected from:
   (a) hydroxy,
   (b) C₁₋₆ alkoxy,
   (c) C₁₋₆ alkyl,
   (d) C₂₋₅ alkenyl,
   (e) halo,
   (f) —CN,
   (g) —NO₂,
   (h) —CF₃,
   (i) —(CH₂)m-NR⁹R¹⁰, wherein m, R⁹ and R¹⁰ are as defined above,
   (j) —NR⁹COR¹⁰, wherein R⁹ and R¹⁰ are as defined above,
   (k) —NR⁹CO₂R¹⁰, wherein R⁹ and R¹⁰ are as defined above,
   (l) —CONR⁹R¹⁰, wherein R⁹ and R¹⁰ are as defined above,
   (m) —CO₂NR⁹R¹⁰, wherein R⁹ and R¹⁰ are as defined above,
   (n) —COR⁹, wherein R⁹ is as defined above;
   (o) —CO₂R⁹, wherein R⁹ is as defined above;
      and, alternatively, the groups R² and R³ are joined together to form a carbocyclic ring selected from the group consisting of:
(a) cyclopentyl,
(b) cyclohexyl,
(c) phenyl,
and wherein the carbocyclic ring is unsubstituted or substituted with one or more substituents selected from:
   (i) C₁₋₆ alkyl,
   (ii) C₁₋₆ alkoxy,
   (iii) —NR⁹R¹⁰, wherein R⁹ and R¹⁰ are as defined above,
   (iv) halo, and
   (v) trifluoromethyl;
      and, alternatively, the groups R² and R³ are joined together to form a heterocyclic ring selected from the group consisting of
(a) pyrrolidinyl,
(b) piperidinyl,
(c) pyrrolyl,
(d) pyridinyl,
(e) imidazolyl,
(t) furanyl,
(g) oxazolyl,
(h) thienyl, and
(i) thiazolyl,
and wherein the heterocyclic ring is unsubstituted or substituted with one or more substituent(s) selected from:
   (i) C₁₋₆ alkyl,
   (ii) oxo,
   (iii) C₁₋₆ alkoxy,
   (iv) —NR⁹R¹⁰, wherein R⁹ and R¹⁰ are as defined above,
   (v) halo, and
   (vi) trifluoromethyl;

R⁶, R⁷ and R⁸ are independently selected from the group consisting of:
(1) hydrogen,
(2) C₁₋₆ alkyl, unsubstituted or substituted with one or more of the substituents selected from:
   (a) hydroxy,
   (b) oxo,
   (c) C₁₋₆ alkoxy,
   (d) phenyl-C₁₋₃ alkoxy,
   (e) phenyl,
   (f) —CN,
   (g) halo,
   (h) —NR⁹R¹⁰, wherein R⁹ and R¹⁰ are as defined above,
   (i) —NR⁹COR¹⁰, wherein R⁹ and R¹⁰ are as defined above,
   (j) —NR⁹CO₂R¹⁰, wherein R⁹ and R¹⁰ are as defined above,
   (k) —CONR⁹R¹⁰, wherein R⁹ and R¹⁰ are as defined above,
   (l) —COR⁹, wherein R⁹ us as defined above, and
   (m) —CO₂R⁹, wherein R⁹ is as defined above;
(3) C₂₋₆ alkenyl, unsubstituted or substituted with one or more of the substituent(s) selected from:
   (a) hydroxy,
   (b) oxo,
   (c) C₁₋₆ alkoxy,
   (d) phenyl-C₁₋₃ alkoxy,
   (e) phenyl,
   (f) —CN,
   (g) halo,
   (h) —CONR⁹R¹⁰,wherein R⁹ and R¹⁰ are as defined above,
   (i) —COR⁹, wherein R⁹ us as defined above,
   (j) —CO₂R⁹, wherein R⁹ us as defined above;
(4) C₂₋₆ alkynyl;
(5) phenyl, unsubstituted or substituted with one or more of the substituents selected from:
   (a) hydroxy,
   (b) C₁₋₆ alkoxy,
   (c) C₁₋₆ alkyl,
   (d) C₂₋₅ alkenyl,
   (e) halo,
   (f) —CN,
   (g) —NO₂,
   (h) —CF₃,
   (i) —(CH₂)ₘ—NR⁹R¹⁰, wherein m, R⁹ and R¹⁰ are as defined above,
   (j) —NR⁹COR¹⁰, wherein R⁹ and R¹⁰ are as defined above,
   (k) —NR⁹CO₂R¹⁰, wherein R⁹ and R¹⁰ are as defined above,
   (l) —CONR⁹R¹⁰, wherein R⁹ and R¹⁰ are as defined above,
   (m) —CO₂NR⁹R¹⁰, wherein R⁹ and R¹⁰ are as defined above,
   (n) —COR⁹, wherein R⁹ is as defined above,
   (o) CO₂R⁹, wherein R⁹ is as defined above;
(6) halo,
(7) —CN,
(8) —CF₃,
(9) —NO₂,
(10) —SR¹⁴, wherein R¹⁴ is hydrogen or C₁₋₅ alkyl,
(11) —SOR¹⁴, wherein R¹⁴ is as defined above,
(12) —SO₂R¹⁴, wherein R¹⁴ is as defined above,
(13) —NR⁹COR¹⁰, wherein R⁹ and R¹⁰ are as defined above,
(14) —CONR⁹COR¹⁰, wherein R⁹ and R¹⁰ are as defined above,
(15) —NR⁹R¹⁰, wherein R⁹ and R¹⁰ are as defined above,
(16) NR⁹CO₂R¹⁰, wherein R⁹ and R¹⁰ are as defined above,
(17) hydroxy,
(18) C₁₋₆ alkoxy,
(19) COR⁹, wherein R⁹ is as defined above,
(20) CO₂R⁹, wherein R⁹ is as defined above,
(21) 2-pyridyl,
(22) 3-pyridyl,
(23) 4-pyridyl,
(24) 5-tetrazolyl,
(25) 2-oxazolyl, and
(26) 2-thiazolyl;

R¹¹, R¹² and R¹³ are independently selected from the definitions of R⁶, R⁷ and R⁸;

A is selected from the group consisting of:
(1) C₁₋₆ alkyl, unsubstituted or substituted with one or more of the substituents selected from:
(a) hydroxy,
(b) oxo,
(c) C₁₋₆ alkoxy,
(d) phenyl-C₁₋₃ alkoxy,
(e) phenyl,
(f) —CN,
(g) halo, wherein halo is fluoro, chloro, bromo or iodo,
(h) —NR⁹R¹⁰, wherein R⁹ and R¹⁰ are as defined above,
(i) —NR⁹COR¹⁰, wherein R⁹ and R¹⁰ are as defined above,
(j) —NR⁹CO₂R¹⁰, wherein R⁹ and R¹⁰ are as defined above,
(k) —CONR⁹R¹⁰, wherein R⁹ and R¹⁰ are as defined above,
(1) —COR⁹, wherein R⁹ is as defined above, and (m) —CO₂R⁹, wherein R⁹ is as defined above;
(2) C₂₋₆ alkenyl, unsubstituted or substituted with one or more of the substituent(s) selected from:
   (a) hydroxy,
   (b) oxo,
   (c) C₁₋₆ alkoxy,
   (d) phenyl-C₁₋₃ alkoxy,
   (e) phenyl,
   (f) —CN,
   (g) halo,
   (h) —CONR⁹R¹⁰, wherein R⁹ and R¹⁰ are as defined above,
   (i) —COR⁹, wherein R⁹ is as defined above, and
   (j) CO₂R⁹, wherein R⁹ is as defined above; and
(3) C₂₋₆ alkenyl;

B is a heterocycle, wherein the heterocycle is selected from the group consisting of

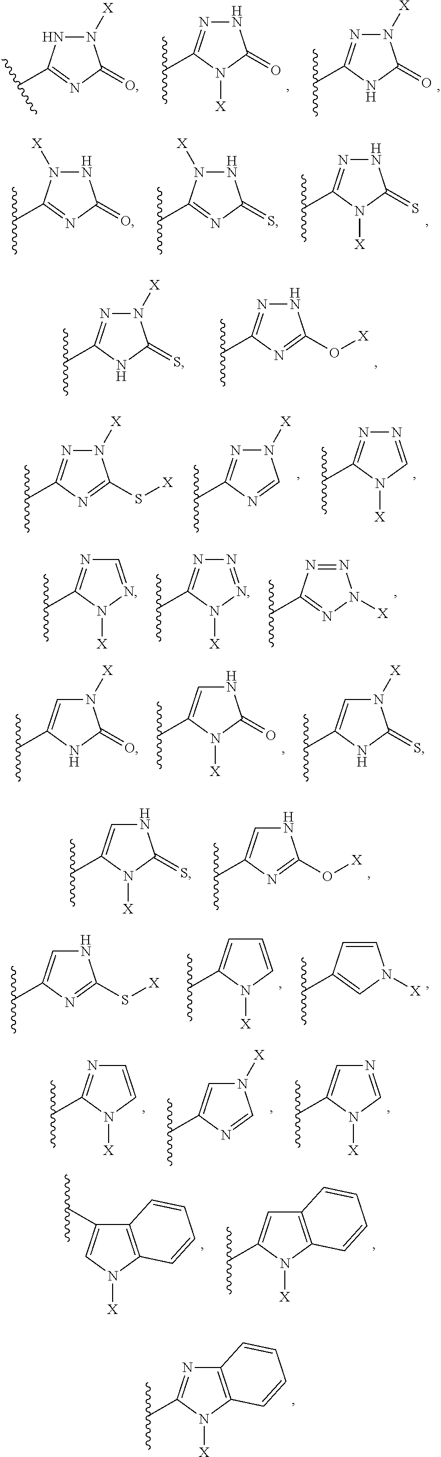

and wherein the heterocycle is substituted in addition to —X with one or more substituent(s) selected from:
(i) hydrogen;
(ii) C₁₋₆ alkyl, unsubstituted or substituted with halo, —CF₃, —OCH₃, or phenyl,
(iii) C₁₋₆ alkoxy,
(iv) oxo,
(v) hydroxy,
(vi) thioxo,
(vii) —SR⁹, wherein R⁹ is as defined above,
(viii) halo,
(ix) —CN,
(x) phenyl,
(xi) trifluoromethyl,
(xii) —(CH₂)ₘ—NR⁹R¹⁰, wherein m is 0, 1 or 2, and R⁹ and R¹⁰ are as defined above,
(xiii) —NR⁹COR¹⁰, wherein R⁹ and R¹⁰ are as defined above,
(xiv) —CONR⁹R¹⁰, wherein R⁹ and R¹⁰ are as defined above,
(xv) —CO₂R⁹, wherein R⁹ is as defined above, and
(xvi) —(CH₂)ₘ—OR⁹, wherein m and R⁹ are as defined above;

X is selected from:
(a) hydrogen,
(b) —PO(OH)₂
(c) —PO(OH)O⁻ wherein M⁺ is a pharmaceutically acceptable monovalent counterion,
(d) —PO(O⁻)₂.2M⁺,
(e) —PO(O⁻)₂.D²⁺, wherein D²⁺ is a pharmaceutically acceptable divalent counterion,
(f) —CH(R⁴)—PO(OH)O⁻.M⁺, wherein R⁴ is hydrogen or C₁₋₃ alkyl,
(g) —CH(R⁴)—PO(O⁻)₂.2M⁺, and
(h) —CH(R⁴)—PO(O⁻)₂.D²⁺;

Y is selected from the group consisting of:
(1) a single bond,
(2) —O—,
(3) —S—,
(4) —CO—,
(5) —CH₂—,
(6) —CHR¹⁵—, and
(7) —CR¹⁵R¹⁶—, wherein R¹⁵ and R¹⁶ are independently selected from the group consisting of:
   (a) C₁₋₆ alkyl, unsubstituted or substituted with one or more of the substituents selected from:
      (i) hydroxy,
      (ii) oxo,
      (iii) C₁₋₆ alkoxy,
      (iv) phenyl-C₁₋₃ alkoxy,
      (v) phenyl,
      (vi) —CN,
      (vii) halo,
      (viii) —NR⁹R¹⁰, wherein R⁹ and R¹⁰ are as defined above,
      (ix) —NR⁹COR¹⁰, wherein R⁹ and R¹⁰ are as defined above,
      (x) —NR⁹CO₂R¹⁰, wherein R⁹ and R¹⁰ are as defined above,
      (xi) —CONR⁹R¹⁰, wherein R⁹ and R¹⁰ are as defined above,
      (xii) —COR⁹, wherein R⁹ is as defined above, and
      (xiii) —CO₂R⁹, wherein R⁹ is as defined above;
   (b) phenyl, unsubstituted or substituted with one or more of the substituent(s)
      (i) hydroxy,
      (ii) C₁₋₆ alkoxy,
      (iii) C₁₋₆ alkyl,
      (iv) C₂₋₅ alkenyl,
      (v) halo,
      (vi) —CN,
      (vii) —NO₂,
      (viii) —CF₃,
      (ix) —(CH₂)ₘ—NR⁹R¹⁰, wherein m, R⁹ and R¹⁰ are as defined above,
      (x) —NR⁹COR¹⁰, wherein R⁹ and R¹⁰ are as defined above,
      (xi) —NR⁹CO₂R¹⁰, wherein R⁹ and R¹⁰ are as defined above,
      (xii) —CONR⁹R¹⁰, wherein R⁹ and R¹⁰ are as defined above,
      (xiii) —CO₂NR⁹R¹⁰, wherein R⁹ and R¹⁰ are as defined above,
      (xiv) —COR⁹, wherein R⁹ is as defined above, and
      (xv) —CO₂R⁹, wherein R⁹ is as defined above;
Z is selected from:
   (1) hydrogen,
   (2) C₁₋₆ alkyl, and
   (3) hydroxy, with the proviso that if Y is —O—, Z is other than hydroxy, or if Y is —CHR¹⁵—, then Z and R¹⁵ are optionally joined together to form a double bond.

While all of the usual routes of administration are useful with the present compounds, the preferred routes of administration are oral and intravenous. After gastrointestinal absorption or intravenous administration, the present compounds that exhibit an X which is not hydrogen or is absent are hydrolyzed or otherwise cleaved in vivo to the corresponding parent compounds of formula I, wherein X is hydrogen or X is absent, or a salt thereof. Since the parent compounds may be relatively insoluble in aqueous solutions, the instant prodrugs provide a distinct advantage by virtue of their relatively enhanced aqueous solubility. This means the so-called parent compounds are compounds of formula I, wherein X is hydrogen or X is absent, or a salt thereof. The instant prodrugs are compounds of formula I that exhibit an X as above defined which is neither H nor absent and which are hydrolyzed or otherwise cleaved in vivo to the corresponding parent compounds.

Subject matter of the present invention are NK-1 receptor antagonist for use in a method of preventing cancer as a cancer preventing agent, wherein said NK-1 receptor antagonist is selected from the group comprising a compound of Formula (I) including tautomers, pharmaceutically acceptable salts thereof or a pharmaceutically acceptable salt of said tautomers of Formula (I) and further

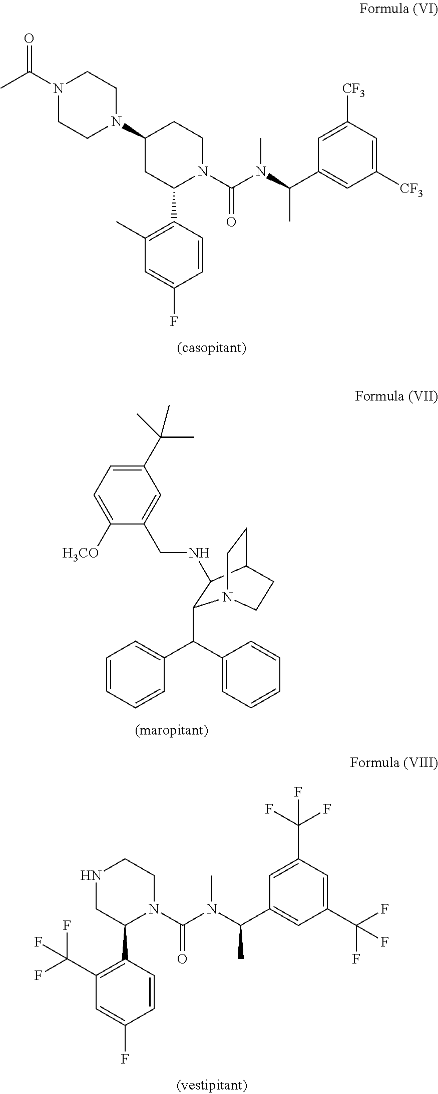 [SMILES: CC(=O)N1CCN([C@H]2CCN(C(=O)N(C)[C@H](C)c3cc(C(F)(F)F)cc(C(F)(F)F)c3)[C@H](c3ccc(F)cc3C)C2)CC1 ; COc1ccc(C(C)(C)C)cc1CNC1C2CCN(CC2)C1C(c1ccccc1)c1ccccc1 ; C[C@H](c1cc(C(F)(F)F)cc(C(F)(F)F)c1)N(C)C(=O)N1CCNC[C@@H]1c1ccc(F)cc1C(F)(F)F]

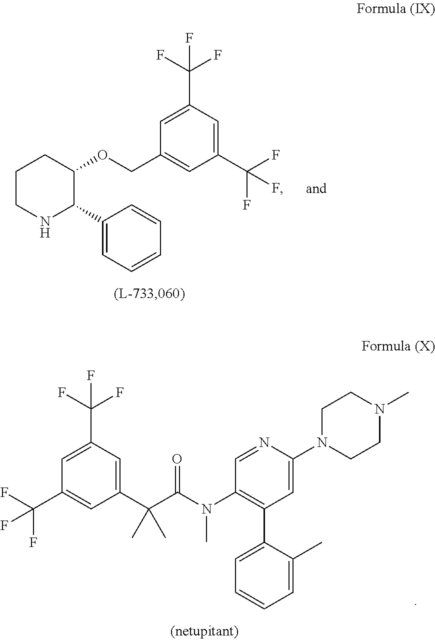 [SMILES: FC(F)(F)c1cc(CO[C@H]2CCCN[C@H]2c2ccccc2)cc(C(F)(F)F)c1 ; "and" ; Cc1ccccc1-c1cc(N2CCN(C)CC2)ncc1N(C)C(=O)C(C)(C)c1cc(C(F)(F)F)cc(C(F)(F)F)c1]

The compounds of the present invention have asymmetric centers and this invention includes all of the optical isomers and mixtures thereof.

In addition compounds with carbon-carbon double bonds may occur in Z— and E-forms with all isomeric forms of the compounds being included in the present invention.

When any variable (e.g., alkyl, aryl, R⁶, R⁷, R⁸, R⁹, R¹⁰, R¹¹, R¹², R¹³, etc.) occurs more than one time in any variable or in Formula I, its definition on each occurrence is independent of its definition at every other occurrence.

As used herein, the term “alkyl” includes those alkyl groups of a designated number of carbon atoms of either a straight, branched, or cyclic configuration. Examples of “alkyl” include methyl, ethyl, propyl, isopropyl, butyl, iso-sec- and tert-butyl, pentyl, hexyl, heptyl, 3-ethylbutyl, cyclopropyl, cyclobutyl, cyclopentyl, cyclohexyl, cycloheptyl, norbornyl, and the like. “Alkoxy” represents an alkyl group of indicated number of carbon atoms attached through an oxygen bridge, such as methoxy, ethoxy, propoxy, butoxy and pentoxy. “Alkenyl” is intended to include hydrocarbon chains of a specified number of carbon atoms of either a straight- or branched-configuration and at least one unsaturation, which may occur at any point along the chain, such as ethenyl, propenyl, butenyl, pentenyl, dimethylpentyl, and the like, and includes E and Z forms, where applicable. “Halogen” or “halo”, as used herein, means, fluoro, chloro, bromo and iodo.

The compounds of the present invention are capable of forming salts with various inorganic and organic acids and bases and such salts are also within the scope of this invention. Examples of such acid addition salts (which are negative counterions defined herein as “M-”) include acetate, adipate, benzoate, benzenesulfonate, bisulfate, butyrate, camphorate, camphorsulfonate, citrate, ethanesulfonate, fumarate, hemisulfate, 2-hydroxyethylsulfonate, heptanoate, hexanoate, hydrochloride, hydrobromide, hydroiodide, lactate, malate, maleate, methanesulfonate, 2-naphthalenesulfonate, oxalate, pamoate, persulfate, picrate, pivalate, propionate, salicylate, stearate, succinate, sulfate, tartrate, tosylate (p-toluenesulfonate), and undecanoate. Base salts (which are pharmaceutically acceptable monovalent cations defined herein as “M⁺ or K⁺” or pharmaceutically acceptable divalent cations defined herein as “D²⁺”, if appropriate) include ammonium salts, alkali metal salts such as sodium, lithium and potassium salts, alkaline earth metal salts such as aluminum, calcium and magnesium salts, salts with organic bases such as dicyclohexylamine salts, N-methyl-D-glucamine, and salts with amino acids such as arginine, lysine, ornithine, and so forth. If M⁺ is a monovalent cation, it is recognized that if the definition 2M⁺ is present, each of M⁺ may be the same or different. In addition, it is similarly recognized that if the definition 2M⁺ is present, a divalent cation D²⁺ may instead be present. Also, the basic nitrogen-containing groups may be quaternized with such agents as: lower alkyl halides, such as methyl, ethyl, propyl, and butyl chloride, bromides and iodides; dialkyl sulfates like dimethyl, diethyl, dibutyl; diamyl sulfates; long chain halides such as decyl, lauryl, myristyl and stearyl chlorides, bromides and iodides; aralkyl halides like benzyl bromide and others. The non-toxic physiologically acceptable salts are preferred, although other salts are also useful, such as in isolating or purifying the product.

The salts may be formed by conventional means, such as by reacting the free base form of the product with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

In a specific embodiment subject matter of the invention is a compound of general Formula (I) including tautomers thereof, pharmaceutically acceptable salts thereof or a pharmaceutically acceptable salt of said tautomers for use in a method of preventing cancer as a cancer preventing agent, wherein:
R² and R³ are independently selected from the group consisting of:
   (1) hydrogen,
   (2) C₁₋₆ alkyl,
   (3) C₂₋₆ alkenyl, and
   (4) phenyl;
R⁶, R⁷ and R⁸ are independently selected from the group consisting of:
   (1) hydrogen,
   (2) C₁₋₆ alkyl,
   (3) fluoro,
   (4) chloro,
   (5) bromo,
   (6) iodo, and
   (7) —CF₃;
R¹¹, R¹², R¹³ are independently selected from the group consisting of
   (1) fluoro,
   (2) chloro,
   (3) bromo, and
   (4) iodo;

A is unsubstituted C₁₋₆ alkyl;

B is selected from the group consisting of:

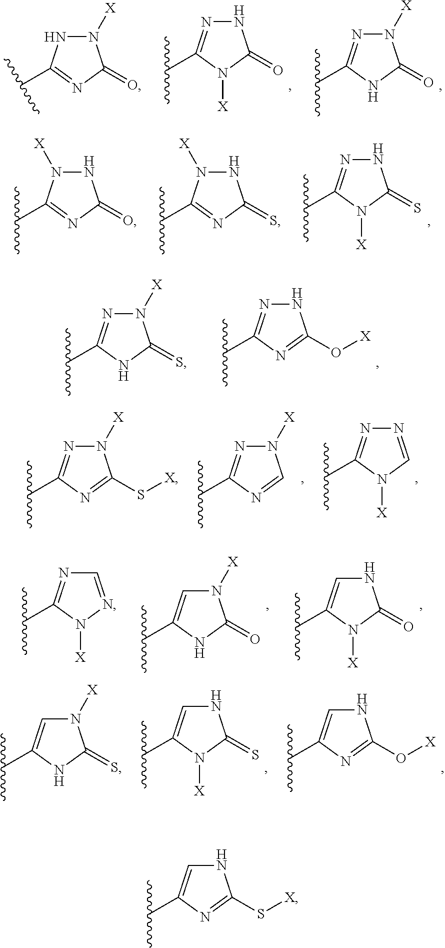

X is selected from:
   (a) hydrogen,
   (b) PO(OH)₂,
   (c) —PO(OH)O⁻.M⁺, wherein M⁺ is a pharmaceutically acceptable monovalent counterion,
   (d) —PO(O⁻)₂.2M⁺,
   (e) —PO(O⁻)₂.D²⁺, wherein D²⁺ is a pharmaceutically acceptable divalent counterion,
   (f) —CH(R⁴)—PO(OH)O⁻.M⁺, wherein R⁴ is hydrogen or methyl,
   (g) —CH(R⁴)—PO(O⁻)₂.2M⁺, wherein R⁴ is hydrogen or methyl, and
   (h) —CH(R⁴)—PO(O⁻)₂.D²⁺, wherein R⁴ is hydrogen or methyl;
Y is —O—; and
Z is hydrogen or C₁₋₄ alkyl.

In a more specific embodiment subject matter of the invention is a compound of general Formula (I) including tautomers thereof, pharmaceutically acceptable salts thereof or a pharmaceutically acceptable salt of said tautomers for use in a method of preventing cancer as a cancer preventing agent,

wherein Z is C₁₋₄ alkyl.

In an even more specific embodiment subject matter of the invention is a compound of general Formula (I) including tautomers, pharmaceutically acceptable salts thereof or a pharmaceutically acceptable salt of said tautomers for use in the prevention of cancer as a cancer preventing agent, wherein —Z is —CH₃.

In an even more specific embodiment subject matter of the invention is a compound of Formula (I) including tautomers, pharmaceutically acceptable salts thereof or a pharmaceutically acceptable salt of said tautomers for use in a method of prevention of cancer as a cancer preventing agent, wherein -A is —CH₂— or —CH(CH₃)—.

In an even more specific embodiment subject matter of the invention is a compound of Formula (I) including tautomers, pharmaceutically acceptable salts thereof or a pharmaceutically acceptable salt of said tautomers for use in a method of prevention of cancer, as a cancer preventing agent wherein —B is selected from the group consisting of

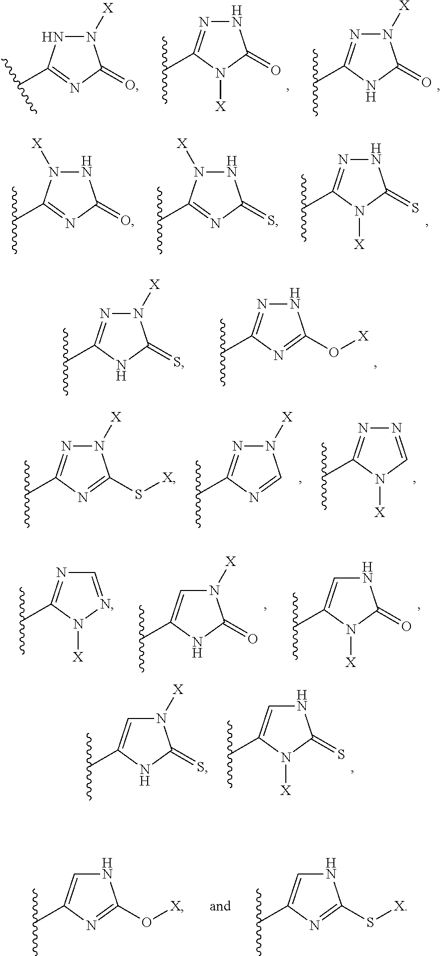

In an even more specific embodiment subject matter of the invention is a compound of general Formula (I) including tautomers, pharmaceutically acceptable salts thereof or a pharmaceutically acceptable salt of said tautomers for use in a method of prevention of cancer as a cancer preventing agent, wherein -A-B is selected from the group consisting of

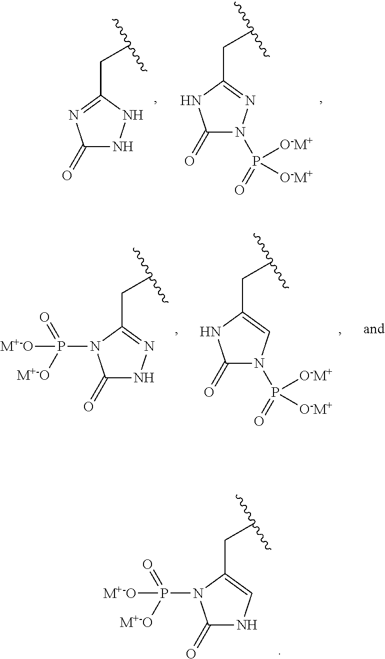

In an even more specific embodiment subject matter of the invention is a compound of Formula (I) including tautomers, pharmaceutically acceptable salts thereof or a pharmaceutically acceptable salt of said tautomers for use in the prevention of cancer as a cancer preventing agent, wherein —X is selected from the group consisting of:
(a) hydrogen,
(b) —PO(OH)₂,
(c) —PO(O)₂.2M⁺, wherein M⁺ is a pharmaceutically acceptable monovalent counterion, and
(d) —PO(O)₂.D²⁺, wherein D²⁺ is a pharmaceutically acceptable divalent counterion.

In another specific embodiment subject matter of the invention is a compound of general Formula (I) including tautomers, pharmaceutically acceptable salts thereof or a pharmaceutically acceptable salt of said tautomers for use in a method of prevention of cancer as a cancer preventing agent, having the structural Formula (II):

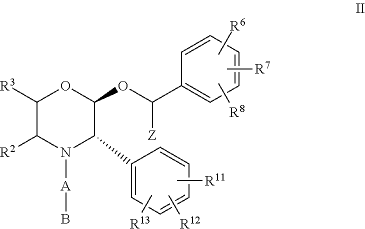

or a pharmaceutically acceptable salt thereof, wherein R², R³, R⁶, R⁷, R⁸, R¹¹, R¹², R¹³, -A, —B and —Z are as defined for general Formula (I).

In another specific embodiment subject matter of the invention is a compound according to the general Formula (I) including tautomers, pharmaceutically acceptable salts thereof or a pharmaceutically acceptable salt of said tautomers for use in a method of prevention of cancer as a cancer preventing agent, having the structural Formula (III):

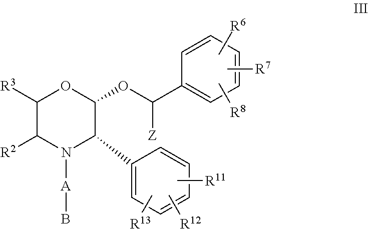

or a pharmaceutically acceptable salt thereof, wherein R², R³, R⁶, R⁷, R⁸, R¹¹, R¹², R¹³, -A, —B, and —Z are as defined in general Formula (I).

In a very specific embodiment subject matter of the invention is a compound of Formula (I) including tautomers, pharmaceutically acceptable salts thereof or a pharmaceutically acceptable salt of said tautomers for use in a method of prevention of cancer as a cancer preventing agent, which is selected from the group consisting of:
(1) 2-(R)-(1-(R)-(3,5-bis(trifluoromethyl)phenyl)ethoxy)-3-(S)-(4-fluoro)-phenyl-4-(3-(4-monophosphoryl-5-oxo-1H-1,2,4-triazolo)methyl)morpholine;
(2) 2-(R)-(1-(R)-(3,5-bis(trifluoromethyl)phenyl)ethoxy)-3-(S)-(4-fluoro)-phenyl-4-(3-(1-monophosphoryl-5-oxo-4H-1,2,4-triazolo)methyl)morpholine;
(3) 2-(R)-(1-(R)-(3,5-bis(trifluoromethyl)phenyl)ethoxy)-3-(S)-(4-fluoro)-phenyl-4-(3-(2-monophosphoryl-5-oxo-1H-1,2,4-triazolo)methyl)morpholine;
(4) 2-(R)-(1-(R)-(3,5-bis(trifluoromethyl)phenyl)ethoxy)-3-(S)-(4-fluoro)-phenyl-4-(3-(5-oxyphosphoryl-1H-1,2,4-triazolo)-methyl)morpholine;
(5) 2-(S)-(1-(R)-(3,5-bis(trifluoromethyl)phenyl)ethoxy)-3-(S)-(4-fluoro)-phenyl-4-(3-(1-monophosphoryl-5-oxo-4H-1,2,4-triazolo)methyl)morpholine;
(6) 2-(S)-(1-(R)-(3,5-bis(trifluoromethyl)phenyl)ethoxy)-3-(S)-(4-fluoro)-phenyl-4-(3-(1-monophosphoryl-5-oxo-4H-1,2,4-triazolo)methyl)morpholine
or a pharmaceutically acceptable salt thereof.

In another very specific embodiment subject matter of the invention is a compound of Formula (I) including tautomers, pharmaceutically acceptable salts thereof or a pharmaceutically acceptable salt of said tautomers for use in a method of prevention of cancer as a cancer preventing agent, which is:
2-(R)-(1-(R)-(3,5-bis(trifluoromethyl)phenyl)ethoxy)-3-(S)-(4-fluoro)-phenyl-4-(3-(1-monophosphoryl-5-oxo-4H-1,2,4-triazolo)methyl)morphonline;
or a pharmaceutically acceptable salt thereof.

In another very specific embodiment subject matter of the invention is a compound of Formula (I) including tautomers, pharmaceutically acceptable salts thereof or a pharmaceutically acceptable salt of said tautomers for use in a method of prevention of cancer as a cancer preventing agent, which is:

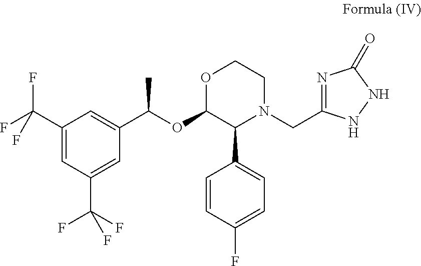 [SMILES: C[C@@H](O[C@H]1OCCN(Cc2nc(=O)[nH][nH]2)[C@H]1c1ccc(F)cc1)c1cc(C(F)(F)F)cc(C(F)(F)F)c1]

(aprepitant)
or a pharmaceutically acceptable salt thereof.

In another very specific embodiment subject matter of the invention is a compound of Formula (I) including tautomers, pharmaceutically acceptable salts thereof or a pharmaceutically acceptable salt of said tautomers for use in a method of prevention of cancer as a cancer preventing agent, wherein the pharmaceutically acceptable salt is the bis(N-methyl-D-glucamine) salt.

In another very specific embodiment subject matter of the invention is a compound of Formula (I) including tautomers, pharmaceutically acceptable salts thereof or a pharmaceutically acceptable salt of said tautomers for use in a method of prevention of cancer as a cancer preventing agent, which is
2-(R)-(1-(R)-(3,5-bis(trifluoromethyl)phenyl)ethoxy)-3-(S)-(4-fluoro)-phenyl-4-(3-(1-monophosphoryl-5-oxo-4H-1,2,4-triazolo)methyl)morpholine, bis(N-methyl-D-glucamine).

In another specific embodiment subject matter of the invention is a compound according to the general Formula (I) selected from a group of compounds as depicted below:

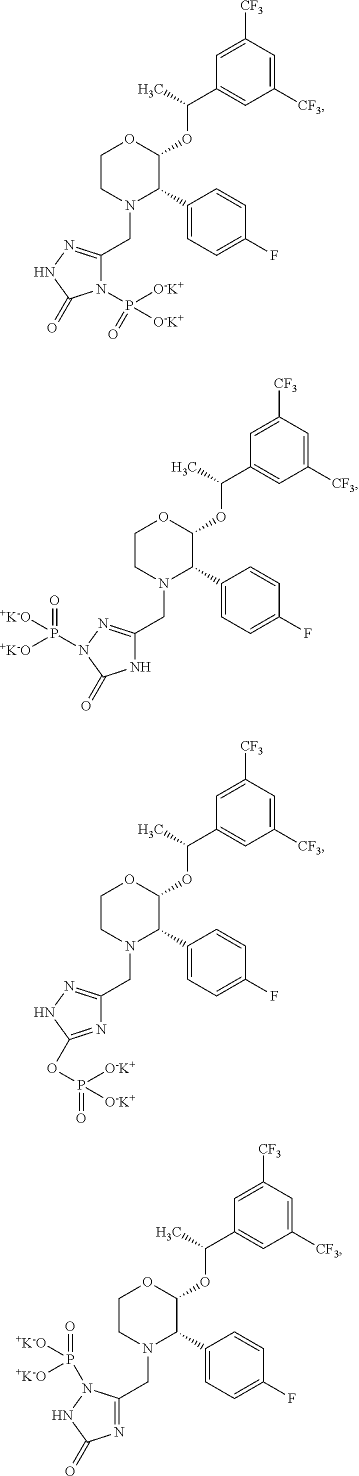

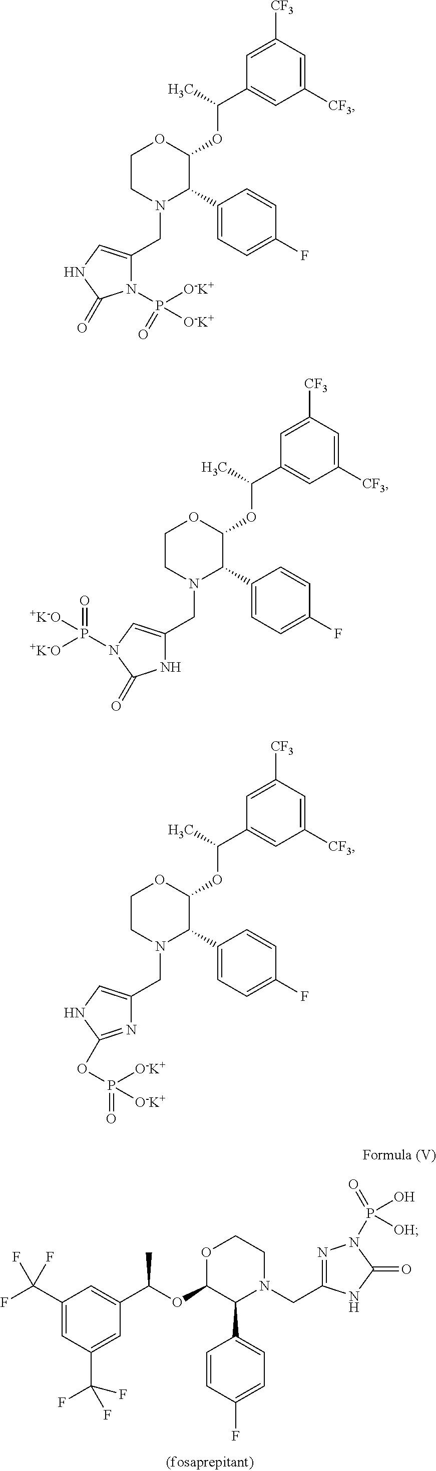

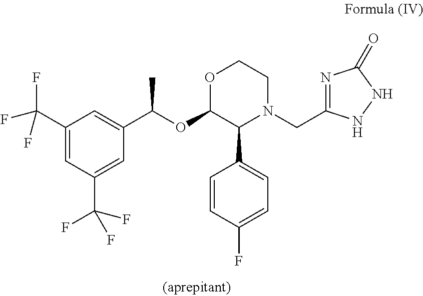

including tautomers thereof, pharmaceutically acceptable salts thereof or a pharmaceutically acceptable salt of said tautomers for use in a method of prevention of cancer,
wherein K⁺ is a pharmaceutically acceptable monovalent counterion.

In another specific embodiment subject matter of the invention is a compound selected from the compounds of the above depicted formulae for use in a method of prevention of cancer as a cancer preventing agent,
wherein K⁺ is N-methyl D-glucamine.

In another specific embodiment subject matter of the invention is a compound according to the general Formula (I) as depicted below:

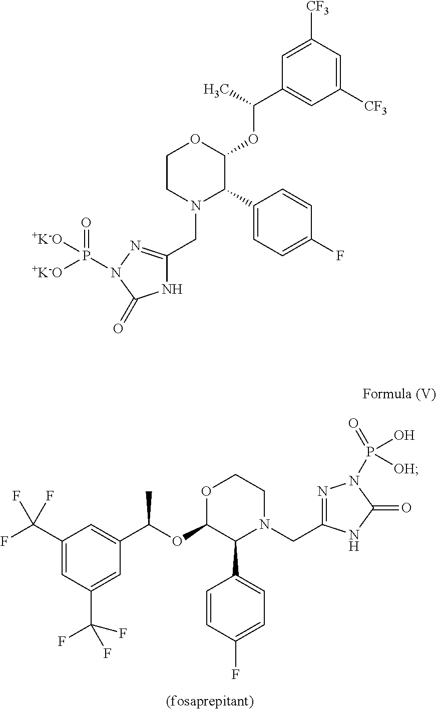

including tautomers, pharmaceutically acceptable salts thereof or a pharmaceutically acceptable salt of said tautomers for use in a method of prevention of cancer,
wherein K⁺ is a pharmaceutically acceptable monovalent counterion.

In another specific embodiment subject matter of the invention is a compound of the general Formula (I) as depicted above for use in a method of prevention of cancer as a cancer preventing agent, including tautomers, pharmaceutically acceptable salts thereof or a pharmaceutically acceptable salt of said tautomers,

wherein K⁺ is N-methyl-D-glucamine.

Compounds for use in a method of preventing cancer as a cancer preventing agent in accordance with the invention may be also selected from the group comprising the compounds of the following formulae:

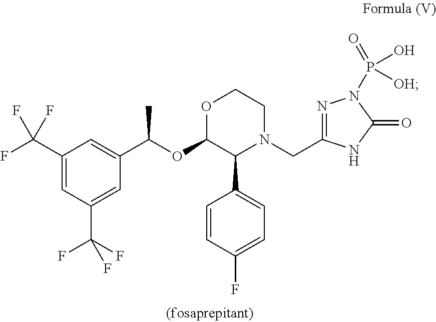 [SMILES: C[C@@H](O[C@H]1OCCN(Cc2nn(P(=O)(O)O)c(=O)[nH]2)[C@H]1c1ccc(F)cc1)c1cc(C(F)(F)F)cc(C(F)(F)F)c1]

Compounds for use in a method of preventing cancer as a cancer preventing agent in accordance with the invention may be also selected from the group comprising the compounds of the following formulae:

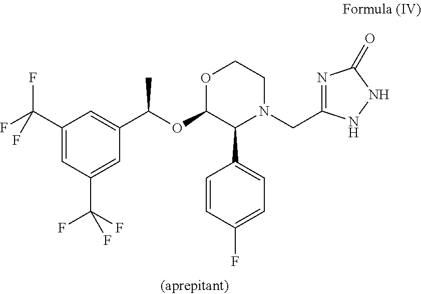 [SMILES: C[C@@H](O[C@H]1OCCN(Cc2nc(=O)[nH][nH]2)[C@H]1c1ccc(F)cc1)c1cc(C(F)(F)F)cc(C(F)(F)F)c1]

Subject matter of the present invention is a NK-1 receptor antagonist for use in a method of prevention of cancer, wherein said NK-1 receptor antagonist is selected from the group comprising Formula (I) including tautomers, pharmaceutically acceptable salts thereof or a pharmaceutically acceptable salt of said tautomers of Formula (I),

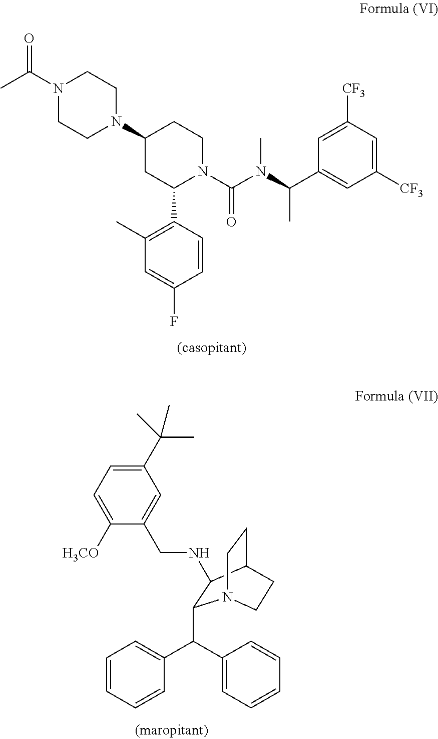 [SMILES: CC(=O)N1CCN([C@H]2CCN(C(=O)N(C)[C@H](C)c3cc(C(F)(F)F)cc(C(F)(F)F)c3)[C@H](c3ccc(F)cc3C)C2)CC1 ; COc1ccc(C(C)(C)C)cc1CNC1C2CCN(CC2)C1C(c1ccccc1)c1ccccc1]

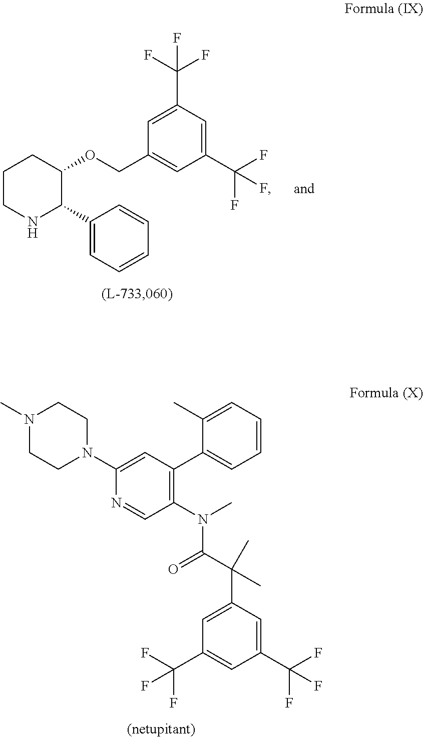 [SMILES: FC(F)(F)c1cc(CO[C@H]2CCCN[C@H]2c2ccccc2)cc(C(F)(F)F)c1 ; "and" ; Cc1ccccc1-c1cc(N2CCN(C)CC2)ncc1N(C)C(=O)C(C)(C)c1cc(C(F)(F)F)cc(C(F)(F)F)c1]

and wherein said NK-1 receptor antagonist is administered orally.

In one embodiment of the invention said NK-1 receptor antagonist is for use in a method of prevention of cancer, wherein said NK-1 receptor antagonist is aprepitant including tautomers, pharmaceutically acceptable salts thereof or a pharmaceutically acceptable salt of said tautomers and wherein said NK-1 receptor antagonist is administered orally.

In one embodiment of the invention said NK-1 receptor antagonist is for use in a method of prevention of cancer wherein said NK-1 receptor antagonist is selected from the group comprising Formula (I) including tautomers, pharmaceutically acceptable salts thereof or a pharmaceutically acceptable salt of said tautomers of Formula (I),

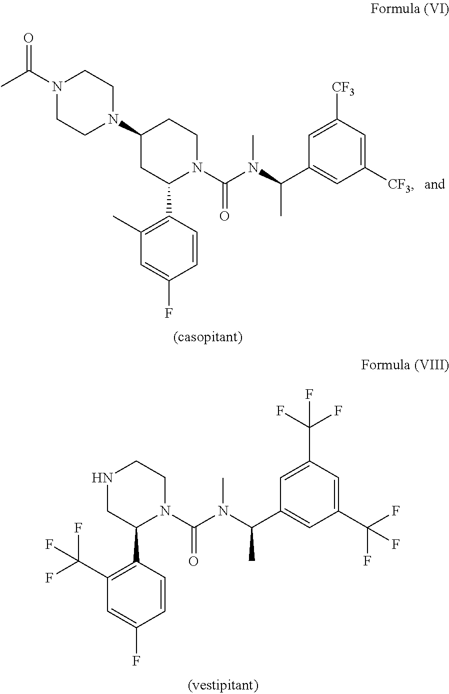 [SMILES: CC(=O)N1CCN([C@H]2CCN(C(=O)N(C)[C@H](C)c3cc(C(F)(F)F)cc(C(F)(F)F)c3)[C@H](c3ccc(F)cc3C)C2)CC1 ; C[C@H](c1cc(C(F)(F)F)cc(C(F)(F)F)c1)N(C)C(=O)N1CCNC[C@@H]1c1ccc(F)cc1C(F)(F)F]

and wherein said NK-1 receptor antagonist is administered intravenously.

In one embodiment said NK-1 receptor antagonist is for for use in a method of prevention of cancer wherein said NK-1 receptor antagonist is fosaprepitant including tautomers, pharmaceutically acceptable salts thereof or a pharmaceutically acceptable salt of said tautomers and wherein said NK-1 receptor antagonist is administered intravenously.

The person skilled in the art understands that the expression “tautomer(s) thereof” with the context of the invention denotes isomers (or rather constitutional isomers) of the compounds of either of the Formulae (I), (II), and (III) that readily interconvert by a commonly known chemical reaction referenced as “tautomerization”. As the person skilled in the art further understands, this reaction commonly results in the mere formal migration of a hydrogen atom or a proton, accompanied by a switch of a single bond and adjacent double bond. The concept of tautomerizations is also referenced as tautomerism. Because of the rapid interconversion, tautomers are generally considered to be still the same chemical compound. This fact renders it superfluous to incorporate each tautomer of the NK-1 receptor antagonists of the invention by specific structural formulae.

The expression “tautomer(s) thereof” with the context of the invention also denotes isomers as just described that are safe and effective for intravenous injection in mammals, particularly in humans and that still possess the desired biological activity, which is in accordance with the invention for inducing apoptosis in cancer cells; to inhibit proliferation of cancer cells; for decelerating tumor growth; and for inhibiting the migration of tumor cells.

With the context of the invention the expression “pharmaceutically acceptable salt(s)” denotes those salts of the NK-1 receptor antagonists of the present invention that are safe and effective for intravenous injection in mammals, particularly in humans and that still possess the desired biological activity, which is in accordance with the invention for inducing apoptosis in cancer cells; to inhibit proliferation of cancer cells; for decelerating tumor growth; and for inhibiting the migration of tumor cells. Pharmaceutically acceptable salts according to the invention include salts of acidic or basic groups present in the NK-1 receptor antagonists of the invention.

With the context of the invention the expression “pharmaceutically acceptable salt(s) of said tautomers” denotes those salts of the tautomers of the NK-1 receptor antagonists in accordance with the invention that are safe and effective for intravenous injection in mammals, particularly in humans and that still possess the desired biological activity, which is in accordance with the invention for inducing apoptosis in cancer cells; to inhibit proliferation of cancer cells; for decelerating tumor growth; and for inhibiting the migration of tumor cells. Pharmaceutically acceptable salts of the tautomers of the NK-1 receptor antagonists in accordance with the invention include salts of acidic or basic groups present in the tautomers of the NK-1 receptor antagonists of the invention.

Further, known to a person skilled in the art is a process for the preparation of a compound of Formula (I), which comprises
a) contacting a compound of Formula (I):

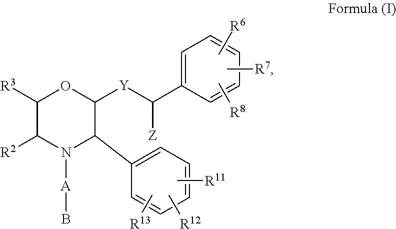

wherein A, B, Y, Z, R², R³, R⁶, R⁷, R⁸, R¹¹, R¹² and R¹³ are as defined in general Formula (I), with the exception that within such variables, X is hydrogen;
b) with an appropriate phosphoryl transfer reagent in the presence of a suitable base,
c) followed by the removal of a phosphoryl protecting group, if present, otherwise step c) is omitted,
d) so to obtain a compound of Formula (I).

Detailed methods for preparing these compounds are described e.g. in EP 0748 320 B1.

In accordance with the present invention, the above described compounds including tautomers thereof, pharmaceutically acceptable salts thereof or a pharmaceutically acceptable salt of said tautomers are intended for use in methods of prevention of cancer as a cancer preventing agent.

Said cancer may be selected from the group preferably comprising prostate, breast, colon, lung, bladder, skin, uterus, cervix, oral cavity and pharynx, stomach, ovaries cancer.

Said cancer may be also selected from the group comprising kidney, pancreas, non-Hodgekin-lymphome, leukemia, liver, esophagus, testicle, thyroid, central nervous system, larynx, gall bladder, and plasmocytome cancer as well as morbus hodgekin.

In a more specific embodiment the inventive compounds including tautomers thereof, pharmaceutically acceptable salts thereof or a pharmaceutically acceptable salt of said tautomers are useful in methods of prevention of cancer as a cancer preventing agent, wherein said cancer is selected from the group comprising: prostate, breast, colon, lung, bladder, skin, uterus, cervix, oral cavity and pharynx, stomach, ovaries, kidney, pancreas, non-Hodgekin-lymphome, leukemia, liver, esophagus, testicle, thyroid, central nervous system, larynx, gall bladder, plasmocytome, and morbus hodgekin. In one embodiment of said invention the inventive compounds including tautomers thereof, pharmaceutically acceptable salts thereof or a pharmaceutically acceptable salt of said tautomers are useful in methods of prevention of breast cancer as a breast cancer preventing agent.

In one embodiment the subject to be treated according to the present invention said subject has had cancer wherein said cancer is selected from the group comprising: prostate, breast, colon, lung, bladder, skin, uterus, cervix, oral cavity and pharynx, stomach, ovaries, kidney, pancreas, non-Hodgekin-lymphome, leukemia, liver, esophagus, testicle, thyroid, central nervous system, larynx, gall bladder, plasmocytome, and morbus hodgekin and wherein the inventive compounds including tautomers thereof, pharmaceutically acceptable salts thereof or a pharmaceutically acceptable salt of said tautomers are useful in methods of prevention of recurrence of said cancer. In one embodiment said cancer is breast cancer, in one particular embodiment said breast cancer is triple negative breast cancer. Breast cancer may be classified into 5 Subtypen subtypes that may be distinguished on the basis of the expression of the estrogene receptor (ER), the expression of the progesterone receptor (PR), the expression of the human epiderminal growth factor receptor type 2 (HER2) and expression of Ki67 (cell proliferationmarker). One subtype of breast cancer is called triple negative that means ER and/or PR absent, and HER2 negative.

The inventive compounds including tautomers thereof, pharmaceutically acceptable salts thereof or a pharmaceutically acceptable salt of said tautomers are especially suited for the prevention or treatment of triple negative breast cancer.

Thus, in one embodiment of the invention subject matter of the present invention is the inventive compounds including tautomers thereof, pharmaceutically acceptable salts thereof or a pharmaceutically acceptable salt of said tautomers for use in treatment of triple negative breast cancer. Another embodiment of the present invention is the inventive compounds including tautomers thereof, pharmaceutically acceptable salts thereof or a pharmaceutically acceptable salt of said tautomers for use in a method of prevention of triple negative breast cancer. In particular in a subject that has had triple negative breast cancer and has been cured and wherein the inventive compounds including tautomers thereof, pharmaceutically acceptable salts thereof or a pharmaceutically acceptable salt of said tautomers for use in a method of prevention of recurrence of triple negative breast cancer.

In a particular embodiment aprepitant or fosaprepitant may be used in the above-mentioned methods of prevention or treatment of cancer, in particular breast cancer, in particular triple negative breast cancer. Said inventive compounds, e.g. aprepitant or fosaprepitant may be used in combination with chemotherapeutic agents, as eg. tamoxifen.

In one particular embodiment dosing and importance of the inventive compounds are of special importance, which will be described below in greater detail.

Moreover, the inventors have found surprisingly and unexpectedly that the herein provided compounds are useful in methods of prevention of cancer as cancer preventing agents without the additional use of a chemotherapeutic agent. In the entire context of the invention “compounds for use in methods of prevention of cancer as cancer preventing agents” means that those compounds exhibit an anticancer effect without additional use of a chemotherapeutic agent.

In one embodiment said NK-1 receptor antagonist is to be used as monotherapeutic, i.e. not in combination with another anticancer or cancer preventing drug.

In one embodiment said NK-1 receptor antagonist is not combined with a chemotherapeutic agent.

In another embodiment said NK-1 receptor antagonist is combined with another anticancer or cancer preventing drug, e.g. with a chemotherapeutic agent.

The person skilled in the art will understand that the compounds of the present invention may be used in combination with other cancer preventing agents, e.g. chemotherapeutic agents. Examples of such chemotherapeutic agents include alkylating agents, for example, nitrogen mustards) ethyleneimine compounds, alkyl sulfonates and other compounds with an alkylating activity such as nitrosoureas, cisplatin, and dacarbazine; antimetabolites, for example, folic acid, purine or pyrimidine antagonists; mitotic inhibitors, for example, vinca alkaloids and derivatives of podophyllotoxin; and cytotoxic antibiotics. Particular examples of chemotherapeutic agents are described, for example, by D. J. Stewart in “Nausea and Vomiting: Recent Research and Clinical Advances”, Eds. J. Kucharczyk, et. al., CRC Press Inc., Boca Raton, Fla., USA (1991), pages 177-203, especially page 188. Examples of such chemotherapeutic agents may also include aromatase inhibitors or estradiol receptor antagonist, in particular in case of breast cancer. Raloxifene and Tamoxifen are estrogen receptor modulators that are used for treatment and prevention of cancer, in particular breast cancer. Exemestane and Anastrole are aromatase inhibitors that are used for treatment and prevention of cancer, in particular breast cancer. All these chemotherapeutic agents may be used in combination with the NK1 receptor antagonists of the present invention.

Commonly used chemotherapeutic agents include cisplatin, dacar-bazine (OTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, tamoxifen and chlorambucil [R. J. Gralla, et. al., Cancer Treatment Reports, 68(1), 163-172 (1984)].

The term “chemotherapeutic agent(s)” with the context of the instant invention also denotes the following drugs in alphabetical order:

In one embodiment of the invention the chemotherapeutic agents are selected from the group comprising:

Actinomycin D, Altretamin, Anagrelid, Asparaginase, Atrasentan, Azacitidin, Bendamustin, Bleomycin, Busulfan, Capecitabin, Carboplatin, Carmustin, Chlorambucil, Cisplatin, Cladribin, Cyclophosphamid, Cytabrabin, Dacarbazin, Dasatinib, Daunorubicin, Decitabin, Demecolcin, Docetaxel, Doxifluridin, Doxorubicin, Epirubicin, Eribulin, Erlotinib, Estramustin, Etoposid, Fludarabin, 5-Fluoruracil, Fotemustin, Gefitinib, Gemicitabin, Hydroxycarbamid, Idarubicin, Ifosfamid, Imatinib, Irinotecan, Lapatinib, Lomustin, Mechlorethamin, Mercaptopurin, Methotrexat, Mitomycin, Mitoxantron, Nilotinib, Nimustin, Oxaliplatin, Paclitaxel, Pemetrexed, Pentostatin, Procarbazin, Satraplatin, Semustin, Sorafenib, Streptozocin, Sunitinib, Tamoxifen, Tegafur, Temozolomid, Teniposid, Thiotepa, Tioguanin, Topocetan, Vinblastin, Vincristin, Vindesin,Vinorelbin and Vorinostat.

This means, in another specific embodiment of the present invention the compounds of the present invention effective as cancer preventing agents even when not used in combination with any of the afore-mentioned chemotherapeutic agents, particularly alkylating agents, for example, nitrogen mustards, ethyleneimine compounds, alkyl sulfonates and other compounds with an alkylating action such as nitrosoureas, cisplatin, and dacarbazine; antimetabolites, for example, folic acid, purine or pyrimidine antagonists; mitotic inhibitors, for example, vinca alkaloids and derivatives of podophyllotoxin; and cytotoxic antibiotics more specifically the NK-1 receptor antagonists according to the present invention are not used in combination with any of chemotherapeutic agents selected from cisplatin, dacar-bazine (OTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, and chlorambucil. Thus, in one embodiment the compounds of the present invention may be used in combination with another cancer preventing drug. In another embodiment the compounds of the present invention are not used in combination with another cancer preventing drug.

Thus, in another specific embodiment subject matter of the invention is a non-peptide NK-1 receptor antagonist according to the present invention including tautomers thereof, pharmaceutically acceptable salts thereof or a pharmaceutically acceptable salt of said tautomers for use in a method of prevention of cancer as a cancer preventing agent, wherein said treatment is not combined with a treatment of any of cancer preventing agents, e.g., the chemotherapeutic agents mentioned above. Thus, in another specific embodiment subject matter of the invention is a non-peptide NK-1 receptor antagonist according to the present invention including tautomers thereof, pharmaceutically acceptable salts thereof or a pharmaceutically acceptable salt of said tautomers for use in a method of prevention of cancer as a cancer preventing agent wherein said treatment is not combined with a treatment of any of cancer preventing agents, e.g., the chemotherapeutic agents mentioned above and wherein the patient that is treated with a non-peptide NK-1 receptor antagonist according to the present invention exhibits an intolerance or exhibits side effects because of therapy with a chemotherapeutic agent.

In a specific embodiment subject matter of the invention is a non-peptide NK-1 receptor antagonist according to the present invention including tautomers, pharmaceutically acceptable salts thereof or a pharmaceutically acceptable salt of said tautomers for use in a method of prevention of cancer as a cancer preventing agent as monotherapeutic.

“First line treatment” with the context of the present invention denotes the primary therapy and/or preventive treatment that is usually recommended for a certain indication on the basis of clinical evidence for its efficacy in the population at large. If a first line treatment either fails to resolve the issue or produces intolerable side effects, additional agents commonly referred to as second line therapies may be substituted or added to the treatment regimen of first line treatment. With the context of the present invention and the general indication of cancer the first line treatment are commonly known chemotherapeutic agents.

In this regard, as of yet compounds according to Formula (I) have been used only in the form of prodrugs; especially for the treatment of emesis. Surprisingly and unexpectedly, the inventors have found that these compounds are very useful in methods of preventing cancer itself as cancer preventing agents.

Specifically, an object of the invention is the use of the herein provided non-peptide NK-1 receptor antagonists including tautomers, pharmaceutically acceptable salts thereof or a pharmaceutically acceptable salt of said tautomers for use in methods of prevention of cancer as a cancer preventing agent for inducing apoptosis in cancer cells.

Another object of the invention is the use of said non-peptide NK-1 receptor antagonists including tautomers, pharmaceutically acceptable salts thereof or a pharmaceutically acceptable salt of said tautomers for use in the prevention of cancer as a cancer preventing agent for decelerating tumor growth.

Another object of the invention is the use of said non-peptide NK-1 receptor antagonists including tautomers, pharmaceutically acceptable salts thereof or a pharmaceutically acceptable salt of said tautomers for use in the prevention of cancer for inhibition of tumor cell proliferation and angiogenesis.

In a specific and preferred embodiment of the present invention the herein disclosed NK-1 receptor antagonists including tautomers, pharmaceutically acceptable salts thereof or a pharmaceutically acceptable salt of said tautomers for use in a method of prevention of cancer are intended to be injected intravenously. The expression “intravenous injection” or the like denotes the infusion of the herein disclosed compounds as liquid substances directly into a vein of a subject.

In another specific and preferred embodiment of the present invention the herein disclosed NK-1 receptor antagonists including tautomers, pharmaceutically acceptable salts thereof or a pharmaceutically acceptable salt of said tautomers for use in a method of prevention of cancer are intended to be administered orally.

Typically, but not limited to, the treatment window of the NK-1 receptor antagonists of the invention including tautomers, pharmaceutically acceptable salts thereof or a pharmaceutically acceptable salt of said tautomers for use in a method of prevention of cancer is 2 days till 1 month, potentially to be replicated. In another embodiment typically, but not limited to, the treatment window of the NK-1 receptor antagonists of the invention including tautomers, pharmaceutically acceptable salts thereof or a pharmaceutically acceptable salt of said tautomers for use in a method of prevention of cancer is at least 3 days, at least 10 days, at least 50 days, or up to 1 year, or up to 2 years or up to 5 years, potentially to be replicated. The daily dose may be administered twice a day.

Further subject matter of the present invention is a pharmaceutical composition for use in a method of prevention of cancer comprising at least one pharmaceutically acceptable carrier and an effective amount of a NK-1 receptor antagonist according to the general Formula (I) of the present invention.

Subject matter of the present invention is a pharmaceutical composition according to the invention for use in a method of prevention of cancer, wherein the pharmaceutically acceptable carrier comprises water.

Subject matter of the present invention is a pharmaceutical composition according to the invention for use in a method of prevention of cancer, wherein the pharmaceutically acceptable carrier comprises a physiologically acceptable saline solution.

Subject matter of the present invention is a pharmaceutical composition according to the invention for use in a method of prevention of cancer, wherein said formulation is administered as an injectable formulation.

The pharmaceutical compositions of this invention can be used in the form of a pharmaceutical preparation, for example, in solid, semisolid or liquid form, which contains one or more of the compounds of the present invention, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The carriers which can be used are water, glucose, lactose, gum acacia, gelatin, mannitol, starch paste, magnesium trisilicate, talc, corn starch, keratin, colloidal silica, potato starch, urea and other carriers suitable for use in manufacturing preparations, in solid, semisolid, or liquid form, and in addition auxiliary, stabilizing, thickening and coloring agents and perfumes may be used. The active object compound is included in the pharmaceutical composition in an amount sufficient to produce the desired effect upon the process or condition of the disease.

The liquid forms in which the novel compositions of the present invention may be incorporated for administration orally or by injection include aqueous solution, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, to methylcellulose, polyvinylpyrrolidone or gelatin.

The compounds of this invention may be administered to patients (animals and human) in need of such treatment in dosages that will provide optimal pharmaceutical efficacy. The dose will vary from patient to patient depending upon the nature and severity of disease, the patient's weight, special diets then being followed by a patient, concurrent medication, and other factors which those skilled in the art will recognize.

In one embodiment of the invention said NK-1 receptor antagonist is for use in a method of treatment or prevention of cancer, wherein the dosage level of said NK-1 receptor antagonist is in an effective amount of 2 to 200 mg/kg per day, or wherein the dosage level of said NK-1 receptor antagonist is in an effective amount of 2 to 200 mg/kg per day, or in an effective amount of 10-150 mg/kg per day, or in an effective amount of 20-100 mg/kg per day. Said daily dose may be administered in single or multiple doses. In one embodiment said NK-1 receptor antagonist is administered in said daily dosis to a patient over a period of 2 days to 1 month. In another embodiment said NK-1 receptor antagonist is administered in said daily dosis to a patient over a period of at least 2-days, preferably of at least 3 days, preferably up to at least 50 days. In one embodiment said NK-1 receptor antagonist is administered in said daily dosis to a patient over a period of up to 1 year, up to 2 years or up to 5 years.

In one embodiment of the invention said NK-1 receptor antagonist is for use in a method of prevention of cancer and wherein said NK-1 receptor antagonist is administered orally, wherein the dosage level of said NK-1 receptor antagonist is in an effective amount of 2 to 200 mg/kg per day, or wherein the dosage level of said NK-1 receptor antagonist is in an effective amount of 2 to 200 mg/kg per day, or in an effective amount of 10-150 mg/kg per day, or in an effective amount of 20-100 mg/kg per day. Said daily dose may be administered in single or multiple doses.

In one embodiment said NK-1 receptor antagonist is administered in said daily dosis orally to a patient over a period of 2 days to 1 month. In another embodiment said NK-1 receptor antagonist is administered in said daily dosis to a patient over a period of at least 2 days, preferably of at least 3 days, preferably up to at least 50 day. In one embodiment said NK-1 receptor antagonist is administered in said daily dosis to a patient over a period of up to 1 year, up to 2 years or up to 5 years.

In one embodiment of the invention said NK-1 receptor antagonist is for use in a method of prevention of cancer and wherein said NK-1 receptor antagonist is administered intravenously, wherein the dosage level of said NK-1 receptor antagonist is in an effective amount of 2 to 200 mg/kg per day, or wherein the dosage level of said NK-1 receptor antagonist is in an effective amount of 2 to 200 mg/kg per day, or in an effective amount of 10-150 mg/kg per day, or in an effective amount of 20-100 mg/kg per day. Said daily dose may be administered in single or multiple doses.[UK1][S|S2]

In one embodiment said NK-1 receptor antagonist is administered in said daily dosis intravenously to a patient over a period of 2 days to 1 month. In another embodiment said NK-1 receptor antagonist is administered intravenously in said daily dosis to a patient over a period of at least 2 days, preferably of at least 3 days, preferably up to at least 50 day. In one embodiment said NK-1 receptor antagonist is administered intravenously in said daily dosis to a patient over a period In one embodiment said NK-1 receptor antagonist is administered in said daily dosis to a patient over a period of up to 1 year, up to 2 years or up to 5 years.[UK3].

In one embodiment of the invention said NK-1 receptor antagonist is for use in a method of prevention of cancer and wherein said NK-1 receptor antagonist is administered orally, wherein the dosage level of said NK-1 receptor antagonist is in an effective amount of 60 to 200 mg/kg per day, or wherein the dosage level of said NK-1 receptor antagonist is in an effective amount of more than 60 and less than 200 mg/kg per day, or in an effective amount of 100 to 200 mg/kg per day, or in an effective amount of more than 100 and less than 200 mg/kg per day, or in an effective amount of 60 to 150 mg/kg per day or more than 60 and less than 150 mg/kg per day. Said daily dose may be administered in single or multiple doses.

In one embodiment said NK-1 receptor antagonist is administered in said daily dosis orally to a patient over a period of 2 days to 1 month.

In another embodiment of the invention said NK-1 receptor antagonist is for use in a method of prevention of cancer and wherein said NK-1 receptor antagonist is administered intravenously, wherein the dosage level of said NK-1 receptor antagonist is in an effective amount of 60 to 200 [S|S4]mg/kg per day, or wherein the dosage level of said NK-1 receptor antagonist is in an effective amount of more than 60 and less than 200 mg/kg per day, or in an effective amount of 100 to 200 mg/kg per day, or in an effective amount of more than 100 and less than 200 mg/kg per day, or in an effective amount of 60 to 150 mg/kg per day or more than 60 and less than 150 mg/kg per day. Said daily dose may be administered in single or multiple doses.

In one embodiment said NK-1 receptor antagonist is administered in said daily dosis intravenously to a patient over a period of 2 days to 1 month.

The dosage of the compounds of the present invention as cancer preventing agents is considerably higher than the dosage of the compounds of the present invention when used as antiemetic agents. Dosing/dosage as antiemetic is <3 mg/kg/d. Dosing as cancer preventing agent or as therapeutics is preferably at least two times higher. Dosing as cancer preventing agent or as therapeutics is preferably at least 5 times higher. Dosing as cancer preventing agent or as therapeutics is preferably at least 10 times higher. Dosing as cancer preventing agent or as therapeutics is preferably at least twenty times higher.

Subject matter of the invention is a pharmaceutical composition for oral administration for use in a method of preventing cancer comprising a pharmaceutically acceptable carrier and an effective amount of a NK-1 receptor antagonist of at at least 2 mg/kg/d, to be administered as a single dose per day or in several smaller doses per day, i.e. 500 mg aprepitant or more per tablet, at least more than 150 mg aprepitant per tablet. It may contain more than 125 mg aprepitant per tablet.

Subject matter of the invention is a pharmaceutical composition for oral administration for use in a method of preventing cancer comprising a pharmaceutically acceptable carrier and an effective amount of a NK-1 receptor antagonist of at at least 10 mg/kg/d, to be administered as a single dose per day or in several smaller doses per day, i.e. 500 mg aprepitant or more per tablet, at least more than 100-150 mg aprepitant per tablet. It may contain e.g. more than 125 mg aprepitant per tablet.

Subject matter of the invention is a pharmaceutical composition for oral administration for use in a method of preventing cancer comprising a pharmaceutically acceptable carrier and an effective amount of a NK-1 receptor antagonist of at at least 20 mg/kg/d, to be administered as a single dose per day or in several smaller doses per day, i.e. 500 mg aprepitant or more per tablet, at least more than 100-150 mg aprepitant per tablet. It may contain e.g. more than 125 mg aprepitant per tablet.

Subject matter of the invention is a pharmaceutical composition for oral administration for use in a method of preventing cancer comprising a pharmaceutically acceptable carrier and an effective amount of a NK-1 receptor antagonist of at at least 60 mg/kg/d, to be administered as a single dose per day or in several smaller doses per day, i.e. 500 mg aprepitant or more per tablet, at least more than 200 mg aprepitant per tablet. It may contain e.g. 1 g aprepitant per tablet.

Thus, subject matter of the present invention is a pharmaceutical composition for oral administration for use in a method of preventing cancer comprising between 200 mg to 10 g NK-1 receptor antagonist. Subject matter of the present invention is also a pharmaceutical composition for oral administration for use in a method of preventing cancer comprising between 600 mg to 10 g NK-1 receptor antagonist. Instead of one single pharmaceutical composition for oral administration that is administered once per day it is possible to have multiple compositions administered per day that contain less NK-1 receptor antagonist, respectively. This may be more convenient in view of the oral administration. It may be easier to swallow. Thus, subject matter of the present invention is also a pharmaceutical composition for oral administration for use in a method of preventing cancer comprising between 200 mg to 2 g NK-1 receptor antagonist. Subject matter of the present invention is also a pharmaceutical composition for oral administration for use in a method of preventing cancer comprising between 500 mg to 3 g NK-1 receptor antagonist. Subject matter of the present invention is also a pharmaceutical composition for oral administration for use in a method of preventing cancer comprising between 600 mg to 3 g NK-1 receptor antagonist. Subject matter of the present invention is also a pharmaceutical composition for oral administration for use in a method of preventing cancer comprising between 1 to 3 g NK-1 receptor antagonist or between 1 to 2 g.

Thus, subject matter of the present invention is a pharmaceutical composition for oral administration for use in a method of preventing cancer comprising between 100-150 mg to 10 g NK-1 receptor antagonist. Subject matter of the present invention is also a pharmaceutical composition for oral administration for use in a method of preventing cancer comprising between 100-150 mg to 10 g NK-1 receptor antagonist. Instead of one single pharmaceutical composition for oral administration that is administered once per day it is possible to have multiple compositions administered per day that contain less NK-1 receptor antagonist, respectively. This may be more convenient in view of the oral administration. It may be easier to swallow. Thus, subject matter of the present invention is also a pharmaceutical composition for oral administration for use in a method of preventing cancer comprising between 100-150 mg 10 g NK-1 receptor antagonist. Subject matter of the present invention is also a pharmaceutical composition for oral administration for use in a method of preventing cancer comprising between 100-150 mg to 10 g NK-1 receptor antagonist. Subject matter of the present invention is also a pharmaceutical composition for oral administration for use in a method of preventing cancer comprising between 100-150 mg to 10 g NK-1 receptor antagonist. Subject matter of the present invention is also a pharmaceutical composition for oral administration for use in a method of preventing cancer comprising between 100-150 mg to 10 g NK-1 receptor antagonist or between 100-150 mg o 10 g.

Some of the preferred NK-1 receptor antagonist for oral, administration are selected from the group comprising: aprepitant, casopitant, maropitant, and Netupitant In one specific embodiment said pharmaceutical composition for oral administration comprises aprepitant. The pharmaceutical composition for oral administration may be administered as pharmaceutical composition for oral administration in various forms, including: tablets to swallow, chew or dissolve in water or under the tongue, capsules and chewable capsules (with a coating that dissolves in the stomach or bowel to release the medication there) time-release or sustained-release tablets and capsules (which release the medication gradually) powders or granules, teas, drops, liquid medications or syrups.

The pharmaceutical composition for oral administration may be suspended in an aqueous application solution. For instance aprepitant may suspended in such an aqueous application solution, e.g. Ora-Plus® or Ora-Sweet®.

Further, subject matter of the present invention is a pharmaceutical composition for intravenous administration for use in a method of preventing cancer comprising between 200 mg to 10 g NK-1 receptor antagonist. Such a pharmaceutical composition for intravenous application may be either a solid preformulation that is dissolved before application or a ready to use intravenous formulation. Subject matter of the present invention is also a pharmaceutical composition for intravenous administration for use in a method of preventing cancer comprising between 600 mg to 10 g NK-1 receptor antagonist. Instead of one single pharmaceutical composition for intravenous administration that is administered once per day it is possible to have multiple compositions administered per day that contain less NK-1 receptor antagonist, respectively. This may depend on the solubility of said NK-1 receptor antagonist. Thus, subject matter of the present invention is also a pharmaceutical composition for intravenous administration for use in a method of preventing cancer comprising between 200 mg to 2 g NK-1 receptor antagonist or alternatively 600 mg to 2 g either as preformulation or as ready-to-use formulation. Subject matter of the present invention is also a pharmaceutical composition for intravenous administration for use in a method of preventing cancer comprising between 500 mg to 3 g NK-1 receptor antagonist. Subject matter of the present invention is also a pharmaceutical composition for intravenous administration for use in a method of preventing cancer comprising between 600 mg to 3 g NK-1 receptor antagonist. Subject matter of the present invention is also a pharmaceutical composition for intravenous administration for use in a method of preventing cancer comprising between 1 to 3 g NK-1 receptor antagonist or between 1 to 2 g. Some of the preferred NK-1 receptor antagonist for intravenous administration are selected from the group comprising: fosaprepitant, casopitant, and Netupitant. In one specific embodiment said pharmaceutical composition for intravenous administration comprises fosaprepitant.

Further, subject matter of the present invention is a pharmaceutical composition for intravenous administration for use in a method of preventing cancer comprising between 100-150 mg to 10 g NK-1 receptor antagonist. Such a pharmaceutical composition for intravenous application may be either a solid preformulation that is dissolved before application or a ready to use intravenous formulation. Subject matter of the present invention is also a pharmaceutical composition for intravenous administration for use in a method of preventing cancer comprising between 100-160 mg to 10 g NK-1 receptor antagonist. Instead of one single pharmaceutical composition for intravenous administration that is administered once per day it is possible to have multiple compositions administered per day that contain less NK-1 receptor antagonist, respectively. This may depend on the solubility of said NK-1 receptor antagonist. Thus, subject matter of the present invention is also a pharmaceutical composition for intravenous administration for use in a method of preventing cancer comprising between 100-160 mg to 10 g NK-1 receptor antagonist or alternatively 100-160 mg to 10 g either as preformulation or as ready-to-use formulation. Subject matter of the present invention is also a pharmaceutical composition for intravenous administration for use in a method of preventing cancer comprising between 100-160 mg to 10 g NK-1 receptor antagonist. Subject matter of the present invention is also a pharmaceutical composition for intravenous administration for use in a method of preventing cancer comprising between 100-160 mg to 10 g NK-1 receptor antagonist. Subject matter of the present invention is also a pharmaceutical composition for intravenous administration for use in a method of preventing cancer comprising between 100-160 mg to 10 g NK-1 receptor antagonist or between 100-160 mg to 10 g. Some of the preferred NK-1 receptor antagonist for intravenous administration are selected from the group comprising: fosaprepitant, casopitant, and Netupitant. In one specific embodiment said pharmaceutical composition for intravenous administration comprises fosaprepitant

Subject matter of the invention is a pharmaceutical composition for use in a method of preventing cancer, wherein the pharmaceutically acceptable carrier comprises water.

Subject matter of the invention is a pharmaceutical composition for use in a method of preventing cancer, wherein the pharmaceutically acceptable carrier comprises a physiologically acceptable saline solution.

Subject matter of the invention is a pharmaceutical composition for use in a method of preventing cancer wherein said pharmaceutical composition is to be used as monotherapeutic, i.e. not in combination with another anticancer or cancer preventing drug.

Subject matter of the invention is a pharmaceutical composition for use in a method of preventing cancer wherein said pharmaceutical composition is to be used in combination with another anticancer or cancer preventing drug.

Subject matter of the invention is a pharmaceutical composition for use in a method of preventing cancer wherein said another anticancer or cancer preventing drug is a chemotherapeutic agent. Other cancer preventing drugs and chemotherapeutic agents have been mentioned above.

Subject matter of the invention is a pharmaceutical composition for use in a method of preventing cancer, wherein said pharmaceutical composition is administered to a patient over a period of 2 days up to 1 month. Subject matter of the invention is a pharmaceutical composition for use in a method of preventing cancer, wherein said pharmaceutical composition is administered to a patient over a period of at least 2 days, or at least 3 days, or at least 10 days, or at least 30 days, or at least 50 days, or over a period of up to 1 year, up to 2 years or up to 5 years.

Subject matter of the present invention is a pharmaceutical composition according to the invention for use in a method of prevention of cancer for inducing apoptosis.

Subject matter of the present invention is a pharmaceutical composition according to the invention for use in a method of prevention of cancer, in particular for inhibition of tumor cell proliferation and angiogenesis.

Subject matter of the present invention is a pharmaceutical composition according to the invention for use in a method of prevention of cancer for inhibiting migration of tumor cells.

Subject matter of the present invention is a pharmaceutical composition according to the invention for use in a method of prevention of cancer wherein said pharmaceutical composition is administered to a patient over a period of 1 month, specifically of 2 weeks, 1 week, more specifically of 2 days. Subject matter of the present invention is a pharmaceutical composition according to the invention for use in a method of prevention of cancer wherein said pharmaceutical composition is administered to a patient over a period of at least 2 days, or at least 3 days, or at least 10 days, or at least 30 days, or at least 50 days, or over a period of up to 1 year, up to 2 years or up to 5 years.

In one specific embodiment subject of to the present invention is a NK1 receptor antagonist for use in a method of prevention of cancer wherein said NK1 receptor antagonist is administered to a subject having an enhanced risk of getting cancer.

A subject having an enhanced risk of cancer may be a subject that has been cured from cancer. Thus, a subject of the present invention is a NK1 receptor antagonist for use in a method of prevention of cancer wherein said NK1 receptor antagonist is administered to a subject that has been cured from cancer. Thus, a subject of the present invention is a NK1 receptor antagonist for use in a method of prevention of cancer wherein said NK1 receptor antagonist is administered to a subject in order to prevent re-occurrence of cancer.

A subject having an enhanced risk of getting cancer may be a female that has been cured from breast cancer and wherein the NK1 receptor antagonist is administered to a subject in order to prevent recurrence of cancer. In one embodiment said female has had a triple negative breast cancer. In one embodiment said female may exhibit adverse side effect by chemotherapeutic agents. Said NK1 receptor antagonist may be e.g. aprepitant or fosaprepitant. It may be administered without or in combination with chemotherapeutic agents as mentioned above e.g. tamoxifen, raloxifene, anastrozole, and/or exemestane.

Subject matter of the invention is a NK-1 receptor antagonist for use in a method of prevention of cancer or a pharmaceutical composition according to the present invention for use in a method of preventing cancer wherein said NK-1 receptor antagonist or said pharmaceutical composition is administered to a subject having not cancer but having an enhanced risk of getting cancer preferably of >3% in 5 years, preferably 10% within 5 years, more preferably 30% within 5 years, more preferably 50% within 5 years, most preferably 80% within 5 years, wherein said subject is selected from one or more of these groups, but not limited to these groups:
a subject having had cancer but has been cured from cancer
a subject having had breast cancer but has been cured from cancer a female subject receiving Hormone Replacement Therapy HRT or having received HRT more than five years
a subject having a genetic predisposition to get cancer, e.g. a female subject having a BRCA mutation
a subject with a family history of getting cancer
a subject with history or prevalence of a disease associated with an elevated risk to develop cancer, such as for instance, such as for instance diabetes
a subject, who uses or used to smoke
a subject with a score-based elevated risk to get cancer
a female subject having an elevated pro-Neurotensin level in a sample of fastening blood a (female) subject having an reduced level of PENK or fragments thereof including MRPENK in a blood sample.

Algorithms (risk scores) have been developed to predict the individual risk to develop various kind of cancers within a certain time period (see for instance http://yourdiseaserisk.wustl.edu/YDRDefault.aspx?ScreenControl=YDRGeneral&ScreenName=YDRCancer_Index, https://www3.mdanderson.org/publicedu/prevention/).

Algorithms have been developed to predict the individual risk to develop breast cancer within a certain time period. One example is the Breast Cancer Risk Assessment Tool (http://www.cancer.gov/bcrisktool/). The Breast Cancer Risk Assessment Tool is based on a statistical model known as the “Gail model,” which is named after Dr. Mitchell Gail, Senior Investigator in the Biostatistics Branch of NCI's Division of Cancer Epidemiology and Genetics. The model uses a woman's own personal medical history (number of previous breast biopsies and the presence of atypical hyperplasia in any previous breast biopsy specimen), her own reproductive history (age at the start of menstruation and age at the first live birth of a child), and the history of breast cancer among her first-degree relatives (mother, sisters, daughters) to estimate her risk of developing invasive breast cancer over specific periods of time. Data from the Breast Cancer Detection Demonstration Project (BCDDP), which was a joint NCI and American Cancer Society breast cancer screening study that involved 280,000 women aged 35 to 74 years, and from NCI's Surveillance, Epidemiology, and End Results (SEER) Program were used in developing the model. Estimates for African American women were based on data from the Women's Contraceptive and Reproductive Experiences (CARE) Study and from SEER data. CARE participants included 1,607 women with invasive breast cancer and 1,637 without. Estimates for Asian and Pacific Islander women in the United States were based on data from the Asian American Breast Cancer Study (AABCS) and SEER data. AABCS participants included 597 Asian and Pacific Islander women with invasive breast cancer, and 966 women without breast cancer.

The Gail model has been tested in large populations of white women and has been shown to provide accurate estimates of breast cancer risk. In other words, the model has been “validated” for white women. It has also been tested in data from the Women's Health Initiative for African American women, and the model performs well, but may underestimate risk in African American women with previous biopsies. The model has been validated for Asian and Pacific Islander women in the WHI and data from SEER.
Gail M H, Brinton L A, Byar D P, Corle D K, Green S B, Shairer C, Mulvihill J J: Projecting individualized probabilities of developing breast cancer for white females who are being examined annually. J Natl Cancer Inst 81(24):1879-86, 1989.
Costantino J P, Gail M H, Pee D, Anderson S, Redmond C K, Benichou J, Wieand H S: Validation studies for models projecting the risk of invasive and total breast cancer incidence. J Natl Cancer Inst 91(18):1541-8, 1999.
Gail M H, Costantino J P, Bryant J, Croyle R, Freedman L, Helzlsouer K, Vogel V: Weighing the risks and benefits of tamoxifen treatment for preventing breast cancer. J Natl Cancer Inst 91(21):1829-46, 1999.
Rockhill B, Spiegelman D, Byrne C, Hunter D J, Colditz G A: Validation of the Gail et al. model of breast cancer risk prediction and implications for chemoprevention. J Natl Cancer Inst 93(5):358-66, 2001.
Gail M H, Costantino J P, Pee D, Bondy M, Newman L, Selvan M, Anderson G L, Malone K E, Marchbanks P A, McCaskill-Stevens W, Norman S A, Simon M S, Spirtas R, Ursin G, and Bernstein L. Projecting Individualized Absolute Invasive Breast Cancer Risk in African American Women. J Natl Cancer Inst 99(23):1782-1792, 2007.
Matsuno R K, Costantino J P, Ziegler R G, Anderson G L, Li H, Pee D, Gail M H. Projecting Individualized Absolute Invasive Breast Cancer Risk in Asian and Pacific Island American Women. J Nati Cancer Inst 2011. doi:10.1093/jnci/djr154

Hormone Replacement Therapy in general refers to any form of hormone therapy wherein the patient, in the course of medical treatment, receives hormones, either to supplement a lack of naturally occurring hormones, or to substitute other hormones for naturally occurring hormones (http://en.wikipedia.org/wiki/Hormone_replacement_therapy). Hormone replacement therapy for menopause is based on the idea that the treatment may prevent discomfort caused by diminished circulating estrogen and progesterone hormones, or in the case of the surgically or prematurely menopausal, that it may prolong life and may reduce incidence of dementia. It involves the use of one or more of a group of medications designed to artificially boost hormone levels. The main types of hormones involved are estrogens, progesterone or progestins, and sometimes testosterone. It often referred to as “treatment” rather than therapy.

A subject having an enhanced risk of cancer may be a subject having an inherited disposition to develop cancer and/or other risk factors, which are known today (such as for instance smoking, another prevalent disease such as for instance diabetes, elevated pro-Neurotensin, lowered pro-Enkephalin) or become known in the future.

Said subject that has an enhanced risk of cancer may be a fasting female subject having an elevated level of pro-neurotensin. A female subject having an enhanced risk of getting cancer may be identified as follows:
determining the level of pro-Neurotensin or fragments thereof of at least 5 amino acids in a bodily fluid obtained from said female subject; and
correlating said level of pro-Neurotensin or fragments thereof with a risk for getting cancer, wherein an elevated level is predictive for an enhanced risk of getting cancer

In one specific embodiment of identifying a female subject having an enhanced risk of cancer said cancer is selected from the group comprising breast cancer, lung cancer, pancreatic cancer and colon cancer. It has been shown that pro-Neurotensin is a powerful and highly significant biomarker for woman for predicting the risk of getting cancer in a female subject that does not suffer from cancer, this has been described in WO2013132089A2. WO2013132089A2 is herein incorporated by reference.

In one specific embodiment of identifying a female subject having an enhanced risk of cancer is a method for predicting the risk of getting cancer in a female subject that does not suffer from cancer in a female subject comprising:
determining the level of pro-Neurotensin 1-117 or fragments thereof of at least 5 amino acids or pro-Neurotensin 1-117 comprising peptides in a bodily fluid obtained from said female subject; and
correlating said level of pro-Neurotensin 1-117 or fragments thereof or pro-Neurotensin 1-117 comprising peptides with the risk of getting cancer, wherein an elevated level is predictive for an enhanced risk of getting cancer and,
wherein said cancer is selected from the group comprising breast cancer, lung cancer, pancreatic cancer and colon cancer.

In a specific embodiment of the invention said cancer is breast cancer.

The term “elevated level” means a level above a certain threshold level. In a specific embodiment the level of pro-Neurotensin is measured with an immunoassay. More specifically an immunoassay is used as described in Ernst et al. (Peptides (2006), (27) 1787-1793). An immunoassay that may be useful for determining the level of pro-Neurotensin or fragments thereof of at least 5 amino acids may comprise the steps as outlined in Example 2. All thresholds and values have to be seen in correlation to the test and the calibration used according to Example 2. A person skilled in the art may know that the absolute value of a threshold might be influenced by the calibration used. This means that all values and thresholds given herein are to be understood in context of the calibration used in herein (Example 2). A human pro-Neurotensin-calibrator is available by JCI-Diagnostics, Berlin, Germany. Alternatively, the assay may also be calibrated by synthetic or recombinant pro-Neurotensin 1-117 or fragments thereof (see also Ernst et. al, 2006).

The threshold for determining the risk of getting breast cancer in a female subject or diagnosing breast cancer in a female subject according to the methods of the present invention is above 78 pmol/1 pro-Neurotensin, preferred 100 pmol/l, more preferred 150 pmol/l. In a specific embodiment said threshold is about 100 pmol/l. These thresholds are related to the above mentioned calibration method. A pro-Neurotensin value above said threshold means that the subject has an enhanced risk of getting cancer or has already cancer.

Pro-neurotensin, Pro-neurotensin 1-117 or fragments thereof may be selected from the following sequences SEQ ID Nos:

(Pro-Neurotensin 1-147)
SEQ ID NO. 1
SDSEEEMKAL EADFLTNMHT SKISKAHVPS WKMTLLNVCS
LVNNLNSPAE ETGEVHEEEL VARRKLPTAL DGFSLEAMLT
IYQLHKICHS RAFQHWELIQ EDILDTGNDK NGKEEVIKRK
IPYILKRQLY ENKPRRPYIL KRDSYYY
(Pro-Neurotensin 1-125 (large neuromedin N))
SEQ ID NO. 2
SDSEEEMKAL EADFLTNMHT SKISKAHVPS WKMTLLNVCS
LVNNLNSPAE ETGEVHEEEL VARRKLPTAL DGFSLEAMLT
IYQLHKICHS RAFQHWELIQ EDILDTGNDK NGKEEVIKR
KIPYIL
(neuromedin N)
SEQ ID NO. 3
KIPYIL
(neurotensin)
SEQ ID NO. 4
pyroQLYENKPRRP YIL
(Pro-Neurotensin 1-117)
SEQ ID NO. 5
SDSEEEMKAL EADFLTNMHT SKISKAHVPS WKMTLLNVCS
LVNNLNSPAE ETGEVHEEEL VARRKLPTAL DGFSLEAMLT
IYQLHKICHS RAFQHWELIQ EDILDTGNDK NGKEEVI
(Pro-Neurotensin 1-132)
SEQ ID NO. 6
SDSEEEMKAL EADFLTNMHT SKISKAHVPS WKMTLLNVCS
LVNNLNSPAE ETGEVHEEEL VARRKLPTAL DGFSLEAMLT
IYQLHKICHS RAFQHWELIQ EDILDTGNDK NGKEEVIKRK
IPYILKRQLY EN
(Pro-Neurotensin 120-140)
SEQ ID NO. 7
KIPYILKRQL YENKPRRPYIL
(Pro-Neurotensin 120-147)
SEQ ID NO. 8
KIPYILKRQL YENKPRRPYIL KRDSYYY
(Pro-Neurotensin 128-147)
SEQ ID NO. 9
QLYENKPRRP YILKRDSYYY

Pro-neurotensin 1-117 or fragments thereof of at least 5 amino acids or pro-Neurotensin 1-117 comprising peptides in a bodily fluid obtained from said female subject that is predictive for the risk of getting cancer in said female subject that does not suffer from cancer is released from the small intestine. It may not be released from cancer cells as the female subject does not suffer from cancer. The release of neurotensin from the small intestine is stimulated by food intake, especially by fat, and is known to regulate gastrointestinal motility and pancreatic and biliary secretion. Pro-neurotensin 1-117 and fragments thereof or pro-Neurotensin 1-117 comprising peptides are used as a surrogate marker for the released neurotensin as neurotensin and pro-Neurotensin 1-117 and fragments thereof or pro-Neurotensin 1-117 comprising peptides are released in equimolar amounts from pro-neurotensin.

In one embodiment of said method of identifying a female subject having an enhanced risk of getting cancer the level of pro-Neurotensin or fragments thereof of at least 5 amino acids or pro-Neurotensin 1-117 comprising peptides in a bodily fluid is the fasting level of pro-Neurotensin or fragments thereof of at least 5 amino acid or pro-Neurotensin 1-117 comprising peptides. Fasting level means no food uptake 12 h prior sampling.

A bodily fluid may be selected from the group comprising blood, serum, plasma, urine, cerebro spinal liquid (csf), and saliva.

In another embodiment said female subject has been diagnosed before with having cancer and has been cured at the time the sample of bodily fluid is taken from said female subject and the risk of re-occurrence of getting cancer is determined or alternatively the re-occurrence of cancer is diagnosed. In a more specific embodiment of said method of identifying a female subject having an enhanced risk of getting cancer the level of pro-Neurotensin 1-117 is determined.

In a specific embodiment the level of pro-Neurotensin is measured with an immunoassay. More specifically an immunoassay is used as described in Ernst et al. (Peptides (2006), (27) 1787-1793). An immunoassay that may be useful for determining the level of pro-Neurotensin or fragments thereof of at least 5 amino acids may comprise the steps as outlined in Example 2. All thresholds and values have to be seen in correlation to the test and the calibration used according to Example 2. A person skilled in the art may know that the absolute value of a threshold might be influenced by the calibration used. This means that all values and thresholds given herein are to be understood in context of the calibration used in herein (Example 2). A human pro-Neurotensin-calibrator is available by ICI-Diagnostics, Berlin, Germany. Alternatively, the assay may also be calibrated by synthetic or recombinant pro-Neurotensin 1-117 or fragments thereof (see also Ernst et. al, 2006).

The threshold for determining the risk of getting breast cancer in a female subject according to the methods of the present invention is above 78 pmol/l pro-Neurotensin, preferred 100 pmol/1, more preferred 150 pmol/l. In a specific embodiment said threshold is about 100 pmol/1. For identifying high-risk subjects the threshold may be 180 pmol/L or 200 pmol/L. These thresholds are related to the above mentioned calibration method. A pro-Neurotensin value above said threshold means that the subject has an enhanced risk of getting cancer.

In one embodiment of the invention said method of identifying a female subject having an enhanced risk of getting cancer is performed more than once in order to monitor the risk of getting breast cancer in a female subject or in order to monitor the course of treatment or prevention. In one specific embodiment said monitoring is performed in order to evaluate the response of said female subject to preventive and/or therapeutic measures taken.

Said subject that has an enhanced risk of cancer may be a female or male subject having a reduced level of Pro-Enkephalin (PENK) or fragments thereof including MRPENK A female or male subject having an enhanced risk of getting cancer may be identified as follows:
determining the level of Pro-Enkephalin (PENK) or fragments thereof including MRPENK, Leu-Enkephalin and Met-Enkephalin of at least 5 amino acids in a bodily fluid obtained from said female subject; and
correlating said level of Pro-Enkephalin or fragments thereof with a risk for getting cancer, wherein a reduced level is predictive for an enhanced risk of getting cancer.

For the method of identifying a female or male subject having an enhanced risk of cancer by using Pro-Enkephalin (PENK) or fragments thereof including MRPENK examples of cancers may be selected from the group comprising breast cancer, lung cancer, pancreatic cancer and colon cancer. In a specific embodiment of said method said cancer is breast cancer. In another specific embodiment said method said cancer is lung cancer.

It should be understood that the term fragments of Pro-Enkephalin also include Leu-Enkephalin and Met-Enkephalin. The method of identifying a female subject having an enhanced risk of cancer by using Pro-Enkephalin (PENK) or fragments thereof including MRPENK is the determination of susceptibility of a woman to acquire cancer, e.g. breast cancer, lung cancer etc The term “reduced level” means a level below a certain threshold level. A bodily fluid may be selected from the group comprising blood, serum, plasma, urine, cerebro spinal liquid (csf), and saliva.

The term “subject” as used herein refers to a living human or non-human organism. Preferably herein the subject is a human subject.

In another embodiment said female or male subject has been diagnosed before with having cancer and has been cured at the time the sample of bodily fluid is taken from said female subject and the risk of reoccurrence of getting cancer is determined or alternatively the re-occurrence of cancer is predicted by using. Pro-Enkephalin (PENK) or fragments thereof including MRPENK and/or Pro-Neurotensin.

Pro-Enkephalin that may be determined in a bodily fluid has the following sequence:

(Pro-Enkephalin (1-243)
SEQ ID NO. 1
ECSQDCATCSYRLVRPADINFLACVMECEGKLPSLKIWETCKELLQLSKP
ELPQDGTSTLRENSKPEESHLLAKRYGGFMKRYGGFMKKMDELYPMEPEE
EANGSEILAKRYGGFMKKDAEEDDSLANSSDLLKELLETGDNRERSHHQD
GSDNEEEVSKRYGGFMRGLKRSPQLEDEAKELQKRYGGFMRRVGRPEWWM
DYQKRYGGFLKRFAEALPSDEEGESYSKEVPEMEKRYGGFMRF

Fragments of Pro-Enkephalin that may be determined in a bodily fluid may be e.g. selected from the group of the following fragments:

(Synenkephalin, Pro-Enkephalin 1-73)
SEQ ID NO. 2
ECSQDCATCSYRLVRPADINFLACVMECEGKLPSLKIWETCKELLQLSKP
ELPQDGTSTLRENSKPEESHLLA
(Met-Enkephalin)
SEQ ID NO. 3
YGGFM
(Leu-Enkephalin)
SEQ ID NO. 4
YGGFL
(ProEnkephalin 90-109)
SEQ ID NO. 5
MDELYPMEPEEEANGSEILA
(Pro Enkephalin 119-159, Mid regional Pro-
Enkephalin-fragment, MRPENK)
SEQ ID NO. 6
DAEEDDSLANSSDLLKELLETGDNRERSHHQDGSDNEEEVS
(Met-Enkephalin-Arg-Gly-Leu)
SEQ ID NO. 7
YGGFMRGL
(Pro-Enkephalin 172-183)
SEQ ID NO. 8
SPQLEDEAKELQ
(Pro-Enkephalin 193-203)
SEQ ID NO. 9
VGRPEWWMDYQ
(Pro-Enkephalin 213-234)
SEQ ID NO. 10
FAEALPSDEEGESYSKEVPEME
(Pro-Enkephalin 213-241)
SEQ ID NO. 11
FAEALPSDEEGESYSKEVPEMEKRYGGFM
(Met-Enkephalin-Arg-Phe)
SEQ ID NO. 12
YGGFMRF

Determining the level of Pro-Enkephalin including Leu-Enkephalin and Met-Enkephalin or fragments thereof may mean that the immunoreactivity towards Pro-Enkephalin or fragments thereof including Leu-Enkephalin and Met-Enkephalin is determined. A binder used for determination of Pro-Enkephalin including Leu-Enkephalin and Met-Enkephalin or fragments thereof depending of the region of binding may bind to more than one of the above displayed molecules. This is clear to a person skilled in the art.

Thus, according to the method of identifying a subject having an enhanced risk of getting cancer the level of immunoreactive analyte by using at least one binder that binds to a region within the amino acid sequence of any of the above peptide and peptide fragments, (i.e. Pro-Enkephalin (PENK) and fragments according to any of the sequences 1 to 12), is determined in a bodily fluid obtained from said subject; and correlated to the specific embodiments of clinical relevance.

In a more specific embodiment of the method of identifying a subject having an enhanced risk of getting cancer the level of MRPENK is determined (SEQ ID NO. 6: Pro-Enkephalin 119-159, Mid regional Pro-Enkephalin-fragment, MRPENK). In a more specific embodiment the level of immunoreactive analyte by using at least one binder that binds to MRPENK is determined and is correlated to the specific embodiments of clinical relevance according to the invention.

Determining the level of Pro-Enkephalin or fragments thereof including Leu-Enkephalin and Met-Enkephalin or fragments thereof may mean that the immunoreactivity towards Pro-Enkephalin or fragments thereof including Leu-Enkephalin and Met-Enkephalin is determined. A binder used for determination of Pro-Enkephalin including Leu-Enkephalin and Met-Enkephalin or fragments thereof depending of the region of binding may bind to more than one of the above displayed molecules. This is clear to a person skilled in the art. In another embodiment of the invention the fragment is not Leu-Enkephalin or Met-Enkephalin. In another embodiment of the invention the immunoreactivity towards Pro-Enkephalin or fragments thereof not including Leu-Enkephalin and Met-Enkephalin is determined.

In a more specific embodiment of the method of identifying a subject having an enhanced risk of getting cancer the level of MRPENK. (SEQ ID NO. 6 (Pro Enkephalin 119-159, Mid regional Pro-Enkephalin-fragment, MRPENK, DAEEDDSLANSSDLLKELLETGDNRERSHHQDGSDNEEEVS) is determined.

In a specific embodiment the level of Pro-Enkephalin or fragments thereof are measured with an immunoassay using antibodies or fragments of antibodies binding to Pro-Enkephalin or fragments thereof. An immunoassay that may be useful for determining the level of Pro-Enkephalin or fragments thereof of at least 5 amino acids may comprise the steps as outlined in Example 3. All thresholds and values have to be seen in correlation to the test and the calibration used according to Example 3, A person skilled in the art may know that the absolute value of a threshold might be influenced by the calibration used. This means that all values and thresholds given herein are to be understood in context of the calibration used in herein (Example 3).

In a specific embodiment the level of Pro-Enkephalin or fragments thereof are measured with an assay using binders selected from the group comprising aptamers, non-Ig scaffolds as described in greater detail below binding to Pro-Enkephalin or fragments thereof.

Binder that may be used for determining the level of Pro-Enkephalin or fragments thereof exhibit an affinity constant to Pro-Enkephalin of at least 10⁷ M⁻¹, preferred 10⁸ M⁻¹, preferred affinity constant is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention. Binding affinity may be determined using the Biacore method, offered as service analysis e.g. at Biaffin, Kassel, Germany (http://www.biaffin.com/de/).

A human Pro-Enkephalin-control sample is available by ICI-Diagnostics, Berlin, Germany http://www.ici-diagnosties.com/. The assay may also be calibrated by synthetic (for our experiments we used synthetic MRPENK, SEQ ID NO. 6) or recombinant Pro-Enkephalin or fragments thereof.

The threshold for determining the risk of getting breast cancer in a female subject according to the methods of identifying the risk of getting cancer is below 100 pmol/1 MRPENK, preferred below 50 pmol/l, more preferred below 40.4 pmol/1. In a specific embodiment said threshold is about 40.4 pmol/1. For identifying high-risk subjects the level may be 44 pmol/L or 35 pmol/L. These thresholds are related to the above mentioned calibration method. A MRPENK value below said threshold means that the subject has an enhanced risk of getting cancer or has already cancer.

An assay that may be used for determining the level of MRPENK is described in Ernst A, Köhrle J, Bergmann A. Peptides. 2006 July; 27(7):1835-40 Ernst, Peptides

In one embodiment of the invention said method is performed more than once in order to monitor the risk of getting breast cancer in a female subject or in order to monitor the course of treatment. In one specific embodiment said monitoring is performed in order to evaluate the response of said female subject to preventive and/or therapeutic measures taken.

Alternatively the level of any of the above analytes may be determined by other analytical methods e.g. mass spectroscopy.

The following subject matter is part of the invention:
1. NK-1 receptor antagonist for use in a method of preventing cancer as a cancer preventing agent.
2. NK-1 receptor antagonist for use in a method of preventing cancer according to claim 1, wherein said NK-1 receptor antagonist is a compound of Formula (I) including tautomers, pharmaceutically acceptable salts thereof or a pharmaceutically acceptable salt of said tautomers:

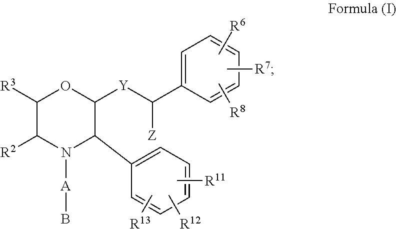

or a tautomer thereof,
or a pharmaceutically acceptable salt thereof or a pharmaceutically acceptable salt of a tautomer thereof, for use in a method of prevention of cancer, wherein
R² and R³ are independently selected from the group consisting of:
(1) hydrogen,
(2) C₁₋₆ alkyl, unsubstituted or substituted with one or more of the substituents selected from:
   (a) hydroxy,
   (b) oxo,
   (c) C₁₋₆ alkoxy,
   (d) phenyl-C₁₋₃ alkoxy,
   (e) phenyl,
   (f) —CN,
   (g) halo,
   (h) —NR⁹R¹⁰, wherein R⁹ and R¹⁰ are independently selected from:
      i. hydrogen,
      ii. C₁₋₆ alkyl,
      iii. hydroxy-C₁₋₆ alkyl, and
      iv. phenyl,
   (i) —NR⁹COR¹⁰, wherein R⁹ and R¹⁰ are as defined above,
   (j) —NR wherein R⁹ and R¹⁰ are as defined above,
   (k) —CONR⁹R¹⁰, wherein R⁹ and R¹⁰ are as defined above,
   (l) —COR⁹, wherein R⁹ is as defined above, and
   (m) —CO₂R⁹, wherein R⁹ is as defined above;
(3) C₂₋₆ alkenyl, unsubstituted or substituted with one or more of the substituent(s) selected from:
   (a) hydroxy,
   (b) oxo,
   (c) C₁₋₆ alkoxy,
   (d) phenyl-C₁₋₃ alkoxy,
   (e) phenyl,
   (f) —CN,
   (g) halo,
   (h) —CONR⁹R¹⁰ wherein R⁹ and R¹⁰ are as defined above,
   (i) —COR⁹ wherein R⁹ is as defined above,
   (j) —CO₂R⁹, wherein R⁹ is as defined above;
(4) C₂₋₆ alkynyl;
(5) phenyl, unsubstituted or substituted with one or more of the substituent(s) selected from:
   (a) hydroxy,
   (b) C₁₋₆ alkoxy,
   (c) C₁₋₆ alkyl,
   (d) C₂₋₅ alkenyl,
   (e) halo,
   (f) —CN,
   (g) —NO₂,
   (h) —CF₃,
   (i) —(CH₂)m-NR⁹R¹⁰, wherein m, R⁹ and R¹⁰ are as defined above,
   (j) —NR⁹COR¹⁰, wherein R⁹ and R¹⁰ are as defined above,
   (k) —NR⁹CO₂R¹⁰, wherein R⁹ and R¹⁰ are as defined above,
   (1) —CONR⁹R¹⁰, wherein R⁹ and R¹⁰ are as defined above,
   (m) —CO₂NR⁹R¹⁰, wherein R⁹ and R¹⁰ are as defined above,
   (n) —COR⁹, wherein R⁹ is as defined above;
   (o) —CO₂R⁹, wherein R⁹ is as defined above;

and, alternatively, the groups R² and R³ are joined together to form a carbocyclic ring selected from the group consisting of:
(a) cyclopentyl,
(b) cyclohexyl,
(c) phenyl,
and wherein the carbocyclic ring is unsubstituted or substituted with one or more substituents selected from:
   (i) C₁₋₆ alkyl,
   (ii) C₁₋₆ alkoxy,
   (iii) —NR⁹R¹⁰, wherein R⁹ and R¹⁰ are as defined above,
   (iv) halo, and
   (v) trifluoromethyl;

and, alternatively, the groups R² and R³ are joined together to form a heterocyclic ring selected from the group consisting of:
(a) pyrrolidinyl,
(b) piperidinyl,
(c) pyrrolyl,
(d) pyridinyl,
(e) imidazolyl,
(f) furanyl,
(g) oxazolyl,
(h) thienyl, and
(i) thiazolyl,
and wherein the heterocyclic ring is unsubstituted or substituted with one or more substituent(s) selected from:
   (i) C₁₋₆ alkyl,
   (ii) oxo,
   (iii) C₁₋₆ alkoxy,
   (iv) —NR⁹R¹⁰, wherein R⁹ and R¹⁰ are as defined above,
   (v) halo, and
   (vi) trifluoromethyl;

R⁶, R⁷ and R⁸ are independently selected from the group consisting of:
(1) hydrogen,
(2) C₁₋₆ alkyl, unsubstituted or substituted with one or more of the substituents selected from:
   (a) hydroxy,
   (b) oxo,
   (c) C₁₋₆ alkoxy,
   (d) phenyl-C₁₋₃ alkoxy,
   (e) phenyl,
   (f) —CN,
   (g) halo,
   (h) —NR⁹R¹⁰, wherein R⁹ and R¹⁰ are as defined above,
   (i) —NR⁹COR¹⁰, wherein R⁹ and R¹⁰ are as defined above,
   (j) —NR⁹CO₂R¹⁰, wherein R⁹ and R¹⁰ are as defined above,
   (k) —CONR⁹R¹⁰, wherein R⁹ and R¹⁰ are as defined above,
   (l) —COR⁹, wherein R⁹ us as defined above, and
   (m) —CO₂R⁹, wherein R⁹ is as defined above;
(3) C₂₋₆ alkenyl, unsubstituted or substituted with one or more of the substituent(s) selected from:
   (a) hydroxy,
   (b) oxo,
   (c) C₁₋₆ alkoxy,
   (d) phenyl-C₁₋₃ alkoxy,
   (e) phenyl,
   (f) —CN,
   (g) halo,
   (h) —CONR⁹R¹⁰, wherein R⁹ and R¹⁰ are as defined above,
   (i) —COR⁹, wherein R⁹ us as defined above,
   (j) —CO₂R⁹, wherein R⁹ us as defined above;
(4) C₂₋₆ alkynyl;
(5) phenyl, unsubstituted or substituted with one or more of the substituents selected from:
   (a) hydroxy,
   (b) C₁₋₆ alkoxy,
   (c) C₁₋₆ alkyl,
   (d) C₂₋₅ alkenyl,
   (e) halo,
   (f) —CN,
   (g) —NO₂,
   (h) —CF₃,
   (i) —(CH₂)ₘ—NR⁹R¹⁰ wherein m, R⁹ and R¹⁰ are as defined above,
   (j) —NR⁹COR¹⁰, wherein R⁹ and R¹⁰ are as defined above,
   (k) —NR⁹CO₂R¹⁰, wherein R⁹ and R¹⁰ are as defined above,
   (l) —CONR⁹R¹⁰, wherein R⁹ and R¹⁰ are as defined above,
   (m) —CO₂NR⁹R¹⁰ wherein R⁹ and R¹⁰ are as defined above,
   (n) —COR⁹, wherein R⁹ is as defined above,
   (o) CO₂R⁹, wherein R⁹ is as defined above;
(6) halo,
(7) —CN,
(8) —CF₃,
(9) —NO₂,
(10) —SR¹⁴, wherein R¹⁴ is hydrogen or C₁₋₅ alkyl,
(11) —SOR¹⁴, wherein R¹⁴ is as defined above,
(12) —SO₂R¹⁴, wherein R¹⁴ is as defined above,
(13) —NR⁹COR¹⁰, wherein R⁹ and R¹⁰ are as defined above,
(14) —CONR⁹COR¹⁰, wherein R⁹ and R¹⁰ are as defined above,
(15) —NR⁹R¹⁰, wherein R⁹ and R¹⁰ are as defined above,
(16) NR⁹CO₂R¹⁰, wherein R⁹ and R¹⁰ are as defined above,
(17) hydroxy,
(18) C₁₋₆ alkoxy,
(19) COR⁹, wherein R⁹ is as defined above,
(20) CO₂R⁹, wherein R⁹ is as defined above,
(21) 2-pyridyl,
(22) 3-pyridyl,
(23) 4-pyridyl,
(24) 5-tetrazolyl,
(25) 2-oxazolyl, and
(26) 2-thiazolyl;

R¹¹, R¹² and R¹³ are independently selected from the definitions of R⁶, R⁷ and R⁸;

A is selected from the group consisting of:
(1) C₁₋₆ alkyl, unsubstituted or substituted with one or more of the substituents selected from:
(a) hydroxy,
(b) oxo,
(c) C₁₋₆ alkoxy,
(d) phenyl-C₁₋₃ alkoxy,
(e) phenyl,
(f) —CN,
(g) halo, wherein halo is fluoro, chloro, bromo or iodo,
(h) —NR⁹R¹⁰, wherein R⁹ and R¹⁰ are as defined above,
(i) —NR⁹COR¹⁰, wherein R⁹ and R¹⁰ are as defined above,
(j) —NR⁹CO₂R¹⁰, wherein R⁹ and R¹⁰ are as defined above,
(k) —CONR⁹R¹⁰, wherein R⁹ and R¹⁰ are as defined above,
(1) COR⁹, wherein R⁹ is as defined above, and (m) —CO₂R⁹, wherein R⁹ is as defined above;
(2) C₂₋₆ alkenyl, unsubstituted or substituted with one or more of the substituent(s) selected from:
   (a) hydroxy,
   (b) oxo,
   (c) C₁₋₆ alkoxy,
   (d) phenyl-C₁₋₃ alkoxy,
   (e) phenyl,
   (f) —CN,
   (g) halo,
   (h) —CONR⁹R¹⁰, wherein R⁹ and R¹⁰ are as defined above,
   (i) —COR⁹, wherein R⁹ is as defined above, and
   (j) CO₂R⁹, wherein R⁹ is as defined above; and
(3) C₂₋₆ alkenyl;

B is a heterocycle, wherein the heterocycle is selected from the group consisting of:

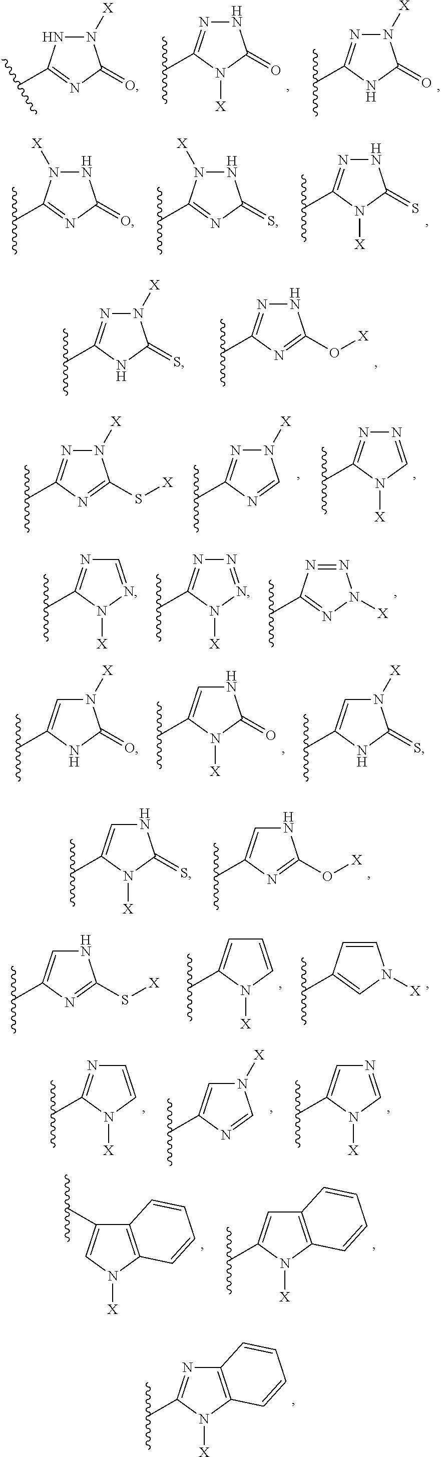

and wherein the heterocycle is substituted in addition to —X with one or more substituent(s) selected from:
(i) hydrogen;
(ii) C₁₋₆ alkyl, unsubstituted or substituted with halo, —CF₃, —OCH₃, or phenyl,
(iii) C₁₋₆ alkoxy,
(iv) oxo,
(v) hydroxy,
(vi) thioxo,
(vii) —SR⁹, wherein R⁹ is as defined above,
(viii) halo,
(ix) cyano,
(x) phenyl,
(xi) trifluoromethyl,
(xii) —(CH₂)ₘ—NR⁹R¹⁰, wherein m is 0, 1 or 2, and R⁹ and R¹⁰ are as defined above,
(xiii) —NR⁹COR¹⁰, wherein R⁹ and R³⁰ are as defined above,
(xiv) —CONR⁹R¹⁰, wherein R⁹ and R¹⁰ are as defined above,
(xv) —CO₂R⁹, wherein R⁹ is as defined above, and
(xvi) —(CH₂)ₘ—OR⁹, wherein m and R⁹ are as defined above;

X is selected from:
(a) hydrogen,
(b) —PO(OH)₂
(c) —PO(OH)O⁻.M⁺, wherein M⁺ is a pharmaceutically acceptable monovalent counterion,
(d) —PO(O⁻)₂.2M⁺,
(e) —PO(O)₂.D²⁺, wherein D²⁺ is a pharmaceutically acceptable divalent counterion,
(f) —CH(R⁴)—PO(OH)O⁻.M⁺, wherein R⁴ is hydrogen or C₁₋₃ alkyl,
(g) —CH(R⁴)—PO(O⁻)₂.2M⁺, and
(h) —CH(R⁴)—PO(O⁻)₂.D²⁺;

Y is selected from the group consisting of:
(1) a single bond,
(2) —O—,
(3) —S—,
(4) —CO—,
(5) —CH₂—,
(6) —CHR¹⁵—, and
(7) —CR¹⁵R¹⁶—, wherein R¹⁵ and R¹⁶ are independently selected from the group consisting of:
   (a) C₁₋₆ alkyl, unsubstituted or substituted with one or more of the substituents selected from:
      (i) hydroxy,
      (ii) oxo,
      (iii) C₁₋₆ alkoxy,
      (iv) phenyl-C₁₋₃ alkoxy,
      (v) phenyl,
      (vi) —CN,
      (vii) halo,
      (viii) —NR⁹R¹, wherein R⁹ and R¹⁰ are as defined above,
      (ix) —NR⁹COR¹⁰, wherein R⁹ and R¹⁰ are as defined above,
      (x) —NR⁹CO₂R¹⁰, wherein R⁹ and R¹⁰ are as defined above,
      (xi) —CONR⁹R¹⁰, wherein R⁹ and R¹⁰ are as defined above,
      (xii) —COR⁹, wherein R⁹ is as defined above, and
      (xiii) —CO₂R⁹, wherein R⁹ is as defined above;
   (b) phenyl, unsubstituted or substituted with one or more of the substituent(s) selected from:
      (i) hydroxy,
      (ii) C₁₋₆ alkoxy,
      (iii) C₁₋₆ alkyl,
      (iv) C₂₋₅ alkenyl,
      (v) halo,
      (vi) —CN,
      (vii) —NO₂,
      (viii) —CF₃,
      (ix) —(CH₂)ₘ—NR⁹R¹⁰, wherein m, R⁹ and R¹⁰ are as defined above,
      (x) —NR⁹COR¹⁰, wherein R⁹ and R¹⁰ are as defined above,
      (xi) —NR⁹CO₂R¹⁰, wherein R⁹ and R¹⁰ are as defined above,
      (xii) —CONR⁹R¹⁰, wherein R⁹ and R¹⁰ are as defined above,
      (xiii) —CO₂NR⁹R¹⁰, wherein R⁹ and R¹⁰ are as defined above,
      (xiv) —COR⁹, wherein R⁹ is as defined above, and
      (xv) —CO₂R⁹, wherein R⁹ is as defined above;

Z is selected from:
(1) hydrogen,
(2) C₁₋₆ alkyl, and
(3) hydroxy, with the proviso that if Y is —O—, Z is other than hydroxy, or if Y is —CHR¹⁵—, then Z and R¹⁵ arc optionally joined together to form a double bond.
3. NK-1 receptor antagonist for use in a method of preventing cancer as a cancer preventing agent according to claims 1 and 2, wherein said NK-1 receptor antagonist is selected from the group comprising a compound of Formula (I) including tautomers, pharmaceutically acceptable salts thereof or a pharmaceutically acceptable salt of said tautomers of Formula (I) and

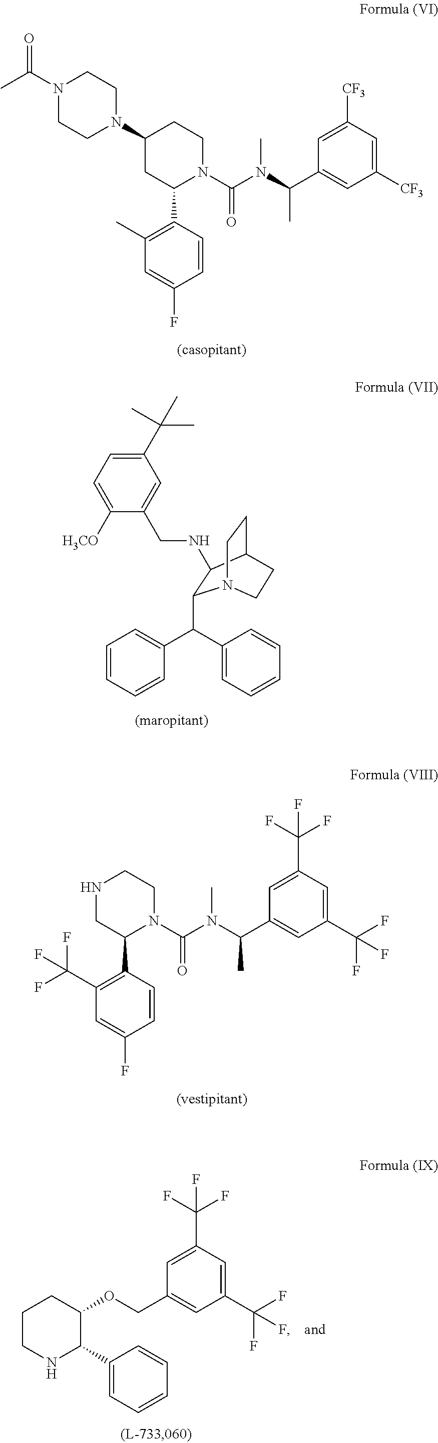 [SMILES: CC(=O)N1CCN([C@H]2CCN(C(=O)N(C)[C@H](C)c3cc(C(F)(F)F)cc(C(F)(F)F)c3)[C@H](c3ccc(F)cc3C)C2)CC1 ; COc1ccc(C(C)(C)C)cc1CNC1C2CCN(CC2)C1C(c1ccccc1)c1ccccc1 ; C[C@H](c1cc(C(F)(F)F)cc(C(F)(F)F)c1)N(C)C(=O)N1CCNC[C@@H]1c1ccc(F)cc1C(F)(F)F ; FC(F)(F)c1cc(CO[C@H]2CCCN[C@H]2c2ccccc2)cc(C(F)(F)F)c1]

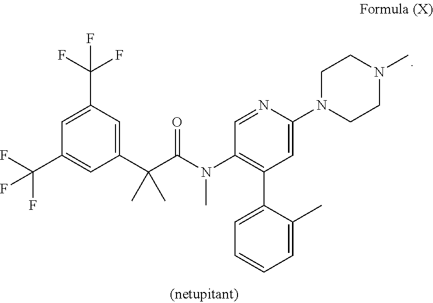 [SMILES: Cc1ccccc1-c1cc(N2CCN(C)CC2)ncc1N(C)C(=O)C(C)(C)c1cc(C(F)(F)F)cc(C(F)(F)F)c1]

including tautomers, pharmaceutically acceptable salts thereof or a pharmaceutically acceptable salt of said tautomers.
4. NK-1 receptor antagonist for use in a method of preventing cancer as a cancer preventing agent according to any of claims 1 to 3, wherein said NK-1 receptor antagonist is a compound of Formula (I) including tautomers, pharmaceutically acceptable salts thereof or a pharmaceutically acceptable salt of said tautomers, wherein:
   R² and R³ are independently selected from the group consisting of:
      (1) hydrogen,
      (2) C₁₋₆ alkyl,
      (3) C₂₋₆ alkenyl, and
      (4) phenyl;
   R⁶, R⁷ and R⁸ are independently selected from the group consisting of:
      (1) hydrogen,
      (2) C₁₋₆ alkyl,
      (3) fluoro,
      (4) chloro,
      (5) bromo,
      (6) iodo, and
      (7) —CF₃;
   R¹¹, R¹² and R¹³ are independently selected from the group consisting of:
      (1) fluoro,
      (2) chloro,
      (3) bromo, and
      (4) iodo;
   A is unsubstituted C₁₋₆ alkyl;
   B is selected from the group consisting of

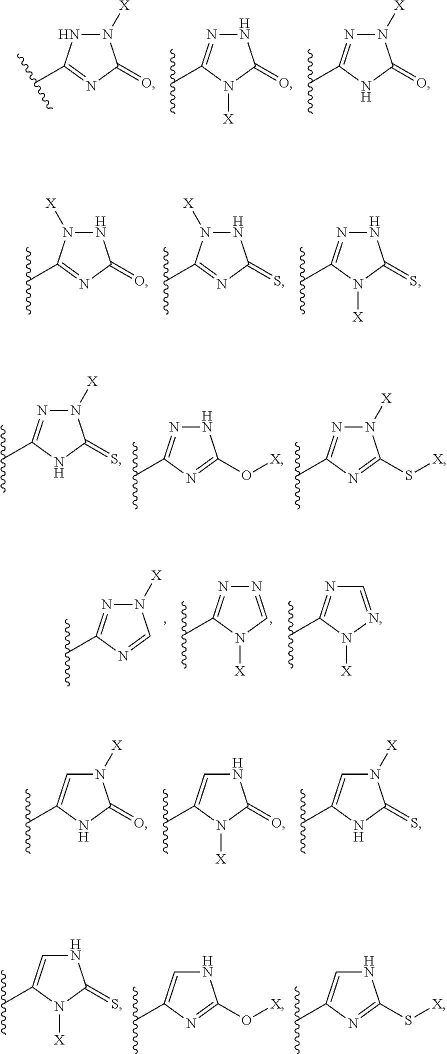

X is selected from:
   (a) hydrogen,
   (b) PO(OH)₂,
   (c) —PO(OH)O⁻.M⁺, wherein M⁺ is a pharmaceutically acceptable monovalent counterion,
   (d) —PO(OH)₂.2M⁺,
   (e) —PO(O⁻)₂.D²⁺, wherein D²⁺ is a pharmaceutically acceptable divalent counterion,
   (f) —CH(R⁴)—PO(OH)O⁻.M⁺, wherein R⁴ is hydrogen or methyl,
   (g) —CH(R⁴)—PO(O⁻)₂.2M⁺, wherein R⁴ is hydrogen or methyl, and
   (h) —CH(R⁴)—PO(O⁻)₂.D²⁺, wherein R⁴ is hydrogen or methyl;
Y is —O—; and
Z is hydrogen or C₁₋₄ alkyl.
5. NK-1 receptor antagonist for use in a method of preventing cancer as a cancer preventing agent according to any of the claims 1 to 4, wherein said NK-1 receptor antagonist is a compound of Formula (I) including tautomers, pharmaceutically acceptable salts thereof or a pharmaceutically acceptable salt of said tautomers, wherein
   Z is C₁₋₄ alkyl.
6. NK-1 receptor antagonist for use in a method of preventing cancer as a cancer preventing agent according to any of the claims 1 to 5, wherein said NK-1 receptor antagonist is a compound of Formula (I) including tautomers, pharmaceutically acceptable salts thereof or a pharmaceutically acceptable salt of said tautomers, wherein Z is —CH₃
7. NK-1 receptor antagonist for use in a method of preventing cancer as a cancer preventing agent according to any of the claims 1 to 6, wherein said NK-1 receptor antagonist is a compound of Formula (I) including tautomers, pharmaceutically acceptable salts thereof or a pharmaceutically acceptable salt of said tautomers, wherein

A is —CH₂— or —CH(CH₃)—.
8. NK-1 receptor antagonist for use in a method of preventing cancer as a cancer preventing agent according to any of the claims 1 to 7, wherein said NK-1 receptor antagonist is a compound of Formula (I) including tautomers, pharmaceutically acceptable salts thereof or a pharmaceutically acceptable salt of said tautomers, wherein
   —B is selected from the group consisting of

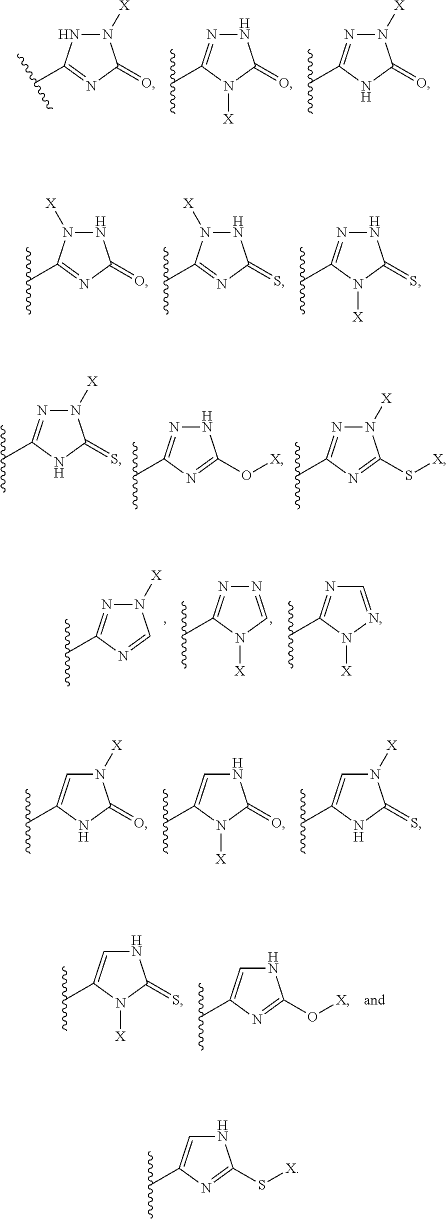

9. NK-1 receptor antagonist for use in a method of preventing cancer as a cancer preventing agent according to any of the claims 1 to 8, wherein said NK-1 receptor antagonist is a compound of Formula (I) including tautomers, pharmaceutically acceptable salts thereof or a pharmaceutically acceptable salt of said tautomers, wherein
   A-B is selected from the group consisting of:

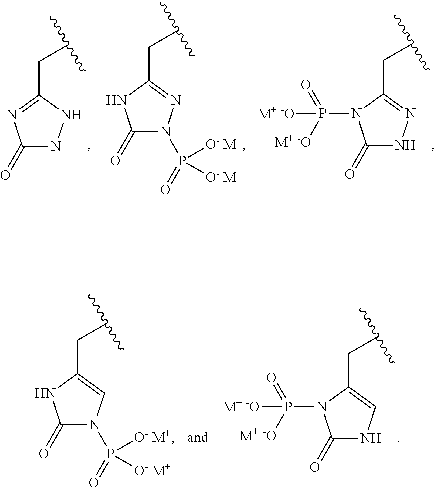

10. NK-1 receptor antagonist for use in a method of preventing cancer as a cancer preventing agent according to any of the claims 1 to 9 wherein said NK-1 receptor antagonist is a compound of Formula (I) including tautomers, pharmaceutically acceptable salts thereof or a pharmaceutically acceptable salt of said tautomers, wherein
   X is selected from the group consisting of:
      (a) hydrogen,
      (b) —PO(011)₂,
      (c) —PO(O⁻)₂.2M⁺, wherein M⁺ is a pharmaceutically acceptable monovalent counterion, and
      (d) —PO(O⁻)₂.D²⁺, wherein D²⁺ is a pharmaceutically acceptable divalent counterion.
11. NK-1 receptor antagonist for use in a method of preventing cancer as a cancer preventing agent according to any of the claims 1 to 10, wherein said NK-1 receptor antagonist is a compound of Formula (I) including tautomers, pharmaceutically acceptable salts thereof or a pharmaceutically acceptable salt of said tautomers, having the structural Formula II:

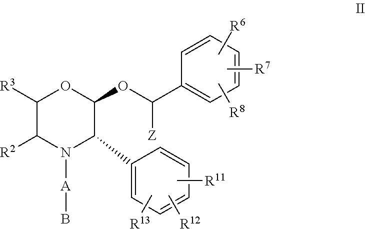

or a pharmaceutically acceptable salt thereof, wherein R², R³, R⁶, R⁷, R⁸, R¹¹, R¹², R¹³, A, B and Z are as defined in claim 2.
12. NK-1 receptor antagonist for use in a method of preventing cancer as a cancer preventing agent according to any of the claims 1 to 11, wherein said NK-1 receptor antagonist is a compound of Formula (I) including tautomers, pharmaceutically acceptable salts thereof or a pharmaceutically acceptable salt of said tautomers, having the structural Formula III:

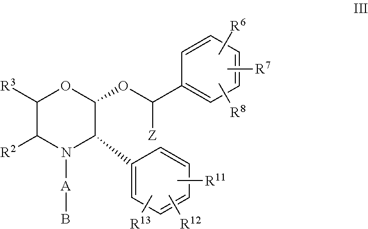

or a pharmaceutically acceptable salt thereof, wherein R², R³, R⁶, R⁷, R⁸, R¹¹, R¹², R¹³, A, B, and Z are as defined in claim 2.
13. NK-1 receptor antagonist for use in a method of preventing cancer as a cancer preventing agent according to any of the claims 1 to 12, wherein said NK-1 receptor antagonist is a compound of Formula (I) including tautomers, pharmaceutically acceptable salts thereof or a pharmaceutically acceptable salt of said tautomers, wherein said compound is selected from the group consisting of:
(1) 2-(R)-(1-(R)-(3,5-bis(trifluoromethyl)phenyl)ethoxy)-3-(S)-(4-fluoro)-phenyl-4-(3-(4-monophosphoryl-5-oxo-1H-1,2,4-triazolo)methyl)morpholine;
(2) 2-(R)-(1-(R)-(3,5-bis(trifluoromethyl)phenyl)ethoxy)-3-(S)-(4-fluoro)-phenyl-4-(3-(1-monophosphoryl-5-oxo-4H-1,2,4-triazolo)methyl)morpholine;
(3) 2-(R)-(1-(R)-(3,5-bis(trifluoromethyl)phenyl)ethoxy)-3-(S)-(4-fluoro)-phenyl-4-(3-(2-monophosphoryl-5-oxo-1H-1,2,4-triazolo)methyl)morpholine;
(4) 2-(R)-(1-(R)-(3,5-bis(trifluoromethyl)phenyl)ethoxy)-3-(S)-(4-fluoro)-phenyl-4-(3-(5-oxyphosphoryl-1H-1,2,4-triazolo)-methyl)morpholine;
(5) 2-(S)-(1-(R)-(3,5-bis(trifluoromethyl)phenyl)ethoxy)-3-(S)-(4-fluoro)-phenyl-4-(3-(1-monophosphoryl-5-oxo-4H-1,2,4-triazolo)methyl)morpholine;
(6) 2-(S)-(1-(R)-(3,5-bis(trifluoromethyl)phenyl)ethoxy)-3-(S)-(4-fluoro)-phenyl-4-(3-(1-monophosphoryl-5-oxo-4H-1,2,4-triazolo)methyl)morpholine
   or a pharmaceutically acceptable salt thereof.
14. NK-1 receptor antagonist for use in a method of preventing cancer as a cancer preventing agent according to any of the claims 1 to 13, wherein said NK-1 receptor antagonist is a compound of Formula (I) including tautomers, pharmaceutically acceptable salts thereof or a pharmaceutically acceptable salt of said tautomers, wherein said compound is:
   2-(R)-(1-(R)-(3,5-bis(trifluoromethyl)phenyl)ethoxy)-3-(S)-(4-fluoro)-phenyl-4-(3-(1-monophosphoryl-5-oxo-4H-1,2,4-triazolo)methyl)morpholine;
   or a pharmaceutically acceptable salt thereof.
15. NK-1 receptor antagonist for use in a method of preventing cancer as a cancer preventing agent according to any of the claims 1 to 14, wherein said NK-1 receptor antagonist is a compound of Formula (I) including tautomers, pharmaceutically acceptable salts thereof or a pharmaceutically acceptable salt of said tautomers, wherein said pharmaceutically acceptable salt is the bis(N-methyl-D-glucamine) salt.
16. NK-1 receptor antagonist for use in a method of preventing cancer as a cancer preventing agent according to any of the claims 1 to 15, wherein said NK-1 receptor antagonist is a compound of Formula (I) including tautomers, pharmaceutically acceptable salts thereof or a pharmaceutically acceptable salt of said tautomers, wherein said compound is:
   2-(R)-(1-(R)-(3,5-bis(trifluoromethyl)phenyl)ethoxy)-3-(S)-(4-fluoro)-phenyl-4-(3-(1-monophosphoryl-5-oxo-4H-1,2,4-triazolo)methyl)morpholine, bis(N-methyl-D-glucamine).
17. NK-1 receptor antagonist for use in a method of preventing cancer according to claim 1, wherein said NK-1 receptor antagonist is selected from the group consisting of:

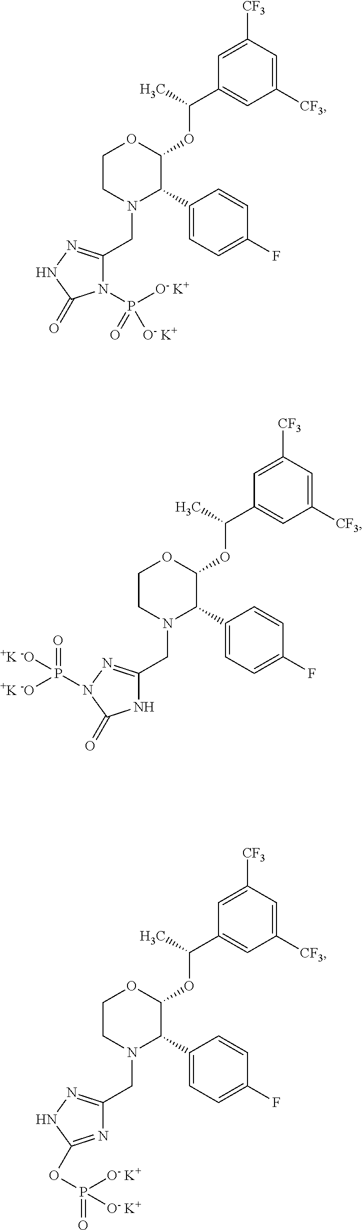

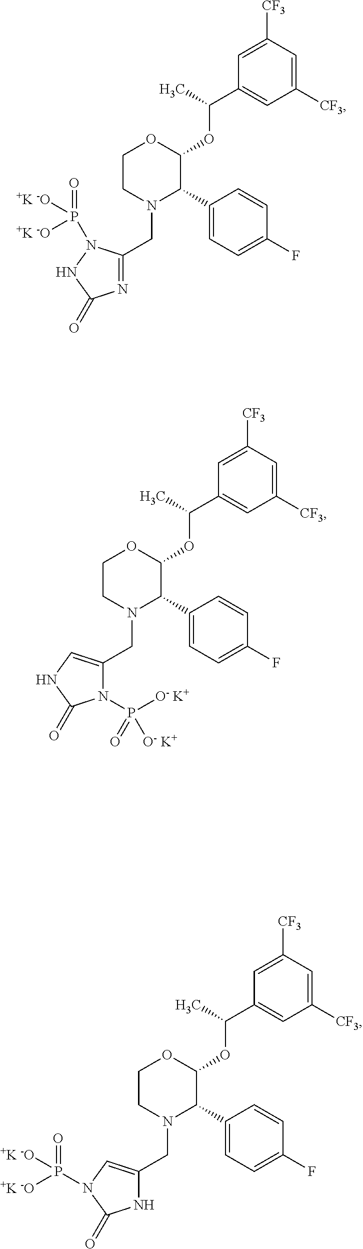

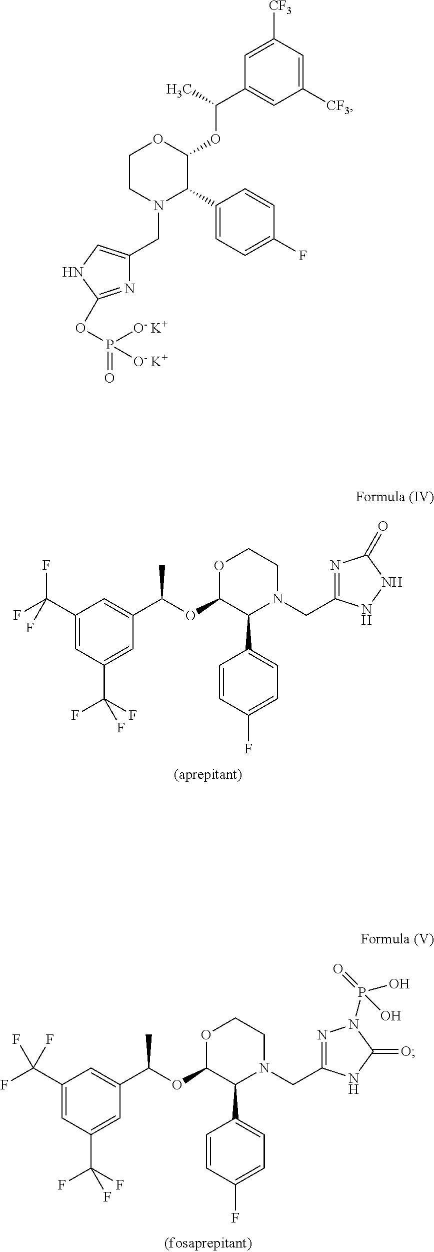

including tautomers thereof, pharmaceutically acceptable salts thereof or a pharmaceutically acceptable salt of said tautomers,
wherein K⁺ is a pharmaceutically acceptable monovalent counterion.
18. NK-1 receptor antagonist for use in a method of preventing cancer according to claim 17, wherein is N-methyl D-glucamine.
19. NK-1 receptor antagonist for use in a method of preventing cancer according to claims 1 to 18 wherein said NK-1 receptor antagonist is selected from a group consisting of:

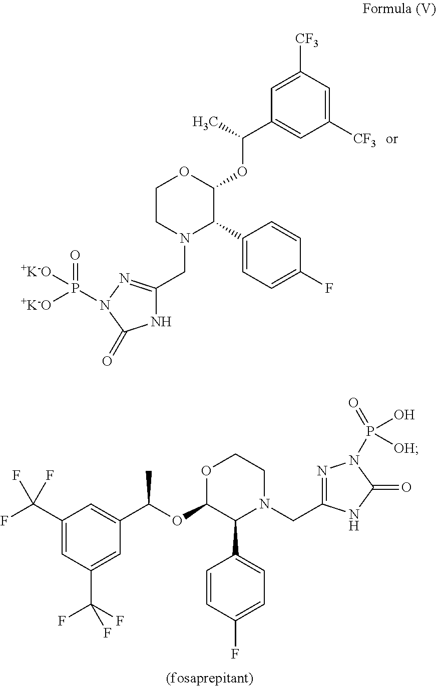

including tautomers thereof, pharmaceutically acceptable salts thereof or a pharmaceutically acceptable salt of said tautomers,
wherein K⁺ is a pharmaceutically acceptable monovalent counterion.
20. NK-1 receptor antagonist for use in a method of preventing cancer according to claim 18, wherein K⁺ is N methyl-D-glucamine.
21. NK-1 receptor antagonist for use in a method of prevention of cancer according to the claims 1 to 18, wherein said NK-1 receptor antagonist is aprepitant including tautomers thereof, pharmaceutically acceptable salts thereof or a pharmaceutically acceptable salt of said tautomers.
22. NK-1 receptor antagonist for use in a method of prevention of cancer according to the any of the preceding claims, wherein said NK-1 receptor antagonist is selected from the group comprising Formula (I) including tautomers, pharmaceutically acceptable salts thereof or a pharmaceutically acceptable salt of said tautomers of Formula (I), and

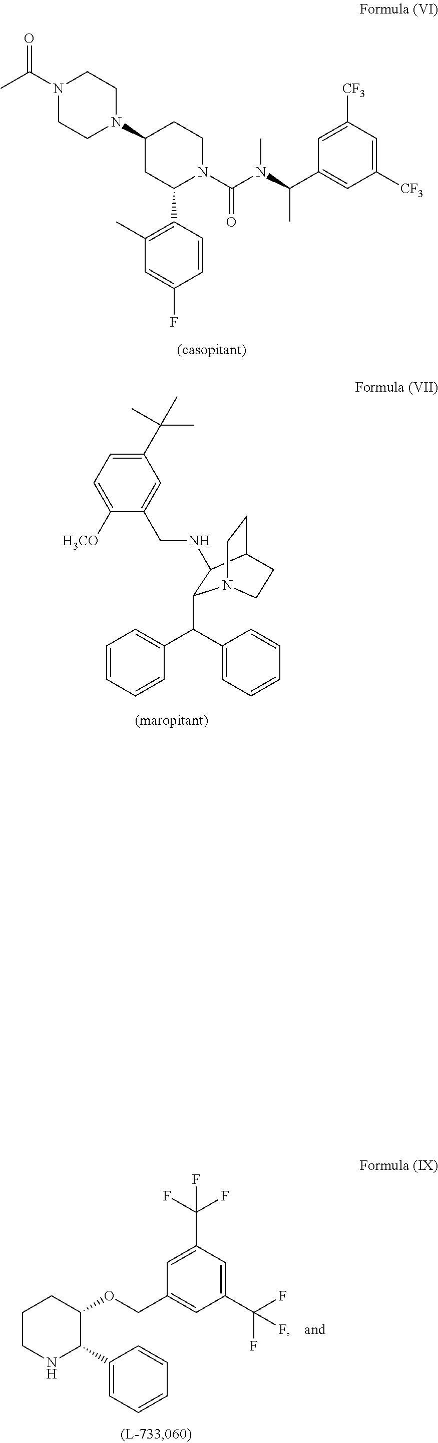 [SMILES: CC(=O)N1CCN([C@H]2CCN(C(=O)N(C)[C@H](C)c3cc(C(F)(F)F)cc(C(F)(F)F)c3)[C@H](c3ccc(F)cc3C)C2)CC1 ; COc1ccc(C(C)(C)C)cc1CNC1C2CCN(CC2)C1C(c1ccccc1)c1ccccc1 ; FC(F)(F)c1cc(CO[C@H]2CCCN[C@H]2c2ccccc2)cc(C(F)(F)F)c1]

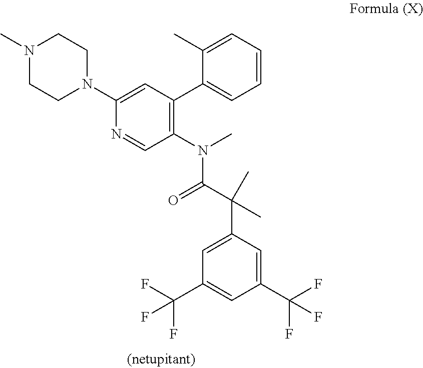 [SMILES: Cc1ccccc1-c1cc(N2CCN(C)CC2)ncc1N(C)C(=O)C(C)(C)c1cc(C(F)(F)F)cc(C(F)(F)F)c1]

and wherein said NK-1 receptor antagonist is administered orally.
23. NK-1 receptor antagonist for use in a method of prevention of cancer according to any of the preceding claims, wherein said NK-1 receptor antagonist is aprepitant including tautomers, pharmaceutically acceptable salts thereof or a pharmaceutically acceptable salt of said tautomers and wherein said NK-1 receptor antagonist is administered orally.
24. NK-1 receptor antagonist for use in a method of prevention of cancer according to claim 22 or 23 wherein the dosage level of said NK-1 receptor antagonist is in an effective amount of 2 to 200 mg/kg per day.
25. NK-1 receptor antagonist for use in a method of prevention of cancer according to the claims 21 to 23 for, wherein said NK-1 receptor antagonist is administered orally to a patient over a period of 2 days to 1 month.
26. NK-1 receptor antagonist for use in a method of prevention of cancer according to the claims 1 to 24, wherein said NK-1 receptor antagonist is selected from the group comprising Formula (I) including tautomers, pharmaceutically acceptable salts thereof or a pharmaceutically acceptable salt of said tautomers of Formula (I),

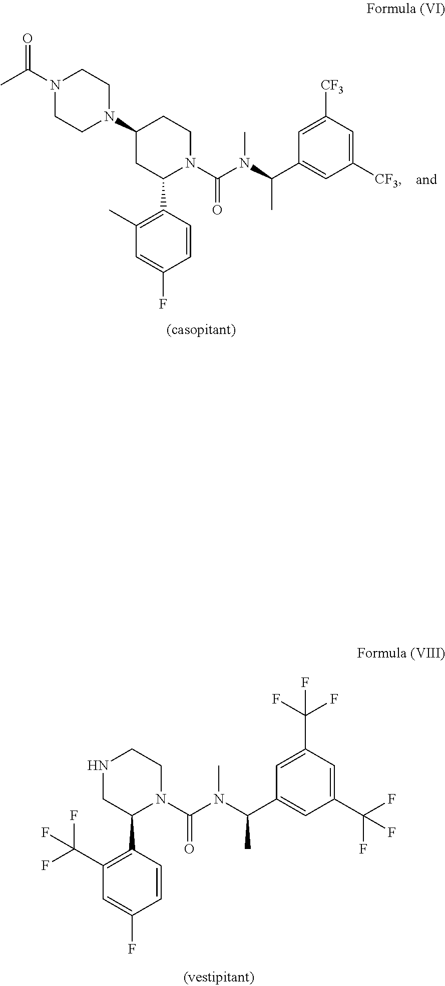 [SMILES: CC(=O)N1CCN([C@H]2CCN(C(=O)N(C)[C@H](C)c3cc(C(F)(F)F)cc(C(F)(F)F)c3)[C@H](c3ccc(F)cc3C)C2)CC1 ; C[C@H](c1cc(C(F)(F)F)cc(C(F)(F)F)c1)N(C)C(=O)N1CCNC[C@@H]1c1ccc(F)cc1C(F)(F)F]

and wherein said NK-1 receptor antagonist is administered intravenously.
27. NK-1 receptor antagonist for use in a method of prevention of cancer according to the claims 1 to 24, wherein said NK-1 receptor antagonist is fosaprepitant including tautomers, pharmaceutically acceptable salts thereof or a pharmaceutically acceptable salt of said tautomers and wherein said NK-1 receptor antagonist is administered intravenously.
28. NK-1 receptor antagonist for use in a method of prevention of cancer according to claim 26 or 28 wherein the dosage level of said NK-1 receptor antagonist is in an effective amount 2 to 200 mg/kg per day or of 60 to 200 mg/kg per day.
29. NK-1 receptor antagonist for use in a method of prevention of cancer according to the claims 26 to 28 for, wherein said NK-1 receptor antagonist is intravenously administered to a patient over a period of 2 days to 1 month.

30. NK-1 receptor antagonist for use in a method of preventing cancer according to any of the claims 1 to 29, wherein said NK-1 receptor antagonist is to be used as monotherapeutic, i.e. not in combination with another anticancer or cancer preventing drug.
31. NK-1 receptor antagonist for use in a method of preventing cancer according to any of the preceding claims, wherein said NK-1 receptor antagonist is not combined with a chemotherapeutic agent.
32. NK-1 receptor antagonist for use in a method of preventing cancer according to any of the preceding claims, wherein said NK-1 receptor antagonist is combined with a chemotherapeutic agent.
33. A pharmaceutical composition for oral administration comprising an effective amount of a NK-1 receptor antagonist according to any of the preceding claims of 200 mg to 10 g.
34. A pharmaceutical composition for intravenous administration comprising an effective amount of a NK-1 receptor antagonist according to any of the preceding claims of 200 mg to 10 g.
35. A pharmaceutical composition according to any of the claims 33 to 34 for use in a method of preventing cancer.
36. The pharmaceutical composition according to any of the claims 33 to 35 for use in a method of preventing cancer wherein said pharmaceutical composition is to be used as monotherapeutic, i.e. not in combination with another anticancer or cancer preventing drug.
37. The pharmaceutical composition according to any of the claims 33 to 35 for use in a method of preventing cancer wherein said pharmaceutical composition is to be used in combination with another anticancer or cancer preventing drug.
38. The pharmaceutical composition according to claim 37 for use in a method of preventing cancer wherein said another anticancer or cancer preventing drug is a chemotherapeutic agent.
39. The pharmaceutical composition according to the claims 33 to 38 for use in a method of preventing cancer, wherein said pharmaceutical composition is administered to a patient over a period of 1 month or of 2 weeks, or of 1 week or of 1 day.
40. NK-1 receptor antagonist for use in a method of prevention of cancer according to the claims 1 to 32 or a pharmaceutical composition according to any of the claims 33 to 39 for use in a method of preventing cancer wherein said NK-1 receptor antagonist or said pharmaceutical composition is administered to a subject having not cancer but having an enhanced risk of getting cancer preferably of >3% in 5 years, preferably 10% within 5 years, more preferably 30% within 5 years, more preferably 50% within 5 years, most preferably 80% within 5 years, wherein said subject is selected from but not restricted to these groups:
   a subject having had cancer but has been cured from cancer
   a female subject receiving Hormone Replacement Therapy
   a subject having a genetic predisposition to get cancer, e.g. a female subject having a BRCA mutation
   a subject with a family history of getting cancer
   a subject with history or prevalence of a disease associated with an elevated risk to develop cancer, such as for instance, such as for instance diabetes
   a subject, who uses or used to smoke
   a subject with a score-based elevated risk to get cancer
   a female subject having an elevated pro-Neurotensin level in a sample of fastening blood
   a (female) subject having an reduced level of PENK or fragments thereof including MRPENK in a blood sample.
41. NK-1 receptor antagonist for use in a method of prevention of cancer according to the claims 1 to 32 or a pharmaceutical composition according to any of the claims 33 to 39 for use in a method of preventing cancer wherein said NK-1 receptor antagonist or said pharmaceutical composition is administered to a subject having not cancer but having an enhanced risk of getting cancer, wherein said subject is a female subject and wherein a female subject having an enhanced risk of getting cancer may be identified as follows:
   determining the level of pro-Neurotensin or fragments thereof of at least 5 amino acids in a bodily fluid obtained from said fasting female subject; and
   correlating said level of pro-Neurotensin or fragments thereof with a risk for getting cancer, wherein an elevated level is predictive for an enhanced risk of getting cancer.
42. NK-1 receptor antagonist for use in a method of prevention of cancer according to the claims 1 to 32 or a pharmaceutical composition according to any of the claims 33 to 40 for use in a method of preventing cancer wherein said NK-1 receptor antagonist or said pharmaceutical composition is administered to a subject having not cancer but having an enhanced risk of getting cancer, wherein said subject having an enhanced risk of getting cancer may be identified as follows:
   determining the level of Pro-Enkephalin (PENK) or fragments thereof including MRPENK Leu-Enkephalin and Met-Enkephalin of at least 5 amino acids in a bodily fluid obtained from said female subject; and
   correlating said level of Pro-Enkephalin or fragments thereof with a risk for getting cancer, wherein a reduced level is predictive for an enhanced risk of getting cancer.

The below definitions apply throughout the specification and the claims.

The terms “tautomer or tautomers” with the context of the invention denote isomers (or constitutional isomers) of the organic compounds disclosed herein that interconvert by chemical reaction commonly referenced as “tautomerization”. This reaction results in the formal migration of a hydrogen atom or proton, accompanied by switch of a single bond and adjacent double bond. The concept of tautomerization is called, “tautomerism”. Due to the rapid interconversion, tautomers are generally considered to be the same chemical compound. Thus, tautomerism is a special case of structural isomerism.

The expressions “valence, valency” denote the number of valence bonds a given atom has formed, or can form, with one or more other atoms. For most elements the number of bonds can vary. The IUPAC definition limits valence to the maximum number of univalent atoms that may combine with the atom that is the maximum number of valence bonds that is possible for the given element. The valence of an element depends on the number of valence electrons that may be involved in the forming of valence bonds.

The expression “monovalent or univalent counterion” denotes an ion or group that has a valence of one and thus can form one covalent bond. By contrast, a divalent molecular entity has a valence of two and can form two sigma bonds to two different atoms or one sigma bond plus one pi bond to a single atom. Alkyl groups and hydroxyl ions are univalent examples; oxo ligands are divalent.

The expression “divalent counterion” denotes a bivalent ion or molecule that has a valence of two and thus can form two bonds with other ions or molecules. In this regard, divalent anions are atoms or radicals with two additional electrons when compared to their elemental state (that is, with 2 more electrons than protons). For instance, S2 is the sulfide anion. By contrast, a divalent cation is missing two electrons as compared with the neutral atom. For instance, iron(II) or Fe²⁺ is a divalent cationic form of iron. Divalent cations are present in abundance in hard water, for example, calcium (Ca²⁺) and magnesium (Mg²⁺).

“Structure-activity relationship (SAR)” is directed to three essential elements, which reflect the interactivity of a ligand (here an NK-1 receptor antagonist) with the NK-1 receptor. First, the ion-pair site interactivity with the bridgehead nitrogen; second, the accessory binding site interactivity with the benzhydryl group; and third, the specific site interactivity with the (2-methoxybenzyl) amino side chain.

The terms “intranvenous; intravenous therapy; i.v. therapy; i.v. injection” with the context of the invention denote the infusion of liquid substances directly into a vein. Intravenous therapy can be used for chemotherapy (the basic treatment for any kind of cancer.) Compared with other routes of administration the intravenous route is the fastest way to deliver fluids and medications throughout the body.

The term “bioavailability” with the context of the present invention denotes a subcategory of absorption and is the fraction of an administered dose of unchanged drug that reaches the systemic circulation, one of the principal pharmacokinetic properties of drugs. By definition, when a medication is administered intravenously, its bioavailability is 100%.

The expression “first line treatment” denotes the initial, or first treatment recommended for a disease or illness by a physician skilled in the medical field. First line treatment is usually the standard treatment (the “gold standard”) given when someone is diagnosed with a particular disease or condition. For instance in case of cancer, chemotherapy is the first line treatment.

### EXAMPLES

### Example 1

### Study

The aim of the herein described study was a) to assess the potential anti-proliferative effect of fosaprepitant and aprepitant in an in vitro cell culture system employing several cancer cell lines, and b) to assess the ability of fosaprepitant and aprepitant to prevent tumor formation in xenograft models for breast cancer, lung cancer and colon cancer (tumor growth inhibition study).

Materials and Methods

Cell Culture System

Aprepitant (Biorbit) and fosaprepitant (IVEMEND®, MSD) were dissolved at a concentration of 100 mM in 100% DMSO (aprepitant) or in 0.9% NaCl (fosaprepitant). Actinomycin D was used as reference compound.

Stock solutions were diluted covering the final concentration range between 0 and 200 μM. The appropriate solvent controls were performed in parallel.

Compounds dissolved in DMSO (aprepitant as well as Actinomycin D) were diluted in 8 semilog steps in DMSO to obtain a dilution series with the 1000 fold final concentration. This series was then prediluted 1/62.5 in medium and finally 10 μL were added to 150 μL of soft agar (0.1% DMSO final concentration).

Fosaprepitant was diluted in 8 semilog steps in 0.9% NaCl to obtain a dilution series with the 1000 fold final concentration. This series was then prediluted 1/62.5 in medium and finally 10 μL were added to 150 μL of soft agar.

Cancer cells stemming from established cancer cell lines (A549, HCT116, MDA-MB231) were cultured in DMEM containing 10% FCS and Penicillin/Streptomycin.

“A549 cells” are adenocarcinomic human alveolar basal epithelial cells. This cell line was first established through the removal and culturing of cancerous lung tissue in the explanted tumor of a 58-year-old male. In nature, these cells are squamous and responsible for the diffusion of some substances, such as water and electrolytes, across the alveoli of lungs. In case said A549 cells are cultured in vitro, they grow as monolayer cells, adhered to the culture flask. A further characteristic is that these cells are able to synthesize lecithin and contain high level of desaturated fatty acids. The A549 cell line is widely used as in vitro model for a type II pulmonary epithelial cell model for drug metabolism and as a transfection host.

“HCT116” cell line denotes human colon cancer cells. These epithelial cells have adherent culture properties, and stem from a male adult. This cell line is a suitable transfection host. This line has a mutation in codon 13 of the ras proto-oncogene, and can be used as a positive control for PCR assays of mutation in this codon.

“MDA-MB23 I” cell line denotes human breast adeno carcinoma cells having epithelial morphology. These cells were isolated from pleural effusions of Caucasian breast cancer patient.

For each cell line 96 well suspension cell culture plates were prepared. 100 μL of the soft agar bottom layer (0.6% final concentration in complete medium) was poured and left to solidify. 50 of the soft agar top layer (0.4% final concentration) containing the corresponding cells and cell number were then added on top, solidified and such 96 well plates incubated overnight at 37° C., 10% CO₂.

Next day, compounds were added into the inner wells of the plate. Subsequently, the assays were incubated in cell culture incubators. Finally, the assays were developed using Alamar Blue and upon 3-5 h of incubation at 37° C. fluorescence intensity was determined (excitation: 560 nm; emission: 590 nm). As low control, cells were treated with 10⁻⁵ M Staurosporine (6-fold values). As high control, cells were treated with 0.1% DMSO (solvent control, 6-fold values).

Raw data were converted into percent soft agar growth relative to high controls (solvent 0.1% DMSO) and low controls (10⁻⁵ M Staurosporine), which were set to 100% and 0%, respectively. IC₅₀ calculation was performed using GraphPad Prism 5 software with a variable slope sigmoidal response-fitting model using 0% soft agar growth as bottom constraint and without bottom constraint and 100% soft agar growth as top constraint.

Xenograft Tumor Models

The ability of fosaprepitant and aprepitant to prevent tumor formation in xenograft models for breast cancer, lung cancer and colon cancer was investigated.

Monolayer MDA-MB-231 cells, HCT-116 cells and A549 cells, respectively, were grown in DMEM+10% FCS. The cells were cultured in a humidified atmosphere of 90% air and 10% carbon dioxide at 37° C. Media was routinely changed every 3 days. Confluent cultures were split 1:3 to 1:3 every 3-4 days using Trypsin/EDTA and seeded at a density of approximately 3-4×10⁶ cells/15 cm²+25 mL medium.

Female BALB/c nude (CAnN.Cg-Foxn1^{nu}/Crl) Mice (Charles River GmbH, Sulzfeld, Germany) aged 4-5 weeks at delivery and weighing approximately 15-18 g were kept under optimum hygienic conditions, air-conditioned with 10-15 air changes per hour, and continually monitored environment with target ranges for temperature 22±3° C. and for relative humidity 30-70%, 12 hours artificial fluorescent light/12 hours dark. Maximum 4 animals were kept per individual ventilated cage (IVC) and fed with a diet consisting of M-Zucht (ssniff Spezialdiáten GmbH) and autoclaved community tab water.

After an acclimatization period of 4 days (day −2) application of fosaprepitant, aprepitant and vehicle was started: Fosaprepitant was administered intravenously (i.v.) once daily for 20 days, and aprepitant was administered per oral gavage (p.o.) once daily for 20 days. Fosaprepitant was dissolved in 0.9% NaCl and 0.9% NaCl was used analogous to fosaprepitant as control (vehicle 1). As aprepitant is not water-soluble, it was provided as a suspension of ground EMEND® tablets in OraPlus® (Paddock Laboratories, Minneapolis, USA; contains: purified water, microcrystalline cellulose, carboxymethylcellulose sodium, xanthan gum, flavouring, citric acid, sodium phosphate, simethicone, methylparaben, and potassium sorbate, pH 4.2) and OraPlus® was used analogous to aprepitant as control (vehicle 2). For details see Table 1.

TABLE 1
Overview on compounds and vehicles investigated

| | | | | | | Animal |
|---|---|---|---|---|---|---|
| Group | Brand name | Conc. | Route | Scheme | Matrix | Number |
| Vehicle | — | — | i.v. | 1x | 0.9% NaCl | 10 |
| (Fosaprepitant Control) | | | | daily | | |
| Fosaprepitant | IVEMEND ® | 60 mg/kg | i.v. | 1x | 0.9% NaCl | 10 |
| | | | | daily | | |
| Vehicle | — | — | p.o. | 1x | OraPlus ® | 10 |
| (Aprepitant Control) | | | | daily | | |
| Aprepitant | EMEND ® | 100 mg/kg | p.o. | 1x | OraPlus ® | 10 |
| | | | | daily | | |

Two days following the start of compound application (day 0), 5×10⁶ tumor cells (MDA-MB-231, HCT-116 and A549, respectively) in 100 μL PBS buffer were implanted subcutaneously into the left flank of the female BALB/c nude mice using a 29 G needle syringe. In the following, animal weights were measured (balance: Mettler Toledo PB602-L). Primary tumor sizes were measured by callipering (manual calliper, OMC Fontana). Tumor sizes were calculated according to the formula W²×L/2 (L=length and W=the perpendicular width of the tumor, L>W).

Animal behavior and welfare was observed daily and tumor growth was recorded every two days by calliper measurement over a period of 30 days.

Results

a) Cell culture system in vitro

In the cell culture system, growth of three cancer cell lines (A549; HCT116; MDA-MB231) was assessed depending on various doses of aprepitant (FIG. 1) and fosaprepitant (FIG. 2) applied. IC₅₀ values were determined using standard parameters based on the signal of the solvent control as Top constraint (100% Soft agar growth) and the signal of the Staurosporine control as Bottom constraint (0% Soft agar growth). The respective IC₅₀ values are summarized in Table 2.

TABLE 2
IC₅₀ values for aprepitant and fosaprepitant treatment

| | Tissue | Incubation | IC₅₀ | | IC₅₀ | |
|---|---|---|---|---|---|---|
| Cell Line | source | time | Aprepitant | | Fosaprepitant | |
| A549 | Lung | 8 days | 3.6 | μM | 1.7 | μM |
| HCT116 | Colon | 8 days | 3.4 | μM | 3 | μM |
| MDA-MB231 | Breast | 11 days | 17 | μM | 36 | μM |

Both aprepitant and fosaprepitant had an antiproliferative effect on the three cell lines tested.

b) Xenograft tumor models

In the xenograft cancer models the preventive application of either aprepitant or fosaprepitant abolished formation of all tumors investigated (Table 3).

TABLE 3
Tumor volumes in three xenograft cancer models depending on preventive application
of aprepitant and fosaprepitant. Tumor volumes are shown as mean in mm³. As values for the
two different vehicles used did not differ, these data were pooled.

| Days after | | | | | | | | | |
|---|---|---|---|---|---|---|---|---|---|
| tumor cell | MDA-MB-231 | | | HCT116 | | | A549 | | |
| inoculation | vehicle | Aprepitant | Fos | Vehicle | Aprepitant | Fos | vehicle | Aprepitant | Fos |
| 10 | 31 | <20 | <20 | 61 | <20 | <20 | 44 | <20 | <20 |
| 20 | 72 | <20 | <20 | 320 | <20 | <20 | 215 | <20 | <20 |
| 30 | 184 | <20 | <20 | 1032 | <20 | <20 | 734 | <20 | <20 |

### Example 2

### Immunoassay for the Quantification of Human Pro-Neurotensin

The technology used was a sandwich coated tube luminescence immunoassay, based on Acridinium ester labelling.

Labelled compound (tracer): 100 μg (100 μl) LA (1 mg/ml in PBS, pH 7.4, was mixed with 10 μl Acridinium NHS-ester (1 mg/ml in acetonitrile, InVent GmbH, Germany) (EP 0353971) and incubated for 20 min at room temperature. Labelled LA was purified by gel-filtration HPLC on Bio-Sil SEC 400-5 (Bio-Rad Laboratories, Inc., USA) The purified LA was diluted in (300 mmol/1 potassiumphosphate, 100 mmol/1 NaCl, 10 mmol/1 Na-EDTA, 5 g/l bovine serum albumin, pH 7.0). The final concentration was approx. 800.000 relative light units (RLU) of labelled compound (approx. 20 ng labeled antibody) per 200 μl. Acridiniumester chemiluminescence was measured by using an AutoLumat LB 953 (Berthold Technologies GmbH & Co. KG).

Solid phase: Polystyrene tubes (Greiner Bio-One International AG, Austria) were coated (18 h at room temperature) with SPA (1.5 μg SPA/0.3 ml 100 mmol/1 NaCl, 50 mmol/1 Tris/HCl, pH 7.8). After blocking with 5% bovine serum albumine, the tubes were washed with PBS, pH 7.4 and vacuum dried.

Calibration:

The assay was calibrated, using dilutions of pro-Neurotensin-containing human serum. A pool of human sera with high pro-Neurotensin-immunoreactivity (InVent Diagostika, Hennigsdorf, Germany) was diluted with horse serum (Biochrom AG, Deutschland) (assay standards).

The standards were calibrated by use of the human Pro-neurotensin-calibrator (ICI-Diagnostics, Berlin, Germany). Alternatively, the assay may be calibrated by synthetic or recombinant pro-Neurotensin 1-117 or fragments thereof (see also Ernst et al., 2006).

Pro-Neurotensin Immunoassay:

50 μl of sample (or calibrator) was pipetted into SPA coated tubes, after adding labeleld LA (200 μl), the tubes were incubated for 16-22 h at 18-25° C. Unbound tracer was removed by washing 5 times (each 1 ml) with washing solution (20 mmol/1 PBS, pH 7.4, 0.1% Triton X-100).

Tube-bound LA was measured by using the LB 953.

### Example 3

### MRPENK Immunoassay

An assay for stable MRPENK (amino acids 119 to 159 of proenkephalin A) has been previously reported in detail (Ernst A, Köhrle J, Bergmann A. Proenkephalin A 119-159, a stable proenkephalin A precursor fragment identified in human circulation. Peptides 2006; 27:1835-40.) and was modified as follows (Ng L L, Sandhu J K, Narayan H, Quinn P A, Squire I B, Davies J E, Bergmann A, Maisel A, Jones D J. J Am Coil Cardiol. 2013 October 8. doi:pii: S0735-1097(13)05557-5. 10.1016/j.jacc.2013.09.037.): in brief, 2 mouse monoclonal anti-PENK antibodies were developed by immunization with PENK peptide (amino acids 119 to 159 of proenkephalin A). One antibody (2 μg) was used to coat polystyrene tubes.

The other antibody labeled with methylacridinium ester served as the detector antibody. Standards (PENK peptide; amino acids 119 to 159 of proenkephalin A) and samples (50 μl) were incubated in tubes with the detector antibody (150 μl). After equilibration, the tubes were washed, and bound chemiluminescence was detected with a luminometer (LB952T/16, Berthold Technologies GmbH & Co., Wildbad, Germany). The lower detection limit of the immunoassay was 5.5 pmol/L. Intra- and interassay coefficients of variation were 6.4% and 9.5% at 50 pmol/L, and 4.0% and 6.5% at 150 pmol/L, respectively. The mean±SEM normal range was 46.6±0.21 pmol/L, with a median (range) of 45 (9 to 518) pmol/L.

### Example 4

### Population Study

Methods

We measured MRPENK in fasting plasma from 2559 female participants of the population based Malmö Diet and Cancer Study baseline exam in 1991-1994 (age 58±6 years and 59% females). We used multivariable adjusted (all traditional cardiovascular risk factors, diabetes risk factors and in analyses of cancer also heredity for cancer) Cox proportional hazards models to relate baseline MRPENK (hazard ratio per each standard deviation increase of log-transformed MRPENK) to the time to the first event of each of the studied endpoints during a median follow-up time of more than 12 years. Endpoints were retrieved through the Swedish National Hospital Discharge Registry, the Swedish Myocardial Infarction Registry, the Stroke in Malmo Registry and the Swedish Cancer Registry. Retrieval of endpoints through these registries has been validated and found to be accurate (see also Belting et al. Cancer Epidemiol Biomarkers Prey; 1-10. 2012 AACR).

Clinical Characteristics of Females in the Study

TABLE 4
Descriptive Statistics

| | N | Mean | Std. Deviation |
|---|---|---|---|
| Age at MDCS screening | 2559 | 57.554 | 5.9403 |
| Systolic blood pressure (mmHg) | 2559 | 140.50 | 19.311 |
| Diastolic blood pressure (mmHg) | 2559 | 85.65 | 9.117 |
| body-mass-index (weight/kg × kg) | 2559 | 25.5196 | 4.19083 |
| WAIST (cm) | 2559 | 76.99 | 10.245 |
| Glucose (mmol/l) | 2559 | 5.0418 | 1.21798 |
| Triglycerides (mmol/l) | 2559 | 1.2245 | .58404 |
| High density lipoprotein (mmol/l) | 2559 | 1.5123 | .36949 |
| Low density lipoprotein (mmol/l) | 2559 | 4.2016 | 1.04762 |
| P-INSULIN | 2512 | 7.223 | 5.4223 |

FIG. 1: frequent distribution of MRPENK in the females population:

The mean value was 47.2 pmol/L, standard deviation=1.2 pmol/L. The x axis is the Logarithmus Naturalis (LN) of the MRPENK concentration. All results were within the measurement of the assay, the lowest MRPENK concentration was 9 pmol/L. These results indicating the suitability of the used assay (assay sensitivity 5.5 pmol/L).

MRPENK and Prediction of Breast Cancer

We assessed the relationship between MRPENK and breast cancer (Table 5). There was a strong relationship between MRPENK and breast cancer in females. In a fully adjusted model each SD increase of MRPENK was associated with a 28.6% risk reduction or each SD of decrease of MRPENK (revPENK) was associated with a 40% increased risk of future breast cancer (table 5) and the top versus bottom quartile of MRPENK identified a more than 3-fold difference in risk of breast cancer (see Table 6 and FIG. 3).

TABLE 5
Variables in the Equation^{o}

| | | | | | | | 95.0% CI | |
|---|---|---|---|---|---|---|---|---|
| | | | | | | | for Exp(B) | |
| | B | SE | Wald | df | Sig. | Exp(B) | Lower | Upper |
| AGE | .007 | .016 | .228 | 1 | .633 | 1.007 | .977 | 1.039 |
| SEX | | | | 0^{a} | | | | |
| BMI_B | .026 | .025 | 1.139 | 1 | .286 | 1.027 | .978 | 1.077 |
| DM_B | −.242 | .407 | .352 | 1 | .553 | .785 | .354 | 1.744 |
| HDL_B | .044 | .252 | .031 | 1 | .660 | 1.045 | .638 | 1.714 |
| LDL_B | −.001 | .090 | .000 | 1 | .988 | .999 | .837 | 1.191 |
| current_smoker | .330 | .195 | 2.888 | 1 | .089 | 1.392 | .950 | 2.037 |
| HER_CANCER_0 | .034 | .176 | .038 | 1 | .846 | 1.035 | .733 | 1.461 |
| LNINS | −.288 | .197 | 2.127 | 1 | .145 | .750 | .509 | 1.104 |
| ZscoreLNPENK_females_noCa | −.337 | .082 | 16.858 | 1 | .000 | .714 | .608 | .839 |

TABLE 6
BREAST CANCER

| | HR per | | | | | | P for |
|---|---|---|---|---|---|---|---|
| | 1 SD | P-value | Quartile 4 | Quartile 3 | Quartile 2 | Quartile 1 | trend |
| Women | 1.40 | <0.001 | 1.0 (ref) | 1.50 | 2.7(1.7-3.4) | 3.6 | <0.001 |
| (2140/ | (13-1.6) | | | (0.81-2.1) | | (2.7-4.9) | |
| 135) | | | | | | | |

Multivariate Cox proportional Hazards models for baseline MRPENK versus incidence of breast cancer.

FIG. 2: Kaplan Meier graphs, illustrating the cumulative breast cancer diagnosis in women Quartile (Q) 1 (below 40.4 pmol/l) quartile 2 (40.4-47.1 pmol/1), quartile 3 (47.2-54.1 pmol/1), quartile 4 (above 54.1 pmol/1). Decreased MRPENK indicates a long term increased risk of breast cancer development. Since any women with cancer history at day of baseline (blood sampling) were excluded, MRPENK is highly predictive for future breast cancer development. Over all, women from Q 1 have a 3.6 times higher risk to develop breast cancer than women from Q 4.

Combination MRPENK and Pro Neurotensin

Since increasing Pro-Neurotensin recently was shown to be highly predictive for breast cancer (Melander O, Maisel A S, Almgren P, Manjer J, Belting M, Hedblad B, Engström G, Kilger U, Nilsson P, Bergmann A, Orho-Melander M. JAMA. 2012 October 10; 308(14):1469-75), we combined both biomarkers for breast cancer prediction.

There was no significant correlation between MRPENK and Pro-Neurotensin (p=0.56). In a combined model using both biomarkers, we found them both independent in breast cancer prediction.

In a fully adjusted model each SD increase of Pro-Neurotensin was associated with a 49.9% risk increase of future breast cancer. Surprisingly, after adding Pro-Neurotensin to the equation, MRPENK was even stronger than without Pro-Neurotensin and showed for each SD increase of MRPENK a 30.8% risk reduction or each SD of decrease of MRPENK (revPENK) was associated with a 44.5% increased risk of future breast cancer (Table 7).

TABLE 7
combined analysis of Pro-Neurotensin and MRPENK for breast cancer prediction.
Variables in the Equation

| | | | | | | | 95.0% CI | |
|---|---|---|---|---|---|---|---|---|
| | | | | | | | for Exp(B) | |
| | B | SE | Wald | df | Sig. | Exp(B) | Lower | Upper |
| AGE | −.003 | .019 | .020 | 1 | .888 | .997 | .960 | 1.036 |
| current_smoker0 | .434 | .204 | 4.505 | 1 | .034 | 1.543 | 1.034 | 2.304 |
| BMI_8 | .001 | .027 | .001 | 1 | .979 | 1.001 | .948 | 1.056 |
| GFR_CG_BSAcorr | −.005 | .008 | .357 | 1 | .550 | .995 | .979 | 1.011 |
| hrt_curr | .730 | .201 | 13.146 | 1 | .000 | 2.075 | 1.399 | 3.079 |
| PNT | .405 | .091 | 19.731 | 1 | .000 | 1.499 | 1.254 | 1.793 |
| PENK | −.368 | .088 | 17.416 | 1 | .000 | .692 | .582 | .823 |

Highest vs. lowest quartile Pro-Neurotensin indicated a 2.56 fold risk for breast cancer development and Pro Enkephalin on top of Pro-Neurotensin lowest vs highest quartile (rev=reversed quartiles Q1=Q4, Q2=Q3, Q3=Q2, Q4=Q1)) an independent 3.6 fold risk (table 9).

Combining highest quartile of Pro-Neurotensin and lowest MRPENK quartile vs. lowest Pro-Neurotensin- and highest MRPENK quartile showed a combined risk of 6.17 (see FIG. 3).

TABLE 8
Table 8: combined analysis of Pro-Neurotensin and MRPENK
for breast cancer prediction.
Variables in Equation

| | | | | | | | 95.0% CI |
|---|---|---|---|---|---|---|---|
| | B | SE | Wald | df | Sig. | Exp(B) | Lower |
| AGE | −.022 | .018 | 1. | 1 | .226 | .978 | .943 |
| current_smoker0 | .391 | .200 | 3.808 | 1 | .051 | 1.478 | .998 |
| hrt_CUT | .652 | .195 | 11.145 | 1 | .001 | 1.920 | 1.309 |
| BMI_B | .012 | .025 | .247 | 1 | .619 | 1.012 | .964 |
| GFR_CG_BSAcorr | −.012 | .008 | 2.279 | 1 | .131 | .988 | .972 |
| NLN_PNT | | | 13.898 | 3 | .003 | | |
| MLN_PNT(1) | .353 | .301 | 1.378 | 1 | .241 | 1.424 | .789 |
| NLN_PNIT(2) | .604 | .286 | 4.452 | 1 | .035 | 1.830 | 1.044 |
| NLN_PNT(3) | .942 | .269 | 12.260 | 1 | .000 | 2.566 | 1.514 |
| Q_PENK_rev | | | 23.361 | 3 | .000 | | |
| Q_PENK_rev(1) | .410 | .331 | 1.534 | 1 | .215 | 1.507 | .787 |
| Q_PENK_rev(2) | .979 | .305 | 10.299 | 1 | .001 | 2.663 | 1.464 |
| Q_PENK_rev(3) | 1.284 | .300 | 18.315 | 1 | .000 | 3.610 | 2.005 |

FIG. 3: Illustration example of combined analysis of MRPENK for breast cancer prediction:

We combined the women with lowest MRPENK (1^{st}) quartile and highest (4^{th}) Pro-Neurotensin quartile (group 3). Within that high risk group about 19.02% of women developed breast cancer within the following 15 years.

Group 2 is a combination of women with 3^{nd} quartile of Pro-Neurotensin and 2^{nd} quartile of MRPENK plus 2^{nd} quartile of Pro-Neurotensin and 3th quartile of MRPENK. Within that medium risk group about 7.48% of women developed breast cancer within the following 15 years.

Group 1 is a combination of women with 1^{st} quartile of Pro-Neurotensin and 4^{th} quartile of MRPENK. Within that low risk group about 3.08% of women developed breast cancer within the following 15 years. The Hazard risk between group 1 and group 3 is about 6.17.

Lung Cancer

MRPENK also predicts lung cancer in females.

40 women developed lung cancer during the observation period. MRPENK is not different in smoking and not smoking women (p=0.44). As expected, smoking is a strong risk prediction marker for lung cancer (p<0.0001). Surprisingly, although smoking is part of the equation, low MRPENK indicated a 3.2 fold risk of developing lung cancer (table 10 a and 10 b).

Table 9 a and 9 b: MRPENK in the prediction of lung cancer in females. The women were grouped in tertiles (see table 10 a) and than analyzed for lung cancer development (see table 10 b). rev=highest tertile (tertile 3), rev (1)=tertile 2 and rev (2)=lowest tertile (tertile 1).

TABLE 9 a
MRPENK[pmol/L]

| Percentile Group of MRPENK | | | |
|---|---|---|---|
| [pmol/L] | Median | Minimum | Maximum |
| 1 | 37.80000 | 9.000 | 42.800 |
| 2 | 47.20000 | 42.900 | 51.300 |
| 3 | 58.30000 | 51.400 | 518.100 |
| Total | 47.25000 | 9.000 | 518.100 |

TABLE 9b
Variables in the Equation

| | B | SE | Wald | df | Sig. | Exp(B) |
|---|---|---|---|---|---|---|
| AGE | .045 | .040 | 1.251 | 1 | .263 | 1.046 |
| current_smoker0 | 1.897 | .427 | 19.761 | 1 | .000 | 6.667 |
| BMI_B | −.034 | .063 | .287 | 1 | .592 | .967 |
| GFR_CG_BSAcorr | −.024 | .019 | 1.592 | 1 | .207 | .976 |
| T_PENK_females_rev | | | 6.698 | 2 | .035 | |
| T_PENK_females_rev(1) | .208 | .580 | .128 | 1 | .721 | 1.231 |
| T_PENK_females_rev(2) | 1.168 | .511 | 5.220 | 1 | .022 | 3.214 |

### Example 5

The preventive application of either aprepitant or fosaprepitant reduced growth of triple negative tumor cells in xenografted nude mice (FIG. 4).

FIG. 4: In vivo treatment of xenografted mice with fosaprepitant and aprepitant.

After an acclimatization period, application of fosaprepitant, aprepitant and vehicle control was started (day −2): Fosaprepitant was administered intravenously (i.v.; 60 mg/kg/d) once daily for 19 days, and aprepitant was administered per oral gavage (p.o.; 100 mg/kg/d) once daily for 19 days. Two days following the start of compound application (day 0), nude mice were xenografted subcutaneously 5×10⁶ tumor cells (MDA-MB-231), as described and separated into control and treatment groups (n=10). The tumor volume was measured and depicted as mm3 at the indicated time points.

### FIGURE DESCRIPTION

FIG. 1. frequence distribution of MRPENK in the females population:

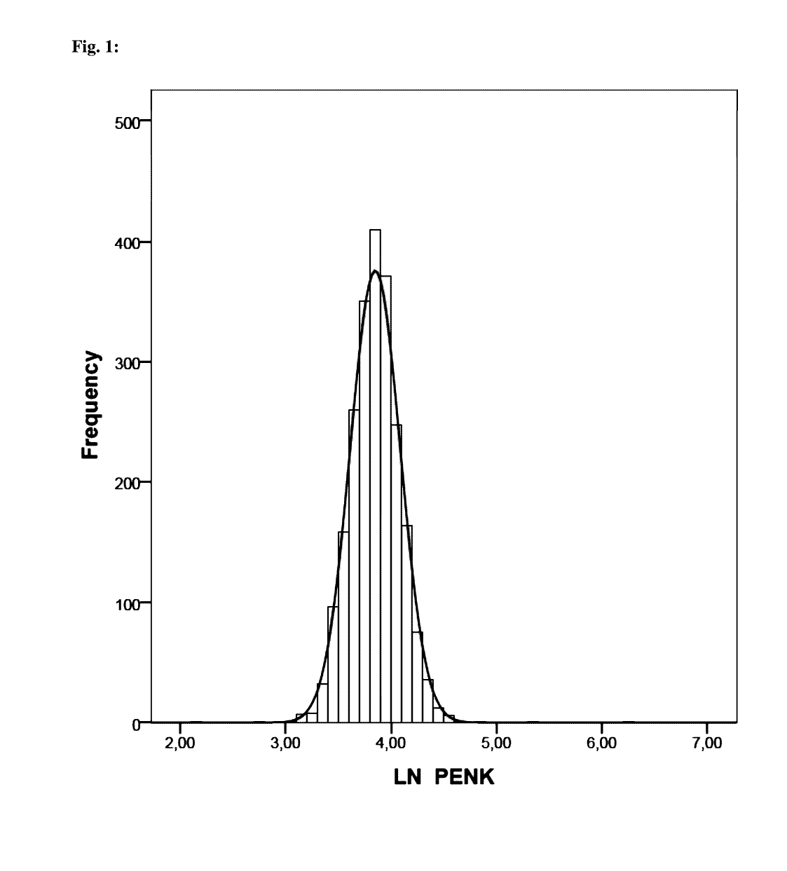

FIG. 2: Kaplan Meier graphs, illustrating the cumulative breast cancer diagnosis in women quartile (Q) 1 (below 40.4 pmol/1) quartile 2 (40.4-47.1 pmol/1), quartile 3 (47.2-54.1 pmol/1), quartile 4 (above 54.1 pmol/1). Decreased MRPENK indicates a long term increased risk of breast cancer development. Since any women with cancer history at day of baseline (blood sampling) were excluded, MRPENK is highly predictive for future breast cancer development.

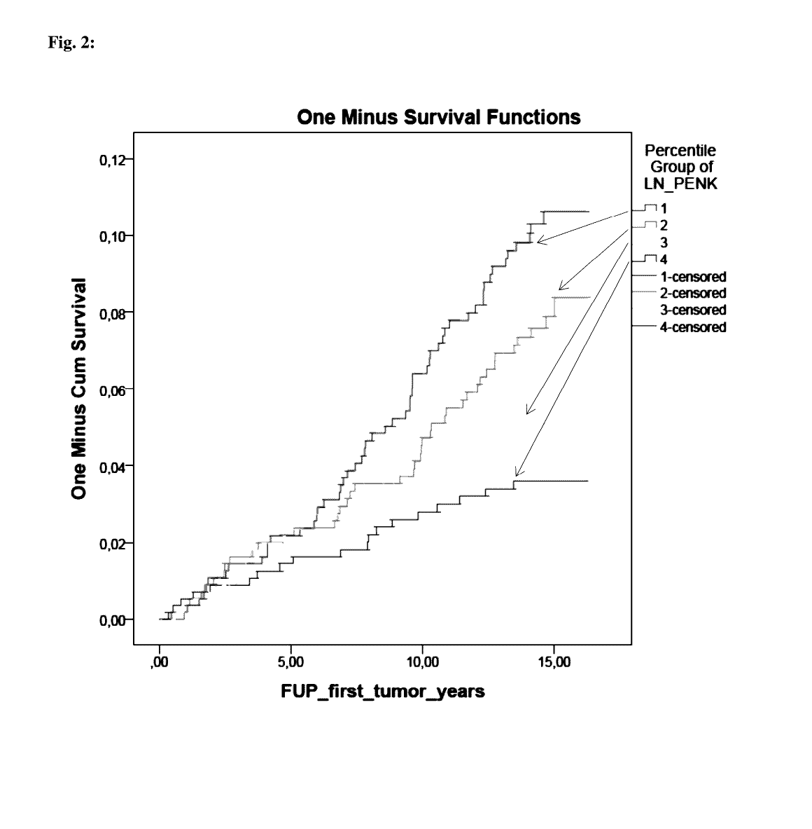

Over all, women from Q 1 have a 3.6 times higher risk to develop breast cancer than women from Q 4.

FIG. 3: Illustration example of combined analysis of MRPENK for breast cancer prediction:

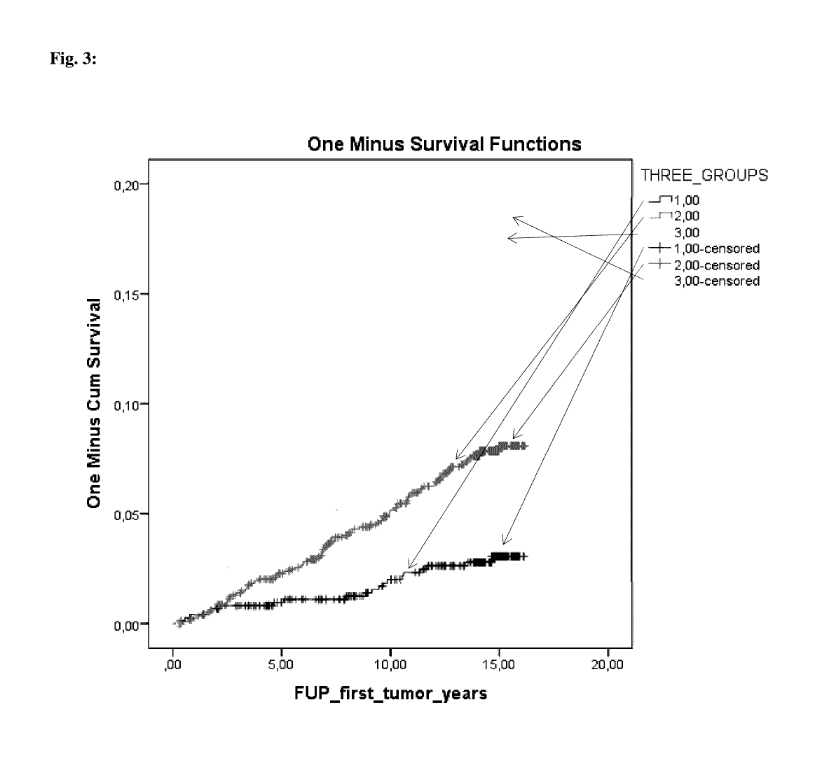

FIG. 4: In vivo treatment of xenografted mice with fosaprepitant and aprepitant. After an acclimatization period, application of fosaprepitant, aprepitant and vehicle control was started (day −2): Fosaprepitant was administered intravenously (i.v.; 60 mg/kg/d) once daily for 19 days, and aprepitant was administered per oral gavage (p.o.; 100 mg/kg/d) once daily for 19 days. Two days following the start of compound application (day 0), nude mice were xenografted subcutaneously 5×10⁶ tumor cells (MDA-MB-231), as described and separated into control and treatment groups (n=10). The tumor volume was measured and depicted as mm3 at the indicated time points.

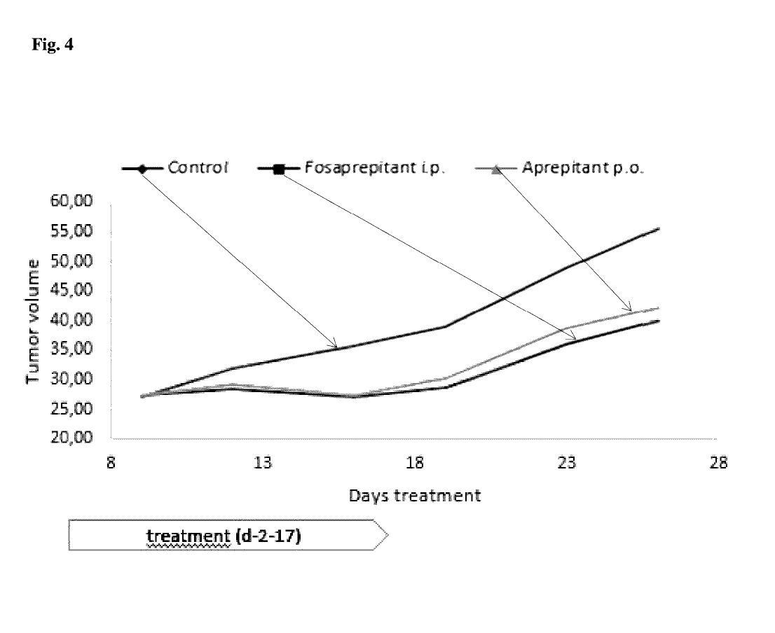

1.-34. (canceled)

35. A method of preventing cancer comprising administering to a subject an amount of NK-1 receptor antagonist effective for use as a cancer preventing agent, wherein said NK-1 receptor antagonist is a compound of Formula (I) including tautomers, pharmaceutically acceptable salts thereof or a pharmaceutically acceptable salt of said tautomers: 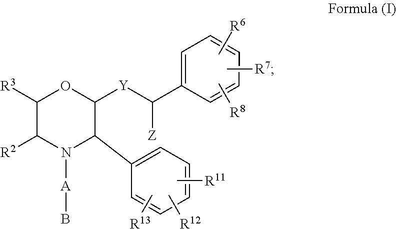
or a tautomer thereof,
or a pharmaceutically acceptable salt thereof or a pharmaceutically acceptable salt of a tautomer thereof, wherein
R² and R³ are independently selected from the group consisting of:
(1) hydrogen,
(2) C₁₋₆ alkyl, unsubstituted or substituted with one or more of the substituents selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —NR⁹R¹⁰, wherein R⁹ and R¹⁰ are independently selected from:
i. hydrogen,
ii. C₁₋₆ alkyl,
iii. hydroxy-C₁₋₆ alkyl, and
iv. phenyl,
(i) —NR⁹COR¹⁰, wherein R⁹ and R¹⁰ are as defined above,
(j) —NR⁹CO₂R¹⁰, wherein R⁹ and R¹⁰ are as defined above,
(k) —CONR⁹R¹⁰, wherein R⁹ and R¹⁰ are as defined above,
(l) —COR⁹, wherein R⁹ is as defined above, and
(m) —CO₂R⁹, wherein R⁹ is as defined above;
(3) C₂₋₆ alkenyl, unsubstituted or substituted with one or more of the substituent(s) selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —CONR⁹R¹⁰ wherein R⁹ and R¹⁰ are as defined above,
(i) —COR⁹ wherein R⁹ is as defined above,
(j) —CO₂R⁹, wherein R⁹ is as defined above;
(4) C₂₋₆ alkynyl;
(5) phenyl, unsubstituted or substituted with one or more of the substituent(s) selected from:
(a) hydroxy,
(b) C₁₋₆ alkoxy,
(c) C₁₋₆ alkyl,
(d) C₂₋₅ alkenyl,
(e) halo,
(f) —CN,
(g) —NO₂,
(h) —CF₃,
(i) —(CH₂)m-NR⁹R¹⁰, wherein m is 0, 1, or 2, and R⁹ and R¹⁰ are as defined above,
(j) —NR⁹COR¹⁰, wherein R⁹ and R¹⁰ are as defined above,
(k) —NR⁹CO₂R¹⁰, wherein R⁹ and R¹⁰ are as defined above,
(l) —CONR⁹R¹⁰, wherein R⁹ and R¹⁰ are as defined above,
(m) —CO₂NR⁹R¹⁰, wherein R⁹ and R¹⁰ are as defined above,
(n) —COR⁹, wherein R⁹ is as defined above;
(o) —CO₂R⁹, wherein R⁹ is as defined above;
and, alternatively, the groups R² and R³ are joined together to form a carbocyclic ring selected from the group consisting of:
(a) cyclopentyl,
(b) cyclohexyl,
(c) phenyl,
and wherein the carbocyclic ring is unsubstituted or substituted with one or more substituents selected from:
C₁₋₆ alkyl,
(ii) C₁₋₆ alkoxy,
(iii) —NR⁹R¹⁰, wherein R⁹ and R¹⁰ are as defined above,
(iv) halo, and
(v) trifluoromethyl;
and, alternatively, the groups R² and R³ are joined together to form a heterocyclic ring selected from the group consisting of:
(a) pyrrolidinyl,
(b) piperidinyl,
(c) pyrrolyl,
(d) pyridinyl,
(e) imidazolyl,
(f) furanyl,
(g) oxazolyl,
(h) thienyl, and
(i) thiazolyl,
and wherein the heterocyclic ring is unsubstituted or substituted with one or more substituent(s) selected from:
(i) C₁₋₆ alkyl,
(ii) oxo,
(iii) C₁₋₆ alkoxy,
(iv) —NR⁹R¹⁰, wherein R⁹ and R¹⁰ are as defined above,
(v) halo, and
(vi) trifluoromethyl;
R⁶, R⁷ and R⁸ are independently selected from the group consisting of:
(1) hydrogen,
(2) C₁₋₆ alkyl, unsubstituted or substituted with one or more of the substituents selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —NR⁹R¹⁰, wherein R⁹ and R¹⁰ are as defined above,
(i) —NR⁹COR¹⁰, wherein R⁹ and R¹⁰ are as defined above,
(j) —NR⁹CO₂R¹⁰, wherein R⁹ and R¹⁰ are as defined above,
(k) —CONR⁹R¹⁰, wherein R⁹ and R¹⁰ are as defined above,
(l) —COR⁹, wherein R⁹ us as defined above, and
(m) —CO₂R⁹, wherein R⁹ is as defined above;
(3) C₂₋₆ alkenyl, unsubstituted or substituted with one or more of the substituent(s) selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —CONR⁹R¹⁰, wherein R⁹ and R¹⁰ are as defined above,
(i) —COR⁹, wherein R⁹ us as defined above,
(j) —CO₂R⁹, wherein R⁹ us as defined above;
(4) C₂₋₆ alkynyl;
(5) phenyl, unsubstituted or substituted with one or more of the substituents selected from:
(a) hydroxy,
(b) C₁₋₆ alkoxy,
(c) C₁₋₆ alkyl,
(d) C₂₋₅ alkenyl,
(e) halo,
(f) —CN,
(g) —NO₂,
(h) —CF₃,
(i) —(CH₂)ₘ—NR⁹R¹⁰, wherein m is 0, 1, or 2, R⁹ and R¹⁰ are as defined above,
(j) —NR⁹COR¹⁰, wherein R⁹ and R¹⁰ are as defined above,
(k) —NR⁹CO₂R¹⁰, wherein R⁹ and R¹⁰ are as defined above,
(1) —CONR⁹R¹⁰, wherein R⁹ and R¹⁰ are as defined above,
(m) —CO₂NR⁹R¹⁰, wherein R⁹ and R¹⁰ are as defined above,
(n) —COR⁹, wherein R⁹ is as defined above,
(o) CO₂R⁹, wherein R⁹ is as defined above;
(6) halo,
(7) —CN,
(8) —CF₃,
(9) —NO₂,
(10) —SR¹⁴, wherein R¹⁴ is hydrogen or C₁₋₅ alkyl,
(11) —SOR¹⁴, wherein R¹⁴ is as defined above,
(12) —SO₂R¹⁴, wherein R¹⁴ is as defined above,
(13) —NR⁹COR¹⁰, wherein R⁹ and R¹⁰ are as defined above,
(14) —CONR⁹COR¹⁰, wherein R⁹ and R¹⁰ are as defined above,
(15) —NR⁹R¹⁰, wherein R⁹ and R¹⁰ are as defined above,
(16) NR⁹CO₂R¹⁰, wherein R⁹ and R¹⁰ are as defined above,
(17) hydroxy,
(18) C₁₋₆ alkoxy,
(19) COR⁹, wherein R⁹ is as defined above,
(20) CO₂R⁹, wherein R⁹ is as defined above,
(21) 2-pyridyl,
(22) 3-pyridyl,
(23) 4-pyridyl,
(24) 5-tetrazolyl,
(25) 2-oxazolyl, and
(26) 2-thiazolyl;
R¹¹, R¹² and R¹³ are independently selected from the definitions of R⁶, R⁷ and R⁸;
A is selected from the group consisting of:
(1) C₁₋₆ alkyl, unsubstituted or substituted with one or more of the substituents selected from:
(a) hydroxy,
(b) oxo,
(c) C₁₋₆ alkoxy,
(d) phenyl-C₁₋₃ alkoxy,
(e) phenyl,
(f) —CN,
(g) halo, wherein halo is fluoro, chloro, bromo or iodo,
(h) —NR⁹R¹⁰, wherein R⁹ and R¹⁰ are as defined above,
(i) —NR⁹COR¹⁰, wherein R⁹ and R¹⁰ are as defined above,
(j) —NR⁹CO₂R¹⁰, wherein R⁹ and R¹⁰ are as defined above,
(k) —CONR⁹R¹⁰, wherein R⁹ and R¹⁰ are as defined above,
(l) COR⁹, wherein R⁹ is as defined above, and (m) —CO₂R⁹, wherein R⁹ is as defined above;
(2) C₂₋₆ alkenyl, unsubstituted or substituted with one or more of the substituent(s) selected from:
(a) hydroxy,
(b) oxo,
(c) C₁₋₆ alkoxy,
(d) phenyl-C₁₋₃ alkoxy,
(e) phenyl,
(f) —CN,
(g) halo,
(h) —CONR⁹R¹⁰, wherein R⁹ and R¹⁰ are as defined above,
(i) —COR⁹, wherein R⁹ is as defined above, and
(j) CO₂R⁹, wherein R⁹ is as defined above; and
(3) C₂₋₆ alkenyl;
B is a heterocycle, wherein the heterocycle is selected from the group consisting of: 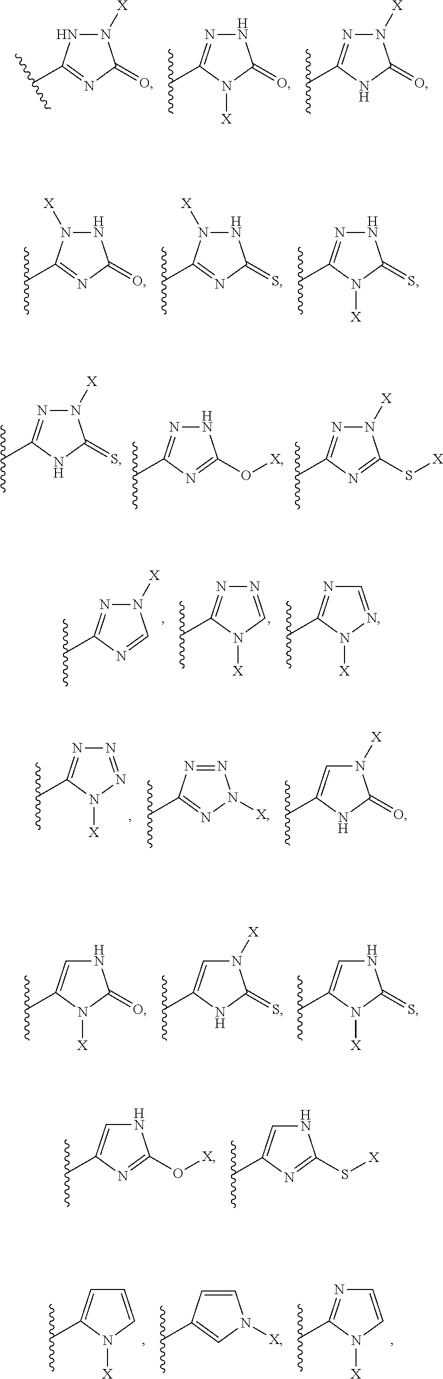 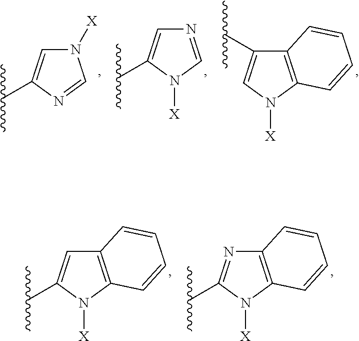
and wherein the heterocycle is substituted in addition to —X with one or more substituent(s) selected from:
(i) hydrogen;
(ii) C₁₋₆ alkyl, unsubstituted or substituted with halo, —CF₃, —OCH₃, or phenyl,
(iii) C₁₋₆ alkoxy,
(iv) oxo,
(v) hydroxy,
(vi) thioxo,
(vii) —SR⁹, wherein R⁹ is as defined above,
(viii) halo,
(ix) cyano,
(x) phenyl,
(xi) trifluoromethyl,
(xii) —(CH₂)ₘ—NR⁹R¹⁰, wherein m is 0, 1 or 2, and R⁹ and R¹⁰ are as defined above,
(xiii) —NR⁹COR¹⁰, wherein R⁹ and R¹⁰ are as defined above,
(xiv) —CONR⁹R¹⁰, wherein R⁹ and R¹⁰ are as defined above,
(xv) —CO₂R⁹, wherein R⁹ is as defined above, and
(xvi) —(CH₂)ₘ—OR⁹, wherein m and R⁹ are as defined above;
X is selected from:
(a) hydrogen,
(b) —PO(OH)₂
(c) —PO(OH)O⁻.M⁺, wherein M⁺ is a pharmaceutically acceptable monovalent counterion,
(d) —PO(O⁻)₂.2M⁺,
(e) —PO(O⁻)₂.D²⁺, wherein D²⁺ is a pharmaceutically acceptable divalent counterion,
(f) —CH(R⁴)—PO(OH)O⁻.M⁺, wherein R⁴ is hydrogen or C₁₋₃ alkyl,
(g) —CH(R⁴)—PO(O⁻)₂.2M⁺, and
(h) —CH(R⁴)—PO(O⁻)₂.D²⁺;
Y is selected from the group consisting of:
(1) a single bond,
(2) —O—,
(3) —S—,
(4) —CO—,
(5) —CH₂—,
(6) —CHR¹⁵—, and
(7) —CR¹⁵R¹⁶—, wherein R¹⁵ and R¹⁶ are independently selected from the group consisting of:
(a) C₁₋₆ alkyl, unsubstituted or substituted with one or more of the substituents selected from:
(i) hydroxy,
(ii) oxo,
(iii) C₁₋₆ alkoxy,
(iv) phenyl-C₁₋₃ alkoxy,
(v) phenyl,
(vi) —CN,
(vii) halo,
(viii) —NR⁹R¹⁰, wherein R⁹ and R¹⁰ are as defined above,
(ix) —NR⁹COR¹⁰, wherein R⁹ and R¹⁰ are as defined above,
(x) —NR⁹CO₂R¹⁰, wherein R⁹ and R¹⁰ are as defined above,
(xi) —CONR⁹R¹⁰, wherein R⁹ and R¹⁰ are as defined above,
(xii) —COR⁹, wherein R⁹ is as defined above, and
(xiii) —CO₂R⁹, wherein R⁹ is as defined above;
(b) phenyl, unsubstituted or substituted with one or more of the substituent(s) selected from:
(i) hydroxy,
(ii) C₁₋₆ alkoxy,
(iii) C₁₋₆ alkyl,
(iv) C₂₋₅ alkenyl,
(v) halo,
(vi) —CN,
(vii) —NO₂,
(viii) —CF₃,
(ix) —(CH₂)ₘ—NR⁹R¹⁰, wherein m, R⁹ and R¹⁰ are as defined above,
(x) —NR⁹COR¹⁰, wherein R⁹ and R¹⁰ are as defined above,
(xi) —NR⁹CO₂R¹⁰, wherein R⁹ and R¹⁰ are as defined above,
(xii) —CONR⁹R¹⁰, wherein R⁹ and R¹⁰ are as defined above,
(xiii) —CO₂NR⁹R¹⁰, wherein R⁹ and R¹⁰ are as defined above,
(xiv) —COR⁹, wherein R⁹ is as defined above, and
(xv) —CO₂R⁹, wherein R⁹ is as defined above;
Z is selected from:
(1) hydrogen,
(2) C₁₋₆ alkyl, and
(3) hydroxy, with the proviso that if Y is —O—, Z is other than hydroxy, or if Y is —CHR¹⁵—, then Z and R¹⁵ are optionally joined together to form a double bond.

36. A method according to claim 35, wherein said NK-1 receptor antagonist is selected from the group consisting of:
(1) 2-(R)-(1-(R)-(3,5-bis(trifluoromethyl)phenyl)ethoxy)-3-(S)-(4-fluoro)-phenyl-4-(3-(4-monophosphoryl-5-oxo-1H-1,2,4-triazolo)methyl)morpholine;
(2) 2-(R)-(1-(R)-(3,5-bis(trifluoromethyl)phenyl)ethoxy)-3-(S)-(4-fluoro)-phenyl-4-(3-(1-monophosphoryl-5-oxo-4H-1,2,4-triazolo)methyl)morpholine;
(3) 2-(R)-(1-(R)-(3,5-bis(trifluoromethyl)phenyl)ethoxy)-3-(S)-(4-fluoro)-phenyl-4-(3-(2-monophosphoryl-5-oxo-1H-1,2,4-triazolo)methyl)morpholine;
(4) 2-(R)-(1-(R)-(3,5-bis(trifluoromethyl)phenyl)ethoxy)-3-(S)-(4-fluoro)-phenyl-4-(3-(5-oxyphosphoryl-1H-1,2,4-triazolo)-methyl)morpholine;
(5) 2-(S)-(1-(R)-(3,5-bis(trifluoromethyl)phenyl)ethoxy)-3-(S)-(4-fluoro)-phenyl-4-(3-(1-monophosphoryl-5-oxo-4H-1,2,4-triazolo)methyl)morpholine;
(6) 2-(S)-(1-(R)-(3,5-bis(trifluoromethyl)phenyl)ethoxy)-3-(S)-(4-fluoro)-phenyl-4-(3-(1-monophosphoryl-5-oxo-4H-1,2,4-triazolo)methyl)morpholine tautomers thereof, pharmaceutically acceptable salts thereof and pharmaceutically acceptable salts of said tautomers.

37. A method according to claim 35, wherein said NK-1 receptor antagonist is a compound of Formula (I) including tautomers, pharmaceutically acceptable salts thereof or a pharmaceutically acceptable salt of said tautomers, wherein said pharmaceutically acceptable salt is the bis(N-methyl-D-glucamine) salt.

38. A method according to claim 35, wherein said NK-1 receptor antagonist is selected from the group consisting of: 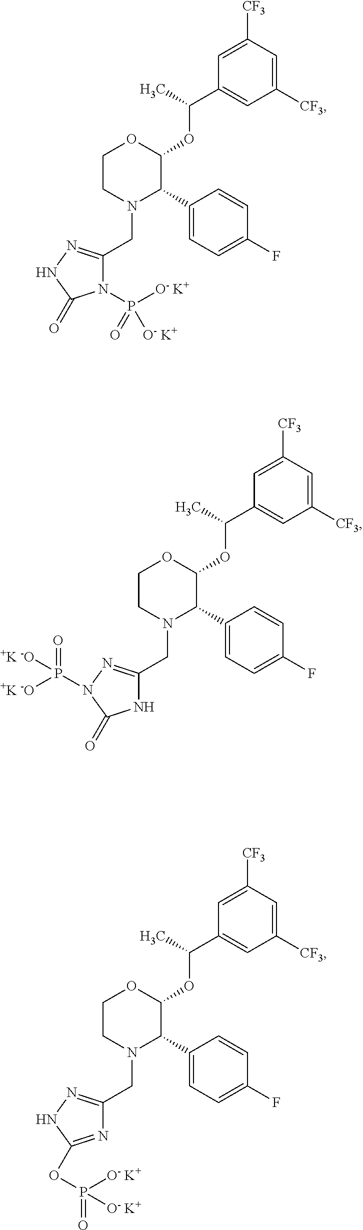 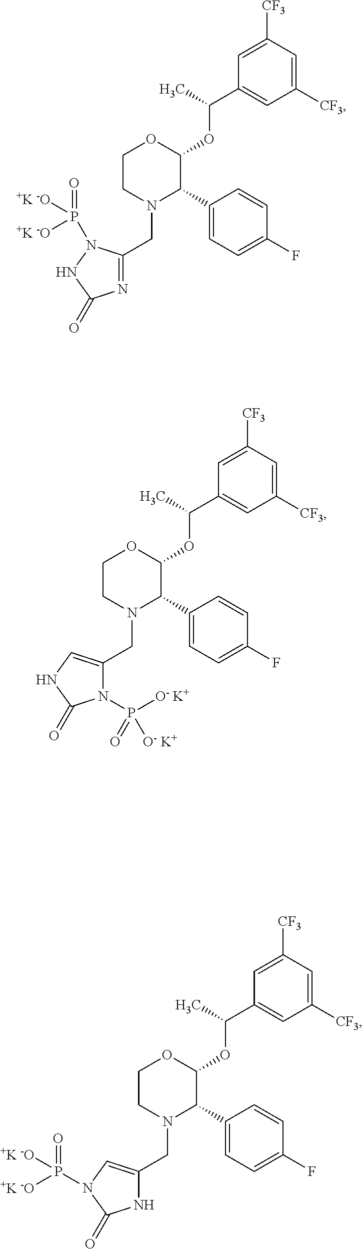 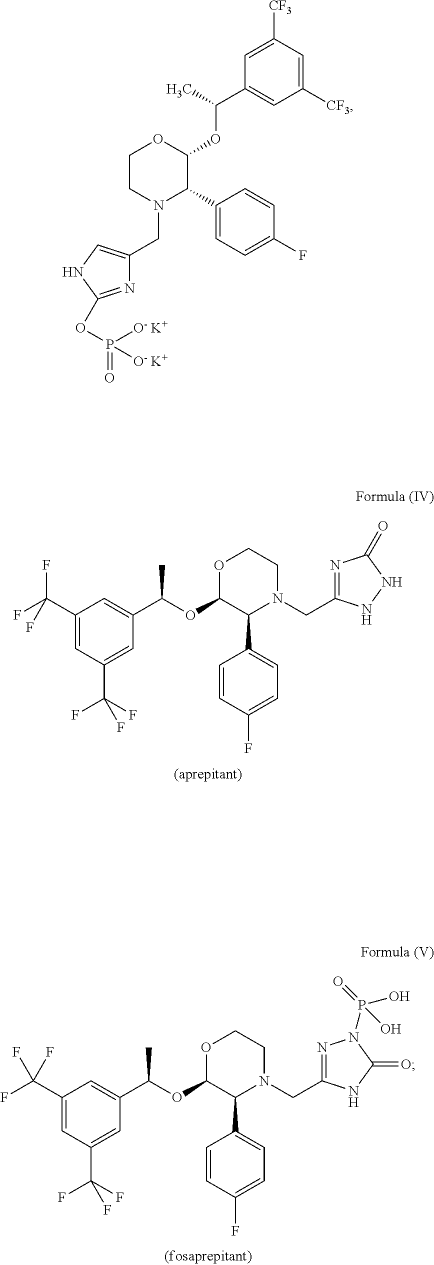
including tautomers thereof, pharmaceutically acceptable salts thereof or a pharmaceutically acceptable salt of said tautomers,
wherein K⁺ is a pharmaceutically acceptable monovalent counterion.

39. A method according to claim 38, wherein K⁺ is N-methyl D-glucamine.

40. A method according to claim 35, wherein said NK-1 receptor antagonist is selected from a group consisting of: 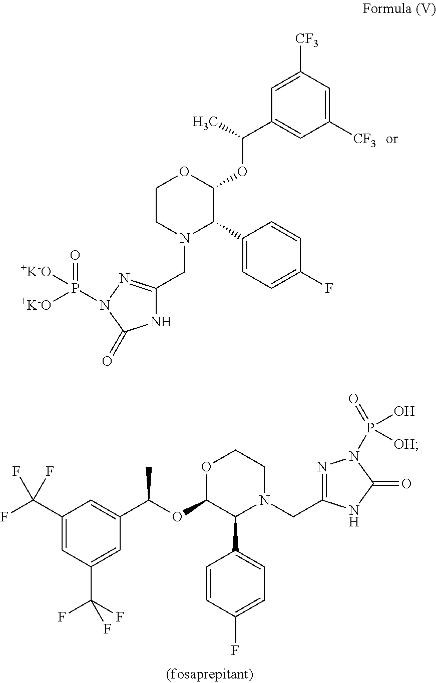
including tautomers thereof, pharmaceutically acceptable salts thereof or a pharmaceutically acceptable salt of said tautomers,
wherein K⁺ is a pharmaceutically acceptable monovalent counterion.

41. A method according to claim 40, wherein K⁺ is N methyl-D-glucamine.

42. A method according to claim 35, wherein said NK-1 receptor antagonist is aprepitant including tautomers thereof, pharmaceutically acceptable salts thereof or a pharmaceutically acceptable salt of said tautomers.

43. A method according to claim 35, wherein said NK-1 receptor antagonist is administered orally.

44. A method according to claim 35, wherein said NK-1 receptor antagonist is aprepitant including tautomers, pharmaceutically acceptable salts thereof or a pharmaceutically acceptable salt of said tautomers and wherein said NK-1 receptor antagonist is administered orally.

45. A method according to claim 35, wherein said NK-1 receptor antagonist is administered intravenously.

46. A method according to claim 45, wherein said NK-1 receptor antagonist is fosaprepitant including tautomers, pharmaceutically acceptable salts thereof or a pharmaceutically acceptable salt of said tautomers and wherein said NK-1 receptor antagonist is administered intravenously.

47. A method according to claim 35 wherein the dosage level of said NK-1 receptor antagonist is in an effective amount of 2 to 200 mg/kg per day and/or wherein said NK-1 receptor antagonist is administered to a patient over a period of at least 2 days.

48. A method according to claim 35, wherein said NK-1 receptor antagonist is administered as a monotherapeutic, without another anticancer or cancer preventing drug.

49. A method according to claim 48, wherein said NK-1 receptor antagonist is not combined with a chemotherapeutic agent.

50. A NK-1 receptor antagonist according claim 35, wherein said NK-1 receptor antagonist is administered with a chemotherapeutic agent.

51. A method according to claim 35, wherein said NK-1 receptor antagonist is administered to a female subject having triple negative breast cancer.

52. A method according to claim 51, wherein said NK-1 receptor antagonist is aprepitant or fosaprepitant including tautomers thereof, pharmaceutically acceptable salts thereof or a pharmaceutically acceptable salt of said tautomers.

53. A method according to claim 51, wherein said NK-1 receptor antagonist is administered either as a a monotherapeutic, without another anticancer or cancer preventing drug or alternatively in combination with another anticancer or cancer preventing drug.

54. A method according to claim 52, wherein the dosage level of said NK-1 receptor antagonist is in an effective amount of 2 to 200 mg/kg per day and/or administered to a patient over a period of at least 2 days.

55. A pharmaceutical composition for oral administration comprising an amount of a NK-1 receptor antagonist according to claim 35 effective for preventing cancer in the range of 100 to 10 g.

56. A pharmaceutical composition for oral administration comprising an amount of a NK-1 receptor antagonist according to claim 35 effective for preventing cancer in the range of 150 mg to 10 g.

57. A pharmaceutical composition comprising an amount of a NK-1 receptor antagonist according to claim 35 effective for use in a method of preventing cancer.

58. A pharmaceutical composition according to claim 55 effective for use in a method of preventing cancer wherein said pharmaceutical composition is to be used as monotherapeutic, without another anticancer or cancer preventing drug.

59. A pharmaceutical composition according to claim 55 effective for use in a method of preventing cancer wherein said pharmaceutical composition is to be used in combination with another anticancer or cancer preventing drug.

60. A pharmaceutical composition according to claim 59 effective for use in a method of preventing cancer wherein said another anticancer or cancer preventing drug is a chemotherapeutic agent.

61. A pharmaceutical composition according to claim 55 effective for use in a method of preventing cancer, wherein said pharmaceutical composition is administered to a patient over a period of at least 2 days.

62. A pharmaceutical composition according to claim 55 effective for use in a method of preventing cancer wherein said pharmaceutical composition is administered to a subject having not cancer but having an enhanced risk of getting cancer, wherein said subject is selected from the group consisting of:
a subject having had cancer but has been cured from cancer
a female subject having had breast cancer but has been cured from breast cancer
a female subject receiving Hormone Replacement Therapy
a subject having a genetic predisposition to get cancer, e.g. a female subject having a BRCA mutation
a subject with a family history of getting cancer
a subject with history or prevalence of a disease associated with an elevated risk to develop cancer, such as for instance, such as for instance diabetes
a subject, who uses or used to smoke
a subject with a score-based elevated risk to get cancer
a female subject having an elevated pro-Neurotensin level in a sample of fastening blood and
a (female) subject having an reduced level of PENK or fragments thereof including MRPENK in a blood sample.

63. A pharmaceutical composition according to claim 55 effective for use in a method of preventing cancer wherein said pharmaceutical composition is administered to a subject having not cancer but having an enhanced risk of getting cancer, wherein said subject is a female subject and wherein a female subject having an enhanced risk of getting cancer may be identified as follows:
determining the level of pro-Neurotensin or fragments thereof of at least 5 amino acids in a bodily fluid obtained from said fasting female subject; and
correlating said level of pro-Neurotensin or fragments thereof with a risk for getting cancer, wherein an elevated level is predictive for an enhanced risk of getting cancer.

64. A pharmaceutical composition according to claim 55 effective for use in a method of preventing cancer wherein said pharmaceutical composition is administered to a subject having not cancer but having an enhanced risk of getting cancer, wherein said subject having an enhanced risk of getting cancer may be identified as follows:
determining the level of Pro-Enkephalin (PENK) or fragments thereof including MRPENK Leu-Enkephalin and Met-Enkephalin of at least 5 amino acids in a bodily fluid obtained from said female subject; and
correlating said level of Pro-Enkephalin or fragments thereof with a risk for getting cancer, wherein a reduced level is predictive for an enhanced risk of getting cancer.

65. A pharmaceutical composition according to claim 55 effective for use in a method of preventing cancer wherein said pharmaceutical composition is administered to a subject having not cancer but having an enhanced risk of getting cancer, wherein said subject has had triple negative breast cancer.

66. A pharmaceutical composition according to claim 55 effective for use in a method of preventing cancer wherein said pharmaceutical composition is to be used in combination with one or more agents which are tamoxifen, raloxifene, anastrozole or exemestane.

67. A NK-1 receptor antagonist in a form effective for use as a cancer preventing agent, selected from the group consisting of:
(1) 2-(R)-(1-(R)-(3,5-bis(trifluoromethyl)phenyl)ethoxy)-3-(S)-(4-fluoro)-phenyl-4-(3-(4-monophosphoryl-5-oxo-1H-1,2,4-triazolo)methyl)morpholine;
(2) 2-(R)-(1-(R)-(3,5-bis(trifluoromethyl)phenyl)ethoxy)-3-(S)-(4-fluoro)-phenyl-4-(3-(1-monophosphoryl-5-oxo-4H-1,2,4-triazolo)methyl)morpholine;
(3) 2-(R)-(1-(R)-(3,5-bis(trifluoromethyl)phenyl)ethoxy)-3-(S)-(4-fluoro)-phenyl-4-(3-(2-monophosphoryl-5-oxo-1H-1,2,4-triazolo)methyl)morpholine;
(4) 2-(R)-(1-(R)-(3,5-bis(trifluoromethyl)phenyl)ethoxy)-3-(S)-(4-fluoro)-phenyl-4-(3-(5-oxyphosphoryl-1H-1,2,4-triazolo)-methyl)morpholine;
(5) 2-(S)-(1-(R)-(3,5-bis(trifluoromethyl)phenyl)ethoxy)-3-(S)-(4-fluoro)-phenyl-4-(3-(1-monophosphoryl-5-oxo-4H-1,2,4-triazolo)methyl)morpholine;
(6) 2-(S)-(1-(R)-(3,5-bis(trifluoromethyl)phenyl)ethoxy)-3-(S)-(4-fluoro)-phenyl-4-(3-(1-monophosphoryl-5-oxo-4H-1,2,4-triazolo)methyl)morpholine, tautomers thereof, pharmaceutically acceptable salts thereof and pharmaceutically acceptable salt of said tautomers.

68. A NK-1 receptor antagonist in a form effective for use as a cancer preventing agent, selected from the group consisting of: 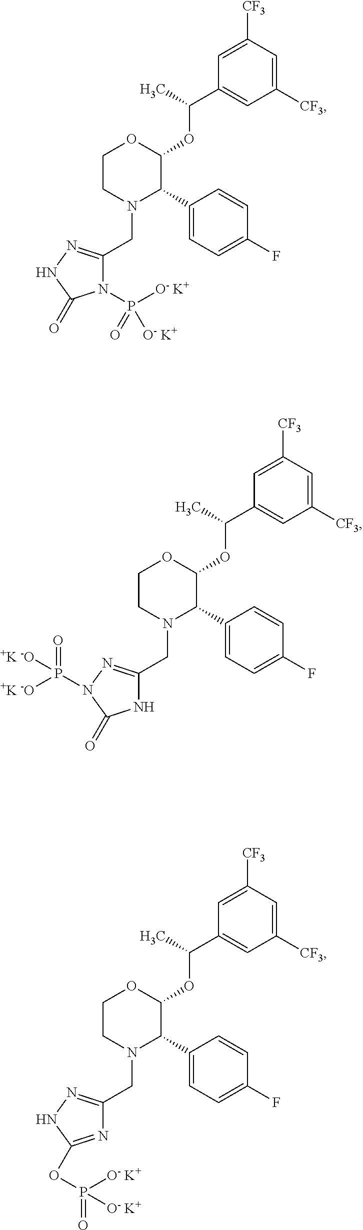 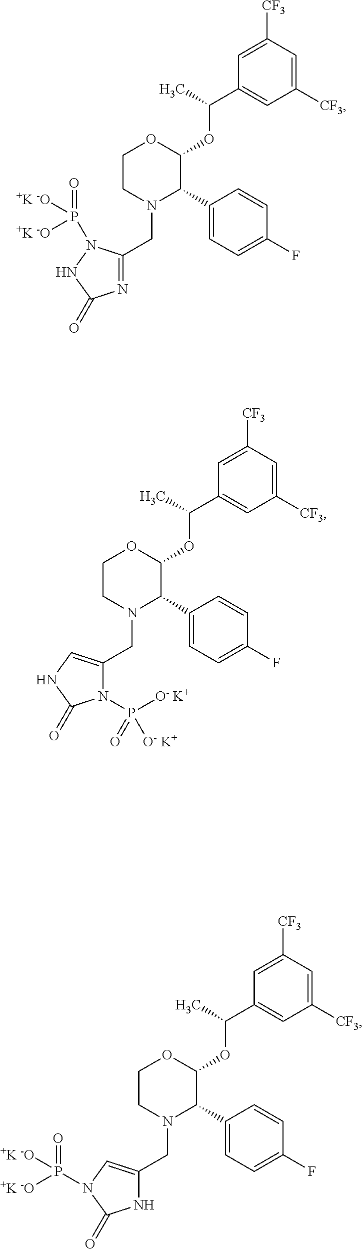 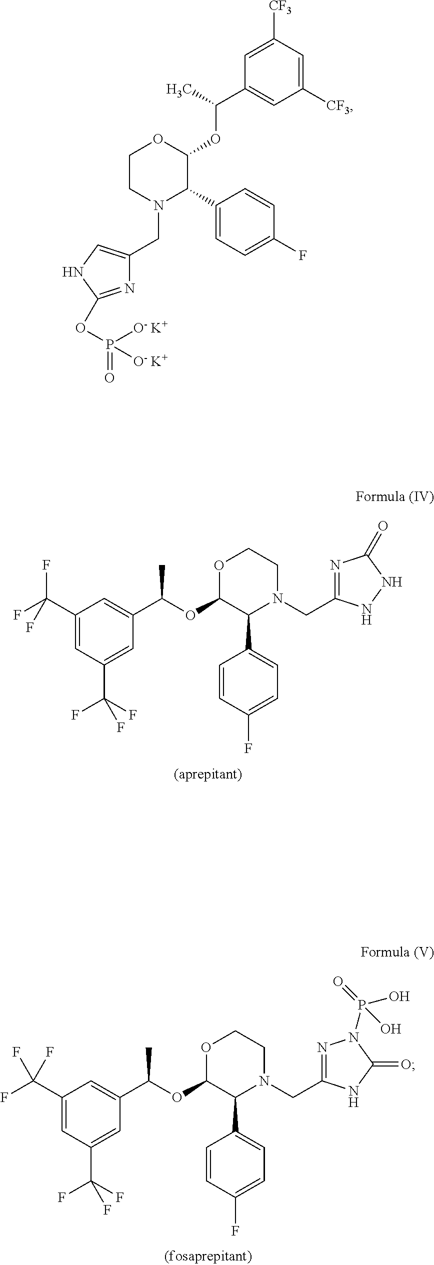
including tautomers thereof, pharmaceutically acceptable salts thereof or a pharmaceutically acceptable salt of said tautomers,
wherein K⁺ is a pharmaceutically acceptable monovalent counterion.
